SEQUENCE LISTING (1 sequence)

SEQ ID NO: 1 - DNA, 16 bp, Adeno-associated virus-2
gcgcgctcgctcgctc

(19) United States
(12) Patent Application Publication
(10) Pub. No.: US 2022/0280427 A1
(43) Pub. Date: Sep. 8, 2022

(54) LIPID NANOPARTICLE COMPOSITIONS COMPRISING CLOSED-ENDED DNA AND CLEAVABLE LIPIDS AND METHODS OF USE THEREOF

(71) Applicant: Generation Bio Co., Cambridge, MA (US)

(72) Inventors: Jie Su, Cambridge, MA (US); Prudence Yui Tung Li, Cambridge, MA (US); Debra Klatte, Cambridge, MA (US); Leah Yu Liu, Cambridge, MA (US); Matthew James Chiocco, Cambridge, MA (US); Matthew G. Stanton, Cambridge, MA (US); Jeff Moffit, Cambridge, MA (US); Jon Edward Chatterton, Cambridge, MA (US)

(21) Appl. No.: 17/632,262

(22) PCT Filed: Sep. 3, 2020
(86) PCT No.: PCT/US2020/049266

§ 371 Date: Feb. 2, 2022

Related U.S. Application Data
(60) Provisional application No. 62/940,104, filed on Nov. 25, 2019.
(60) Provisional application No. 62/910,720, filed on Oct. 4, 2019.
(60) Provisional application No. 62/896,980, filed on Sep. 6, 2019.

(51) Int. Cl. A61K 9/127; A61K 48/00; A61K 31/573

(52) U.S. Cl. CPC A61K 9/1272; A61K 48/0033; A61K 48/0058; A61K 31/573

(57) ABSTRACT

Provided herein are lipid formulations comprising a lipid and a capsid free, non-viral vector (e.g. ceDNA). Lipid particles (e.g., lipid nanoparticles) of the invention include a lipid formulation that can be used to deliver a capsid-free, non-viral DNA vector to a target site of interest (e.g., cell, tissue, organ, and the like).

### RELATED APPLICATIONS

This application claims priority to U.S. Provisional Application No. 62/896,980, filed on Sep. 6, 2019, U.S. Provisional Application No. 62/910,720, filed on Oct. 4, 2019 and U.S. Provisional Application No. 62/940,104, filed on Nov. 25, 2019, the contents of each of which are hereby incorporated by reference in their entireties.

### SEQUENCE LISTING

The instant application contains a Sequence Listing which has been submitted electronically in ASCII format. Said ASCII copy, created on Sep. 3, 2020, is named 131698-07520_SL.txt and is 556 bytes in size.

### BACKGROUND

Gene therapy aims to improve clinical outcomes for patients suffering from either genetic disorders or acquired diseases caused by an aberrant gene expression profile. Various types of gene therapy that deliver therapeutic nucleic acids into a patient's cells as a drug to treat disease have been developed to date. Generally, gene therapy involves treatment or prevention of medical conditions resulting from defective genes or abnormal regulation or expression, e.g., under- or over-expression, that can result in a disorder, disease, or malignancy. For example, a disease or disorder caused by a defective gene might be treated by delivery of a corrective genetic material to a subject to supplement the defective gene and bolster the wild-type copy of the gene by providing a wild type copy of the gene. In some cases, treatment is achieved by delivery of therapeutic nucleic acid molecules that modulate expression of the defective gene at the transcriptional of translational level, either providing an antisense nucleic acid that binds the target DNA or mRNA that brings down expression levels of the defective gene, or by transferring wild-type mRNA to increase correct copies of the gene.

In particular, human monogenic disorders have been treated by the delivery and expression of a normal gene to the target cells. Delivery and expression of a corrective gene in the patient's target cells can be carried out via numerous methods, including the use of engineered viral gene delivery vectors, and potentially plasmids, minigenes, oligonucleotides, minicircles, or variety of closed-ended DNAs. Among the many virus-derived vectors available (e.g., recombinant retrovirus, recombinant lentivirus, recombinant adenovirus, and the like), recombinant adeno-associated virus (rAAV) is gaining acceptance as a versatile, as well as relatively reliable, vector in gene therapy. However, viral vectors, such as adeno-associated vectors, can be highly immunogenic and elicit humoral and cell-mediated immunity that can compromise efficacy, particularly with respect to re-administration.

Molecular sequences and structural features encoded in the AAV viral genome/vector have evolved to promote episomal stability, viral gene expression and interact with the host's immune system. AAV vectors contain hairpin DNA structures conserved throughout the AAV family, which play critical roles in essential functions of AAV, the ability to tap into the host's genome and replicate themselves, while escaping the surveillance system of the host.

However, some of these gene therapy modalities suffer greatly from the immune related adverse events, which are closely related to host's own defensive mechanism against the therapeutic nucleic acid. For example, the immune system has two general mechanisms for combating infectious diseases that have been implicated in causing adverse events in the recipients of therapy. The first is known as the “innate” immune response that is typically triggered within minutes of infection and serves to limit the pathogen's spread in vivo. The host recognizes conserved determinants expressed by a diverse range of infectious microorganisms, but absent from the host, and these determinants stimulate elements of the host's innate immune system to produce immunomodulatory cytokines and polyreactive IgM antibodies. The second and subsequent mechanism is known as an “adaptive” or antigen specific immune response, which typically generated against determinants expressed uniquely by the pathogen. The innate and adaptive immune responses are mainly activated and modulated by a set of type I interferons (IFNs) through a set of signaling pathways that are activated by specific type of nucleic acids.

Non-viral gene delivery circumvents certain disadvantages associated with viral transduction, particularly those due to the humoral and cellular immune responses to the viral structural proteins that form the vector particle, and any de novo virus gene expression. Non-viral gene transfer typically uses bacterial plasmids to introduce foreign DNA into recipient cells. In addition to the transgene of interest, such DNAs routinely contain extraneous sequence elements needed for selection and amplification of the plasmid DNA (pDNA) in bacteria, such as antibiotic resistance genes and a prokaryotic origin of replication. For example, plasmids produced in E. coli contain elements needed for propagation in prokaryotes, such as a prokaryotic origin of DNA replication and a selectable marker, as well as uniquely prokaryotic modifications to DNA, that are unnecessary, and that can be deleterious, for transgene expression in mammalian cells.

Although conceptually elegant, the prospect of using nucleic-acid molecules for gene therapy for treating human diseases remains uncertain. The main cause of this uncertainty is the apparent adverse events relating to host's innate immune response to nucleic acid therapeutics and, thus, the way in which these materials modulate expression of their intended targets in the context of the immune response. The current state of the art surrounding the creation, function, behavior and optimization of nucleic acid molecules that may be adopted for clinical applications has a particular focus on: (1) antisense oligonucleotides and duplex RNAs that directly regulate translation and gene expression; (2) transcriptional gene silencing RNAs that result in long-term epigenetic modifications; (3) antisense oligonucleotides that interact with and alter gene splicing patterns; (4) creation of synthetic or viral vectors that mimic physiological functionalities of naturally occurring AAV or lentiviral genome; and (5) the in vivo delivery of therapeutic oligonucleotides. However, despite the advances made in the development of nucleic acid therapeutics that are evident in recent clinical achievements, the field of gene therapy is still severely limited by unwanted adverse events in recipients triggered by the therapeutic nucleic acids, themselves.

Accordingly, there is a strong need in the field for a new technology that effectively reduces, ameliorates, mitigates, prevents or maintains the immune response systems that are triggered by nucleic acid therapeutics.

### SUMMARY

Provided herein are pharmaceutical compositions comprising a cationic lipid, e.g., a ionizable cationic lipid, e.g., an SS-cleavable lipid, and a capsid free, non-viral vector (e.g., ceDNA) that can be used to deliver the capsid-free, non-viral DNA vector to a target site of interest (e.g., cell, tissue, organ, and the like), as well as methods of use and manufacture thereof. Surprisingly, and as demonstrated herein, lipid nanoparticles (LNPs) comprising a cleavable lipid provide more efficient delivery of therapeutic nucleic acids, e.g., ceDNA, to target cells (including, e.g., hepatic cells). In particular, a ceDNA particle comprising ceDNA and a cleavable lipid resulted in fewer ceDNA copies in liver tissue samples with equivalent protein expression as compared to other lipids, e.g., MC3. Although the mechanism has not yet been determined, and without being bound by theory, it is thought that the ceDNA containing lipid particles (e.g., lipid nanoparticles) comprising a SS-cleavable lipid provide improved delivery to hepatocytes versus non-parenchymal cells and more efficient trafficking to the nucleus. Another advantage of the ceDNA lipid particles (e.g., lipid nanoparticles) comprising a cleavable lipid described herein is better tolerability compared to other lipids (e.g., other ionizable cationic lipids, e.g., MC3), shown by reduced body weight loss and decreased cytokine release. The beneficial effect on tolerability can be further enhanced by adding an immunosuppressant conjugate (e.g., dexamethasone palmitate) or a tissue specific ligand (e.g., N-Acetylgalatosamine (GalNAc)) to the LNPs of the present disclosure. Surprisingly, it was discovered that ceDNA formulated in SS-cleavable lipids described herein successfully avoids phagocytosis by immune cells (see, for example, FIGS. 13-15) as compared to ceDNA formulated in other lipids, e.g., MC3 and may lead to higher expression per copy number in a target cell or organ (e.g., liver). Indeed, a synergistic effect can occur between the ceDNA formulated in SS-cleavable lipid (e.g., ss-OP4) and GalNAc such that the ceDNA-LNPs comprising SS-cleavable lipid and GalNAc may exhibit approximately up to 4,000-fold greater hepatocyte targeting as compared to that seen with ceDNA formulated in the SS-cleavable lipid only (ss-OP4) (FIGS. 18A and 18B), while ceDNA formulated in typical cationic lipids with GalNAc demonstrated merely approximately 10-fold greater hepatocyte targeting. Moreover, it was discovered that ceDNA formulated in SS-cleavable lipid (ss-OP4) with GalNAc showed an improved safety profile in term of complement and cytokine responses.

In one aspect, disclosed herein is a pharmaceutical composition comprising a lipid nanoparticle (LNP), wherein the LNP comprises a SS-cleavable lipid and a therapeutic nucleic acid (TNA). In another aspect, disclosed herein is a pharmaceutical composition comprising a lipid nanoparticle (LNP), wherein the LNP comprises a SS-cleavable lipid and an mRNA. In one aspect, disclosed herein is a pharmaceutical composition comprising a lipid nanoparticle (LNP), wherein the LNP comprises a SS-cleavable lipid and a closed-ended DNA (ceDNA). According to some embodiments, the SS-cleavable lipid comprises a disulfide bond and a tertiary amine. According to some embodiments of any of the aspects or embodiments herein, the SS-cleavable lipid comprises an ss-OP lipid of Formula I:

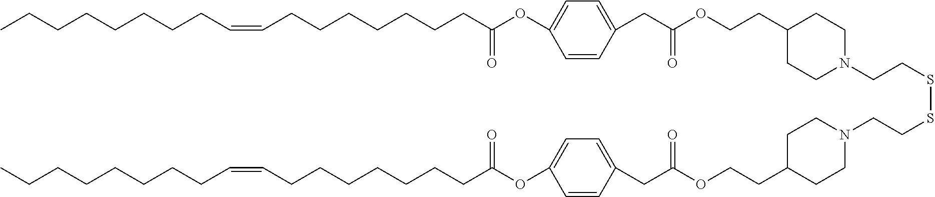 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

According to some embodiments of any of the aspects or embodiments herein, the LNP further comprises a sterol. According to some embodiments, the sterol is a cholesterol. According to some embodiments of any of the aspects or embodiments herein, the LNP further comprises a polyethylene glycol (PEG). According to some embodiments, the PEG is 1-(monomethoxy-polyethyleneglycol)-2,3-dimyristoylglycerol (PEG-DMG). According to some embodiments of any of the aspects or embodiments herein, the LNP further comprises a non-cationic lipid. According to some embodiments, the non-cationic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. According to some embodiments, the non-cationic lipid is selected from the group consisting of dioleoylphosphatidylcholine (DOPC), distearoylphosphatidylcholine (DSPC), and dioleoyl-phosphatidylethanolamine (DOPE).

According to some embodiments, the PEG or PEG-lipid conjugate is present at about 1.5% to about 3%, for example about 1.5% to about 2.75%, about 1.5% to about 2.5%, about 1.5% to about 2.25%, about 1.5% to about 2%, about 1.5% to about 1.75%, about 2% to about 3%, about 2% to about 2.75%, about 2% to about 2.5%, about 2% to about 2.25%. According to some embodiments, the PEG or PEG-lipid conjugate is present at about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, or about 3%. According to some embodiments, the cholesterol is present at a molar percentage of about 20% to about 40%, for example about 20% to about 35%, about 20% to about 30%, about 20% to about 25%, about 25% to about 35%, about 25% to about 30%, or about 30% to about 35%, and the SS-cleavable lipid is present at a molar percentage of about 80% to about 60%, for example about 80% to about 65%, about 80% to about 70%, about 80% to about 75%, about 75% to about 60%, about 75% to about 65%, about 75% to about 70%, about 70% to about 60%, or about 70% to about 60%. According to some embodiments, the cholesterol is present at a molar percentage of about 20% to about 40%, for example about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, and wherein the SS-cleavable lipid is present at a molar percentage of about 80% to about 60%, for example about 80%, about 79%, about 78%, about 77%, about 76%, about 75%, about 74%, about 73%, about 72%, about 71%, about 70%, about 69%, about 68%, about 67%, about 66%, about 65%, about 64%<about 63%, about 62%, about 61%, or about 60%. According to some embodiments, the cholesterol is present at a molar percentage of about 40%, and wherein the SS-cleavable lipid is present at a molar percentage of about 50%. According to some embodiments of any of the aspects or embodiments herein, the composition further comprises a cholesterol, a PEG or PEG-lipid conjugate, and a non-cationic lipid. According to some embodiments, the PEG or PEG-lipid conjugate is present at about 1.5% to about 3%, for example about 1.5% to about 2.75%, about 1.5% to about 2.5%, about 1.5% to about 2.25%, about 1.5% to about 2%, about 2% to about 3%, about 2% to about 2.75%, about 2% to about 2.5%, about 2% to about 2.25%, about 2.25% to about 3%, about 2.25% to about 2.75%, or about 2.25% to about 2.5%. According to some embodiments, the PEG or PEG-lipid conjugate is present at about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, or about 3%. According to some embodiments, the cholesterol is present at a molar percentage of about 30% to about 50%, for example about 30% to about 45%, about 30% to about 40%, about 30% to about 35%, about 35% to about 50%, about 35% to about 45%, about 35% to about 40%, about 40% to about 50%, or about 45% to about 50%. According to some embodiments, the cholesterol is present at a molar percentage of about 30%, about 31%, about 32%, about 33%, about 34%, about 35%, about 36%, about 37%, about 38%, about 39%, about 40%, about 41%, about 42%, about 43%, about 44%, about 45%, about 46%, about 4′7%, about 48%, about 49%, or about 50%. According to some embodiments, the SS-cleavable lipid is present at a molar percentage of about 42.5% to about 62.5%. According to some embodiments, the SS-cleavable lipid is present at a molar percentage of about 42.5%, about 43%, about 43.5%, about 44%, about 44.5%, about 45%, about 45.5%, about 46%, about 46.5%, about 4′7%, about 4′7.5%, about 48%, about 48.5%, about 49%, about 49.5%, about 50%, about 50.5%, about 51%, 51.5%, about 52%, about 52.5%, about 53%, about 53.5%, about 54%, about 54.5%, about 55%, about 55.5%, about 56%, about 56.5%, about 57%, 57.5%, about 58%, about 58.5%, about 59%, about 59.5%, about 60%, about 60.5%, about 61%, about 61.5%, about 62%, or about 62.5%. According to some embodiments of any of the aspects or embodiments herein, the non-cationic lipid is present at a molar percentage of about 2.5% to about 12.5%. According to some embodiments of any of the aspects or embodiments herein, the cholesterol is present at a molar percentage of about 40%, the SS-cleavable lipid is present at a molar percentage of about 52.5%, the non-cationic lipid is present at a molar percentage of about 7.5%, and wherein the PEG is present at about 3%. According to some embodiments of any of the aspects or embodiments herein, the composition further comprises dexamethasone palmitate. According to some embodiments of any of the aspects or embodiments herein, the LNP is in size ranging from about 50 nm to about 110 nm in diameter, for example about 50 nm to about 100 nm, about 50 nm to about 95 nm, about 50 nm to about 90 nm, about 50 nm to about 85 nm, about 50 nm to about 80 nm, about 50 nm to about 75 nm, about 50 nm to about 70 nm, about 50 nm to about 65 nm, about 50 nm to about 60 nm, about 50 nm to about 55 nm, about 60 nm to about 110 nm, about 60 nm to about 100 nm, about 60 nm to about 95 nm, about 60 nm to about 90 nm, about 60 nm to about 85 nm, about 60 nm to about 80 nm, about 60 nm to about 75 nm, about 60 nm to about 70 nm, about 60 nm to about 65 nm, about 70 nm to about 110 nm, about 70 nm to about 100 nm, about 70 nm to about 95 nm, about 70 nm to about 90 nm, about 70 nm to about 85 nm, about 70 nm to about 80 nm, about 70 nm to about 75 nm, about 80 nm to about 110 nm, about 80 nm to about 100 nm, about 80 nm to about 95 nm, about 80 nm to about 90 nm, about 80 nm to about 85 nm, about 90 nm to about 110 nm, or about 90 nm to about 100 nm. According to some embodiments of any of the aspects or embodiments herein, the LNP is less than about 100 nm in size, for example less than about 105 nm, less than about 100 nm, less than about 95 nm, less than about 90 nm, less than about 85 nm, less than about 80 nm, less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, less than about 20 nm, less than about 15 nm, or less than about 10 nm in size. According to some embodiments, the LNP is less than about 70 nm in size, for example less than about 65 nm, less than about 60 nm, less than about 55 nm, less than about 50 nm, less than about 45 nm, less than about 40 nm, less than about 35 nm, less than about 30 nm, less than about 25 nm, less than about 20 nm, less than about 15 nm, or less than about 10 nm in size. According to some embodiments, the LNP is less than about 60 nm in size, for example less than about 55 nm, less than about 50 nm, less than about 45 nm, less than about 40 nm, less than about 35 nm, less than about 30 nm, less than about 25 nm, less than about 20 nm, less than about 15 nm, or less than about 10 nm in size. According to some embodiments of any of the aspects or embodiments herein, the composition has a total lipid to ceDNA ratio of about 15:1. According to some embodiments of any of the aspects or embodiments herein, the composition has a total lipid to ceDNA ratio of about 30:1. According to some embodiments of any of the aspects or embodiments herein, the composition has a total lipid to ceDNA ratio of about 40:1. According to some embodiments of any of the aspects or embodiments herein, the composition has a total lipid to ceDNA ratio of about 50:1. According to some embodiments of any of the aspects or embodiments herein, the composition further comprises N-Acetylgalactosamine (GalNAc). According to some embodiments, the GalNAc is present in the LNP at a molar percentage of 0.2% of the total lipid. According to some embodiments, the GalNAc is present in the LNP at a molar percentage of 0.3% of the total lipid. According to some embodiments, the GalNAc is present in the LNP at a molar percentage of 0.4% of the total lipid. According to some embodiments, the GalNAc is present in the LNP at a molar percentage of 0.5% of the total lipid. According to some embodiments, the GalNAc is present in the LNP at a molar percentage of 0.6% of the total lipid. According to some embodiments, the GalNAc is present in the LNP at a molar percentage of 0.7% of the total lipid. According to some embodiments, the GalNAc is present in the LNP at a molar percentage of 0.8% of the total lipid. According to some embodiments, the GalNAc is present in the LNP at a molar percentage of 0.9% of the total lipid. According to some embodiments, the GalNAc is present in the LNP at a molar percentage of 1.0% of the total lipid. According to some embodiments, the GalNAc is present in the LNP at a molar percentage of about 1.5% of the total lipid. According to some embodiments, the GalNAc is present in the LNP at a molar percentage of 2.0% of the total lipid. According to some embodiments of any of the aspects or embodiments herein, the composition further comprises about 10 mM to about 30 mM malic acid, for example about 10 mM to about 25 mM, about 10 mM to about 20 mM, about 10 mM to about 15 mM, about 15 mM to about 25 mM, about 15 mM to about 20 mM, about 20 mM to about 25 mM. According to some embodiments of any of the aspects or embodiments herein, the composition further comprises about 10 mM malic acid, about 11 mM malic acid, about 12 mM malic acid, about 13 mM malic acid, about 14 mM malic acid, about 15 mM malic acid, about 16 mM malic acid, about 17 mM malic acid, about 18 mM malic acid, about 19 mM malic acid, about 20 mM malic acid, about 21 mM malic acid, about 22 mM malic acid, about 23 mM malic acid, about 24 mM malic acid, about 25 mM malic acid, about 26 mM malic acid, about 27 mM malic acid, about 28 mM malic acid, about 29 mM malic acid, or about 30 mM malic acid. According to some embodiments, the composition comprises about 20 mM malic acid. According to some embodiments of any of the aspects or embodiments herein, the composition further comprises about 30 mM to about 50 mM NaCl, for example about 30 mM to about 45 mM NaCl, about 30 mM to about 40 mM NaCl, about 30 mM to about 35 mM NaCl, about 35 mM to about 45 mM NaCl, about 35 mM to about 40 mM NaCl, or about 40 mM to about 45 mM NaCl. According to some embodiments of any of the aspects or embodiments herein, the composition further comprises about 30 mM NaCl, about 35 mM NaCl, about 40 mM NaCl, or about 45 mM NaCl. According to some embodiments, the composition comprises about 40 mM NaCl. According to some embodiments, the composition further comprises about 20 mM to about 100 mM MgCl₂, for example about 20 mM to about 90 mM MgCl₂, about 20 mM to about 80 mM MgCl₂, about 20 mM to about 70 mM MgCl₂, about 20 mM to about 60 mM MgCl₂, about 20 mM to about 50 mM MgCl₂, about 20 mM to about 40 mM MgCl₂, about 20 mM to about 30 mM MgCl₂, about 320 mM to about 90 mM MgCl₂, about 30 mM to about 80 mM MgCl₂, about 30 mM to about 70 mM MgCl₂, about 30 mM to about 60 mM MgCl₂, about 30 mM to about 50 mM MgCl₂, about 30 mM to about 40 mM MgCl₂, about 40 mM to about 90 mM MgCl₂, about 40 mM to about 80 mM MgCl₂, about 40 mM to about 70 mM MgCl₂, about 40 mM to about 60 mM MgCl₂, about 40 mM to about 50 mM MgCl₂, about 50 mM to about 90 mM MgCl₂, about 50 mM to about 80 mM MgCl₂, about 50 mM to about 70 mM MgCl₂, about 50 mM to about 60 mM MgCl₂, about 60 mM to about 90 mM MgCl₂, about 60 mM to about 80 mM MgCl₂, about 60 mM to about 70 mM MgCl₂, about 70 mM to about 90 mM MgCl₂, about 70 mM to about 80 mM MgCl₂, or about 80 mM to about 90 mM MgCl₂. According to some embodiments of any of the aspects or embodiments herein, the ceDNA is closed-ended linear duplex DNA. According to some embodiments of any of the aspects or embodiments herein, the ceDNA comprises an expression cassette comprising a promoter sequence and a transgene. According to some embodiments, the ceDNA comprises expression cassette comprising a polyadenylation sequence. According to some embodiments of any of the aspects or embodiments herein, the ceDNA comprises at least one inverted terminal repeat (ITR) flanking either 5′ or 3′ end of said expression cassette. According to some embodiments, the expression cassette is flanked by two ITRs, wherein the two ITRs comprise one 5′ ITR and one 3′ ITR. According to some embodiments, the expression cassette is connected to an ITR at 3′ end (3′ ITR). According to some embodiments, the expression cassette is connected to an ITR at 5′ end (5′ ITR). According to some embodiments, at least one of 5′ ITR and 3′ ITR is a wild-type AAV ITR. According to some embodiments, at least one of 5′ ITR and 3′ ITR is a modified ITR. According to some embodiments, the ceDNA further comprises a spacer sequence between a 5′ ITR and the expression cassette. According to some embodiments, the ceDNA further comprises a spacer sequence between a 3′ ITR and the expression cassette. According to some embodiments, the spacer sequence is at least 5 base pairs long in length. According to some embodiments, the spacer sequence is 5 to 100 base pairs long in length. According to some embodiments, the spacer sequence is 5, 10, 15, 20, 25, 30, 35, 40, 45, 50, 55, 60, 65, 70, 75, 80, 85, 90, 95 or 100 base pairs long in length. According to some embodiments, the spacer sequence is 5 to 500 base pairs long in length. According to some embodiments, the spacer sequence is 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, or 495 base pairs long in length. According to some embodiments of any of the aspects or embodiments herein, the ceDNA has a nick or a gap. According to some embodiments, the ITR is an ITR derived from an AAV serotype, derived from an ITR of goose virus, derived from a B19 virus ITR, a wild-type ITR from a parvovirus. According to some embodiments, the AAV serotype is selected from the group comprising of AAV1, AAV2, AAV3, AAV4, AAV5, AAV6, AAV7, AAV8, AAV9, AAV10, AAV11 and AAV12. According to some embodiments, the ITR is a mutant ITR, and the ceDNA optionally comprises an additional ITR which differs from the first ITR. According to some embodiments, the ceDNA comprises two mutant ITRs in both 5′ and 3′ ends of the expression cassette, optionally wherein the two mutant ITRs are symmetric mutants. According to some embodiments of any of the aspects or embodiments herein, the ceDNA is a CELiD, DNA-based minicircle, a MIDGE, a ministering DNA, a dumbbell shaped linear duplex closed-ended DNA comprising two hairpin structures of ITRs in the 5′ and 3′ ends of an expression cassette, or a Doggybone™ DNA. According to some embodiments of any of the aspects or embodiments herein, the pharmaceutical composition further comprises a pharmaceutically acceptable excipient.

According to some aspects, the disclosure provides a method of treating a genetic disorder in a subject, the method comprising administering to the subject an effective amount of the pharmaceutical composition according to any of the aspects or embodiments herein. According to some embodiments, the subject is a human. According to some embodiments, the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson's disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS WA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, and Cathepsin A deficiency. According to some embodiments, the genetic disorder is Leber congenital amaurosis (LCA). According to some embodiments, the LCA is LCA10. According to some embodiments, the genetic disorder is Niemann-Pick disease. According to some embodiments, the genetic disorder is Stargardt macular dystrophy. According to some embodiments, the genetic disorder is glucose-6-phosphatase (G6Pase) deficiency (glycogen storage disease type I) or Pompe disease (glycogen storage disease type II). According to some embodiments, the genetic disorder is hemophilia A (Factor VIII deficiency). According to some embodiments, the genetic disorder is hemophilia B (Factor IX deficiency). According to some embodiments, the genetic disorder is hunter syndrome (Mucopolysaccharidosis II). According to some embodiments, the genetic disorder is cystic fibrosis. According to some embodiments, the genetic disorder is dystrophic epidermolysis bullosa (DEB). According to some embodiments, the genetic disorder is phenylketonuria (PKU). According to some embodiments, the genetic disorder is hyaluronidase deficiency. According to some embodiments of any of the aspects or embodiments herein, the method further comprises administering an immunosuppressant. According to some embodiments, the immunosuppressant is dexamethasone. According to some embodiments of any of the aspects or embodiments herein, the subject exhibits a diminished immune response level against the pharmaceutical composition, as compared to an immune response level observed with an LNP comprising MC3 as a main cationic lipid, wherein the immune response level against the pharmaceutical composition is at least 50% lower than the level observed with the LNP comprising MC3. According to some embodiments, the immune response is measured by detecting the levels of a pro-inflammatory cytokine or chemokine. According to some embodiments, the pro-inflammatory cytokine or chemokine is selected from the group consisting of IL-6, IFNα, IFNγ, IL-18, TNFα, IP-10, MCP-1, MIP1α, MIP1β, and RANTES. According to some embodiments, at least one of the pro-inflammatory cytokines is under a detectable level in serum of the subject at 6 hours after the administration of the pharmaceutical composition. According to some embodiments of any of the aspects or embodiments herein, the LNP comprising the SS-cleavable lipid and the closed-ended DNA (ceDNA) is not phagocytosed; or exhibits diminished phagocytic levels by at least 50% as compared to phagocytic levels of LNPs comprising MC3 as a main cationic lipid administered at a similar condition. According to some embodiments, the SS-cleavable lipid is ss-OP of Formula I. According to some embodiments, the LNP further comprises cholesterol and a PEG-lipid conjugate. According to some embodiments, the LNP further comprises a noncationic lipid. According to some embodiments, the noncationic lipid is selected from the group consisting of dioleoylphosphatidylcholine (DOPC), distearoylphosphatidylcholine (DSPC), and dioleoyl-phosphatidylethanolamine (DOPE). According to some embodiments, the LNP further comprises N-Acetylgalactosamine (GalNAc). According to some embodiments, the GalNAc is present in the LNP at a molar percentage of 0.5% of the total lipid.

According to another aspect, the disclosure provides a method of mitigating a complement response in a subject in need of treatment with a therapeutic nucleic acid, the method comprising administering to the subject an effective amount of a lipid nanoparticle LNP comprising therapeutic nucleic acid, ss-cleavable lipid, sterol, and polyethylene glycol (PEG) and N-Acetylgalactosamine (GalNAc). According to some embodiments, the subject is suffering from a genetic disorder. According to some embodiments, the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson's disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, and Cathepsin A deficiency. According to some embodiments, the therapeutic nucleic acid is selected from the group consisting of minigenes, plasmids, minicircles, small interfering RNA (siRNA), microRNA (miRNA), antisense oligonucleotides (ASO), ribozymes, ceDNA, ministring, Doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof. According to some embodiments, the ceDNA is selected from the group consisting of a CELiD, a MIDGE, a ministering DNA, a dumbbell shaped linear duplex closed-ended DNA comprising two hairpin structures of ITRs in the 5′ and 3′ ends of an expression cassette, or a Doggybone™ DNA, wherein the ceDNA is capsid free and linear duplex DNA. According to some embodiments, the PEG is 1-(monomethoxy-polyethyleneglycol)-2,3-dimyristoylglycerol (PEG-DMG). According to some embodiments, the PEG is present in the LNP at a molecular percentage of about 2% to 4%, e.g., about 2% to about 3.5%, about 2% to about 3%, about 2% to about 2.5%, about 2.5% to about 4%, about 2.5% to about 3.5%, abut 2.5% to about 3%, about 3% to about 4%, about 3.5% to about 4%, or about 2%, about 2.25%, about 2.5%, about 2.75%, about 3%, about 3.25%, about 3.5%, about 3.75%, or about 4%. According to some embodiments, the PEG is present in the LNP at a molecular percentage of about 3%. According to some embodiments, the LNP further comprises a non-cationic lipid. According to some embodiments, the non-cationic lipid is selected from the group consisting of dioleoylphosphatidylcholine (DOPC), distearoylphosphatidylcholine (DSPC), and dioleoyl-phosphatidylethanolamine (DOPE). According to some embodiments, the GalNAc is present in the LNP at a molar percentage of about 0.3 to 1% of the total lipid, e.g., about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 0.9%, about 0.4% to about 0.8%, about 0.4% to about 0.7%, about 0.4% to about 0.6%, about 0.4% to about 0.5%, about 0.5% to about 0.9%, about 0.5% to about 0.8%, about 0.5% to about 0.7%, about 0.5% to about 0.6%, about 0.6% to about 0.9%, about 0.6% to about 0.8%, about 0.6% to about 0.7%, about 0.7% to about 0.9%, about 0.7% to about 0.8%, about 0.8% to about 0.9% of the total lipid, or about 0.3%, about 0.4, about 0.5%, about 0.6%, about 0.7%, about 0.8%, about 0.9%, or about 1% of the total lipid. According to some embodiments, the GalNAc is present in the LNP at a molar percentage of about 0.5% of the total lipid.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A illustrates an exemplary structure of a ceDNA vector for expression of a transgene as disclosed herein, comprising asymmetric ITRs. In this embodiment, the exemplary ceDNA vector comprises an expression cassette containing CAG promoter, WPRE, and BGHpA. An open reading frame (ORF) encoding a transgene can be inserted into the cloning site (R3/R4) between the CAG promoter and WPRE. The expression cassette is flanked by two inverted terminal repeats (ITRs)—the wild-type AAV2 ITR on the upstream (5′-end) and the modified ITR on the downstream (3′-end) of the expression cassette, therefore the two ITRs flanking the expression cassette are asymmetric with respect to each other.

FIG. 1B illustrates an exemplary structure of a ceDNA vector for expression a transgene as disclosed herein comprising asymmetric ITRs with an expression cassette containing CAG promoter, WPRE, and BGHpA. An open reading frame (ORF) encoding the transgene can be inserted into the cloning site between CAG promoter and WPRE. The expression cassette is flanked by two inverted terminal repeats (ITRs)—a modified ITR on the upstream (5′-end) and a wild-type ITR on the downstream (3′-end) of the expression cassette.

FIG. 1C illustrates an exemplary structure of a ceDNA vector for expression of a transgene as disclosed herein comprising asymmetric ITRs, with an expression cassette containing an enhancer/promoter, the transgene, a post transcriptional element (WPRE), and a polyA signal. An open reading frame (ORF) allows insertion of transgene encoding a protein of interest, or therapeutic nucleic acid into the cloning site between CAG promoter and WPRE. The expression cassette is flanked by two inverted terminal repeats (ITRs) that are asymmetrical with respect to each other; a modified ITR on the upstream (5′-end) and a modified ITR on the downstream (3′-end) of the expression cassette, where the 5′ ITR and the 3′ITR are both modified ITRs but have different modifications (i.e., they do not have the same modifications).

FIG. 1D illustrates an exemplary structure of a ceDNA vector for expression of a transgene as disclosed herein, comprising symmetric modified ITRs, or substantially symmetrical modified ITRs as defined herein, with an expression cassette containing CAG promoter, WPRE, and BGHpA. An open reading frame (ORF) encoding the transgene is inserted into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1E illustrates an exemplary structure of a ceDNA vector for expression of a transgene as disclosed herein comprising symmetric modified ITRs, or substantially symmetrical modified ITRs as defined herein, with an expression cassette containing an enhancer/promoter, a transgene, a post transcriptional element (WPRE), and a polyA signal. An open reading frame (ORF) allows insertion of a transgene into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1F illustrates an exemplary structure of a ceDNA vector for expression of a transgene as disclosed herein, comprising symmetric WT-ITRs, or substantially symmetrical WT-ITRs as defined herein, with an expression cassette containing CAG promoter, WPRE, and BGHpA. An open reading frame (ORF) encoding a transgene is inserted into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 1G illustrates an exemplary structure of a ceDNA vector for expression of a transgene as disclosed herein, comprising symmetric modified ITRs, or substantially symmetrical modified ITRs as defined herein, with an expression cassette containing an enhancer/promoter, a transgene, a post transcriptional element (WPRE), and a polyA signal. An open reading frame (ORF) allows insertion of a transgene into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 2A provides the T-shaped stem-loop structure of a wild-type left ITR of with identification of A-A′ arm, B-B′ arm, C-C′ arm, two Rep binding sites (RBE and RBE′) and also shows the terminal resolution site (trs). The RBE contains a series of 4 duplex tetramers that are believed to interact with either Rep 78 or Rep 68. In addition, the RBE′ is also believed to interact with Rep complex assembled on the wild-type ITR or mutated ITR in the construct. The D and D′ regions contain transcription factor binding sites and other conserved structure. FIG. 2B shows proposed Rep-catalyzed nicking and ligating activities in a wild-type left ITR, including the T-shaped stem-loop structure of the wild-type left ITR of AAV2 with identification of A-A′ arm, B-B′ arm, C-C′ arm, two Rep Binding sites (RBE and RBE′) and also shows the terminal resolution site (trs), and the D and D′ region comprising several transcription factor binding sites and other conserved structure.

FIG. 3A provides the primary structure (polynucleotide sequence) (left) and the secondary structure (right) of the RBE-containing portions of the A-A′ arm, and the C-C′ and B-B′ arm of the wild type left AAV2 ITR. FIG. 3B shows an exemplary mutated ITR (also referred to as a modified ITR) sequence for the left ITR. Shown is the primary structure (left) and the predicted secondary structure (right) of the RBE portion of the A-A′ arm, the C arm and B-B′ arm of an exemplary mutated left ITR (ITR-1, left). FIG. 3C shows the primary structure (left) and the secondary structure (right) of the RBE-containing portion of the A-A′ loop, and the B-B′ and C-C′ arms of wild type right AAV2 ITR. FIG. 3D shows an exemplary right modified ITR. Shown is the primary structure (left) and the predicted secondary structure (right) of the RBE containing portion of the A-A′ arm, the B-B′ and the C arm of an exemplary mutant right ITR (ITR-1, right). Any combination of left and right ITR (e.g., AAV2 ITRs or other viral serotype or synthetic ITRs) can be used as taught herein. Each of FIGS. 3A-3D polynucleotide sequences refer to the sequence used in the plasmid or bacmid/baculovirus genome used to produce the ceDNA as described herein. Also included in each of FIGS. 3A-3D are corresponding ceDNA secondary structures inferred from the ceDNA vector configurations in the plasmid or bacmid/baculovirus genome and the predicted Gibbs free energy values.

FIG. 4A is a schematic illustrating an upstream process for making baculovirus infected insect cells (BIICs) that are useful in the production of a ceDNA vector for expression of a transgene as disclosed herein in the process described in the schematic in FIG. 4B. FIG. 4B is a schematic of an exemplary method of ceDNA production, and FIG. 4C illustrates a biochemical method and process to confirm ceDNA vector production. FIG. 4D and FIG. 4E are schematic illustrations describing a process for identifying the presence of ceDNA in DNA harvested from cell pellets obtained during the ceDNA production processes in FIG. 4B. FIG. 4D shows schematic expected bands for an exemplary ceDNA either left uncut or digested with a restriction endonuclease and then subjected to electrophoresis on either a native gel or a denaturing gel. The leftmost schematic is a native gel, and shows multiple bands suggesting that in its duplex and uncut form ceDNA exists in at least monomeric and dimeric states, visible as a faster-migrating smaller monomer and a slower-migrating dimer that is twice the size of the monomer. The schematic second from the left shows that when ceDNA is cut with a restriction endonuclease, the original bands are gone and faster-migrating (e.g., smaller) bands appear, corresponding to the expected fragment sizes remaining after the cleavage. Under denaturing conditions, the original duplex DNA is single-stranded and migrates as a species twice as large as observed on native gel because the complementary strands are covalently linked. Thus, in the second schematic from the right, the digested ceDNA shows a similar banding distribution to that observed on native gel, but the bands migrate as fragments twice the size of their native gel counterparts. The rightmost schematic shows that uncut ceDNA under denaturing conditions migrates as a single-stranded open circle, and thus the observed bands are twice the size of those observed under native conditions where the circle is not open. In this figure “kb” is used to indicate relative size of nucleotide molecules based, depending on context, on either nucleotide chain length (e.g., for the single stranded molecules observed in denaturing conditions) or number of basepairs (e.g., for the double-stranded molecules observed in native conditions). FIG. 4E shows DNA having a non-continuous structure. The ceDNA can be cut by a restriction endonuclease, having a single recognition site on the ceDNA vector, and generate two DNA fragments with different sizes (1 kb and 2 kb) in both neutral and denaturing conditions. FIG. 4E also shows a ceDNA having a linear and continuous structure. The ceDNA vector can be cut by the restriction endonuclease, and generate two DNA fragments that migrate as 1 kb and 2 kb in neutral conditions, but in denaturing conditions, the stands remain connected and produce single strands that migrate as 2 kb and 4 kb.

FIG. 5 is a graph that shows the efficiency of encapsulation, measured by determining unencapsulated ceDNA content (by measuring the fluorescence upon the addition of PicoGreen, (Thermo Scientific) to the LNP slurry (C_{free}) and comparing this value to the total ceDNA content obtained upon lysis of the LNPs by 1% Triton X-100 (Cₜₒₜₐₗ) where % encapsulation=(Cₜₒₜₐₗ−C_{free})/Cₜₒₜₐₗ×100).

FIG. 6A and FIG. 6B show efficiency of encapsulation measured by determining unencapsulated ceDNA content as described in FIG. 5 above. The effect of pH and salt condition on particle size and encapsulation rates were assessed. FIG. 6A shows effects on particle size and encapsulation rates at pH 4. FIG. 6B shows effects on particle size and encapsulation rates at pH 3. As shown in FIG. 6A and FIG. 6B, lipid particle size varied between approximately 70 nm to 120 nm in diameter. Encapsulation rates of 80% to 90% were achieved in these conditions.

FIG. 7 is a graph that depicts the effect of exemplary ceDNA LNPs described in Example 7 on body weight.

FIG. 8 is a graph that shows luciferase activity (total flux/photons per second) over time in each of the ceDNA LNP groups (MC3:PolyC; MC3:ceDNA-luc; ss-Paz3:PolyC; ss-Paz3: ceDNA-luc; ss-Paz3: ceDNA-luc+dexPalm; ss-Paz4:PolyC; ss-Paz4: ceDNA-luc; ss-OP3:PolyC; ss-OP3: ceDNA-luc; ss-OP4:PolyC; ss-OP4: ceDNA-luc).

FIG. 9 is a graph that depicts ceDNA expression (ceDNA copies per diploid genome) as detected in the liver qPCR, in mice treated with MC3 LNPs, ss-Paz3, ss-Paz4, ss-OP3 or ss-OP4 LNPs.

FIG. 10A and FIG. 10B show the effects of the ss-cleavable lipids in the ceDNA LNPs described in Example 7 on cytokine and chemokine levels (pg/ml) in the serum of mice.

FIG. 11 is a graph that shows luciferase activity (total flux/photons per second) over time in each of the ceDNA LNP groups (MC3:PolyC; MC3:ceDNA-luc; ss-OP4:PolyC; ss-OP4: ceDNA-luc).

FIG. 12A is a graph that depicts the effect on body weight of mice treated with exemplary ceDNA LNP (ss-OP4±0.5% GalNAc by lipid mol %) dosed at 0.5 mg/kg or 2.0 mg/kg. FIG. 12B shows the effects of the presence of GalNAc (as in ss-OP4:G, GalNAc present in 0.5% molar percentage of the total lipid weight) in the ss-OP4-ceDNA formulation on expression levels of ceDNA-luc.

FIG. 13 shows the effects of the ss-cleavable lipids in the ceDNA LNPs described in Example 8 on cytokine and chemokine levels (pg/ml) in the serum of the mice treated with ss-OP4 or ss-OP4 having GalNAc.

FIG. 14 shows a schematic of the phagocytosis assay for the ceDNA LNPs treated with 0.1% DiD (DiIC18(5); 1,1′-dioctadecyl-3,3,3′,3′-tetramethylindodicarbocyanine, 4-chlorobenzenesulfonate salt) lipophilic carbocyanine dye, where different concentrations of ceDNA (200 ng, 500 ng, 1 μg and 2 μg) were used in the MC3, MC3-5DSG or ss-OP4 LNPs, in the presence or absence of 10% human serum (+serum).

FIG. 15 shows images of ceDNA LNPs treated with 0.1% DiD (DiIC18(5); 1,1′-dioctadecyl-3,3,3′,3′-tetramethylindodicarbocyanine, 4-chlorobenzenesulfonate salt) lipophilic carbocyanine dye in which MC3, MC3-5DSG, or ss-OP4 lipid was used as LNP. Phagocytotic cells appear red, which can be seen as darker areas in the image.

FIG. 16 shows images of ceDNA LNPs treated with 0.1% DiD (DiIC18(5); 1,1′-dioctadecyl-3,3,3′,3′-tetramethylindodicarbocyanine, 4-chlorobenzenesulfonate salt) lipophilic carbocyanine dye. Phagocytotic cells appear red, which can be seen as darker areas in the image.

FIG. 17 is a graph showing quantification of phagocytosis (by red object count/% confluence) for ss-OP4, MC3-5DSG and MC3 LNPs.

FIG. 18A is a graph showing endosomal release or escape of ceDNA-ss-OP4 LNP at pH 7.4 and pH 6.0. FIG. 18B depicts quantification of ceDNA-luc in liver as measured by copy number in liver over copy number in spleen.

FIG. 19 shows the effects of ceDNA formulated in ss-OP4+GalNAc LNPs on the complement cascade proteins C3a and C5b9 (pg/ml) in the serum of test monkeys.

FIG. 20 shows the effects of ceDNA formulated in ss-OP4+GalNAc LNPs on INFα and INFβ cytokine levels (pg/ml) in the serum of test monkeys.

FIG. 21 shows the effects of ceDNA formulated in ss-OP4+GalNAc LNPs on INFγ and IL-1β cytokine levels (pg/ml) in the serum of test monkeys.

FIG. 22 shows the effects of ceDNA formulated in ss-OP4+GalNAc LNPs on IL-6 and IL-18 cytokine levels (pg/ml) in the serum of test monkeys.

FIG. 23 shows the effects of ceDNA formulated in ss-OP4+GalNAc LNPs on TNFα cytokine levels (pg/ml) in the serum of test monkeys.

FIG. 24 shows the effects of subretinal injection of ss-OP4/fLuc mRNA and ss-OP4/ceDNA-CpG minimized luciferase (ceDNA-luc) in rats.

FIG. 25 shows representative IVIS images of the effects of subretinal injection of ssOP4/fLuc mRNA and ssOP4/ceDNA-CpG minimized luciferase (eDNA-luc) in rat right (OD) and left (OS) eyes.

FIG. 26 shows the effects of the intravenous (IV) or subcutaneous (SC) administration of the ss-OP4-ceDNA formulation on expression levels of ceDNA-luc.

FIG. 27 shows the effects of the intravenous (IV) or subcutaneous (SC) administration of the ss-OP4-ceDNA formulation on cytokine and chemokine levels (mean concentration, pg/ml) in the serum of the mice.

### DETAILED DESCRIPTION

The present disclosure provides a lipid-based platform for delivering nucleic acids, e.g., therapeutic nucleic acids (TNAs), e.g., closed-ended DNA (ceDNA), which can move from the cytoplasm of the cell into the nucleus without viral capsid components. The immunogenicity associated with viral vector-based gene therapies has significantly limited the number of patients due to pre-existing background immunity and prevented the re-dosing of patients. Because of the lack of pre-existing immunity, the presently described therapeutic nucleic acid containing lipid particles (e.g., lipid nanoparticles) allow for additional doses of the therapeutic nucleic acid as necessary, and further expands patient access, including pediatric populations who may require a subsequent dose upon growth. Moreover, it is a finding of the present disclosure that the therapeutic nucleic acid containing lipid particles (e.g., lipid nanoparticles) comprising a cleavable lipid having one or more a tertiary amino groups, and a disulfide bond provide efficient delivery of the therapeutic nucleic acid with improved tolerability and safety profiles. Because the presently described therapeutic nucleic acid containing lipid particles (e.g., lipid nanoparticles) have no packaging constraints imposed by the space within the viral capsid, in theory, the only size limitation of the therapeutic nucleic acid containing lipid particles (e.g., lipid nanoparticles) resides in the DNA replication efficiency of the host cell.

As described and exemplified herein, the therapeutic nucleic acid can be closed-ended DNA (ceDNA). According to some embodiments, the therapeutic nucleic acid can be mRNA.

### I. Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6th Edition, published by Lippincott Williams & Wilkins, Philadelphia, Pa., USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al. Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used in this specification and the appended claims, the singular forms “a”, “an” and “the” include plural references unless the content clearly dictates otherwise.

The abbreviation, “e.g.” is derived from the Latin exempli gratia and is used herein to indicate a non-limiting example. Thus, the abbreviation “e.g.” is synonymous with the term “for example.”

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The term “consisting of” refers to compositions, methods, processes, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

As used herein the terms, “administration,” “administering” and variants thereof refers to introducing a composition or agent (e.g., nucleic acids, in particular ceDNA) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the phrase “anti-therapeutic nucleic acid immune response”, “anti-transfer vector immune response”, “immune response against a therapeutic nucleic acid”, “immune response against a transfer vector”, or the like is meant to refer to any undesired immune response against a therapeutic nucleic acid, viral or non-viral in its origin. In some embodiments, the undesired immune response is an antigen-specific immune response against the viral transfer vector itself. In some embodiments, the immune response is specific to the transfer vector which can be double stranded DNA, single stranded RNA, or double stranded RNA. In other embodiments, the immune response is specific to a sequence of the transfer vector. In other embodiments, the immune response is specific to the CpG content of the transfer vector.

As used herein, the term “aqueous solution” is meant to refer to a composition comprising in whole, or in part, water.

As used herein, the term “bases” includes purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the term “carrier” is meant to include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “ceDNA” is meant to refer to capsid-free closed-ended linear double stranded (ds) duplex DNA for non-viral gene transfer, synthetic or otherwise. According to some embodiments, the ceDNA is a closed-ended linear duplex (CELiD) CELiD DNA. According to some embodiments, the ceDNA is a DNA-based minicircle. According to some embodiments, the ceDNA is a minimalistic immunological-defined gene expression (MIDGE)-vector. According to some embodiments, the ceDNA is a ministering DNA. According to some embodiments, the ceDNA is a dumbbell shaped linear duplex closed-ended DNA comprising two hairpin structures of ITRs in the 5′ and 3′ ends of an expression cassette. According to some embodiments, the ceDNA is a Doggybone™ DNA. Detailed description of ceDNA is described in International application of PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International applications PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire contents of which is incorporated herein by reference.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As used herein, the term “ceDNA-bacmid” is meant to refer to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” is meant to refer to a baculovirus that comprises a ceDNA genome as an intermolecular duplex within the baculovirus genome.

As used herein, the terms “ceDNA-baculovirus infected insect cell” and “ceDNA-BIIC” are used interchangeably, and are meant to refer to an invertebrate host cell (including, but not limited to an insect cell (e.g., an Sf9 cell)) infected with a ceDNA-baculovirus.

As used herein, the term “ceDNA genome” is meant to refer to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the terms “DNA regulatory sequences,” “control elements,” and “regulatory elements,” are used interchangeably herein, and are meant to refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

As used herein, the phrase an “effective amount” or “therapeutically effective amount” of an active agent or therapeutic agent, such as a therapeutic nucleic acid, is an amount sufficient to produce the desired effect, e.g., inhibition of expression of a target sequence in comparison to the expression level detected in the absence of a therapeutic nucleic acid. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art.

As used herein, the term “exogenous” is meant to refer to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, as used herein, the term “endogenous” refers to a substance that is native to the biological system or cell.

As used herein, the term “expression” is meant to refer to the cellular processes involved in producing RNA and proteins and as appropriate, secreting proteins, including where applicable, but not limited to, for example, transcription, transcript processing, translation and protein folding, modification and processing. As used herein, the phrase “expression products” include RNA transcribed from a gene (e.g., transgene), and polypeptides obtained by translation of mRNA transcribed from a gene.

As used herein, the term “expression vector” is meant to refer to a vector that directs expression of an RNA or polypeptide from sequences linked to transcriptional regulatory sequences on the vector. The sequences expressed will often, but not necessarily, be heterologous to the host cell. An expression vector may comprise additional elements, for example, the expression vector may have two replication systems, thus allowing it to be maintained in two organisms, for example in human cells for expression and in a prokaryotic host for cloning and amplification. the expression vector may be a recombinant vector.

As used herein, the terms “expression cassette” and “expression unit” are used interchangeably, and meant to refer to a heterologous DNA sequence that is operably linked to a promoter or other DNA regulatory sequence sufficient to direct transcription of a transgene of a DNA vector, e.g., synthetic AAV vector. Suitable promoters include, for example, tissue specific promoters. Promoters can also be of AAV origin.

As used herein, the term “terminal repeat” or “TR” includes any viral or non-viral terminal repeat or synthetic sequence that comprises at least one minimal required origin of replication and a region comprising a palindromic hairpin structure. A Rep-binding sequence (“RBS” or also referred to as Rep-binding element (RBE)) and a terminal resolution site (“TRS”) together constitute a “minimal required origin of replication” for an AAV and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs plays a critical role in mediating replication, viral particle and DNA packaging, DNA integration and genome and provirus rescue. TRs that are not inverse complement (palindromic) across their full length can still perform the traditional functions of ITRs, and thus, the term ITR is used to refer to a TR in an viral or non-viral AAV vector that is capable of mediating replication of in the host cell. It will be understood by one of ordinary skill in the art that in a complex AAV vector configurations more than two ITRs or asymmetric ITR pairs may be present.

The “ITR” can be artificially synthesized using a set of oligonucleotides comprising one or more desirable functional sequences (e.g., palindromic sequence, RBS). The ITR sequence can be an AAV ITR, an artificial non-AAV ITR, or an ITR physically derived from a viral AAV ITR (e.g., ITR fragments removed from a viral genome). For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvoviridae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. Typically, ITR sequences can be derived not only from AAV, but also from Parvovirus, lentivirus, goose virus, B19, in the configurations of wildtype, “doggy bone” and “dumbbell shape”, symmetrical or even asymmetrical ITR orientation. Although the ITRs are typically present in both 5′ and 3′ ends of an AAV vector, ITR can be present in only one of end of the linear vector. For example, the ITR can be present on the 5′ end only. Some other cases, the ITR can be present on the 3′ end only in synthetic AAV vector. For convenience herein, an ITR located 5′ to (“upstream of”) an expression cassette in a synthetic AAV vector is referred to as a “5′ ITR” or a “left ITR”, and an ITR located 3′ to (“downstream of”) an expression cassette in a vector or synthetic AAV is referred to as a “3′ ITR” or a “right ITR”.

As used herein, a “wild-type ITR” or “WT-ITR” refers to the sequence of a naturally occurring ITR sequence in an AAV genome or other dependovirus that remains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompasses for use herein include WT-ITR sequences as result of naturally occurring changes (e.g., a replication error).

As used herein, the term “substantially symmetrical WT-ITRs” or a “substantially symmetrical WT-ITR pair” refers to a pair of WT-ITRs within a synthetic AAV vector that are both wild type ITRs that have an inverse complement sequence across their entire length. For example, an ITR can be considered to be a wild-type sequence, even if it has one or more nucleotides that deviate from the canonical naturally occurring canonical sequence, so long as the changes do not affect the physical and functional properties and overall three-dimensional structure of the sequence (secondary and tertiary structures). In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably and refer to an ITR with a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single synthetic AAV genome that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single stranded AAV genome that are wild-type or mutated (e.g., modified relative to wild-type) dependoviral ITR sequences and are inverse complements across their full length. In one non-limiting example, both ITRs are wild type ITRs sequences from AAV2. In another example, neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a synthetic AAV vector is referred to as a “5′ ITR” or a “left ITR”, and an ITR located 3′ to (downstream of) an expression cassette in a synthetic AAV vector is referred to as a “3′ ITR” or a “right ITR”.

As used herein, the terms “substantially symmetrical modified-ITRs” or a “substantially symmetrical mod-ITR pair” refers to a pair of modified-ITRs within a synthetic AAV that are both that have an inverse complement sequence across their entire length. For example, a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

As used herein, the term “flanking” is meant to refer to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear single strand synthetic AAV vector.

As used herein, the term “gap” is meant to refer to a discontinued portion of synthetic DNA vector of the present invention, creating a stretch of single stranded DNA portion in otherwise double stranded ceDNA. The gap can be 1 base-pair to 100 base-pair long in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 bp long in length. Exemplified gaps in the present disclosure can be 1 bp to 10 bp long, 1 to 20 bp long, 1 to 30 bp long in length.

As used herein, the term “nick” refers to a discontinuity in a double stranded DNA molecule where there is no phosphodiester bond between adjacent nucleotides of one strand typically through damage or enzyme action. It is understood that one or more nicks allow for the release of torsion in the strand during DNA replication and that nicks are also thought to play a role in facilitating binding of transcriptional machinery.

As used herein, the term “neDNA”, “nicked ceDNA” refers to a closed-ended DNA having a nick or a gap of 1-100 base pairs a stem region or spacer region upstream of an open reading frame (e.g., a promoter and transgene to be expressed).

As used herein, the term “gene” is used broadly to refer to any segment of nucleic acid associated with expression of a given RNA or protein, in vitro or in vivo. Thus, genes include regions encoding expressed RNAs (which typically include polypeptide coding sequences) and, often, the regulatory sequences required for their expression. Genes can be obtained from a variety of sources, including cloning from a source of interest or synthesizing from known or predicted sequence information, and may include sequences designed to have specifically desired parameters.

As used herein, the term “gene delivery” means a process by which foreign DNA is transferred to host cells for applications of gene therapy.

As used herein, the phrase “genetic disease” or “genetic disorder” is meant to refer to a disease, partially or completely, directly or indirectly, caused by one or more abnormalities in the genome, including and especially a condition that is present from birth. The abnormality may be a mutation, an insertion or a deletion in a gene. The abnormality may affect the coding sequence of the gene or its regulatory sequence.

As used herein, the term s “heterologous nucleotide sequence” and “transgene” are used interchangeably and refer to a nucleic acid of interest (other than a nucleic acid encoding a capsid polypeptide) that is incorporated into and may be delivered and expressed by a vector, such as ceDNA vector, as disclosed herein. A heterologous nucleic acid sequence may be linked to a naturally occurring nucleic acid sequence (or a variant thereof) (e.g., by genetic engineering) to generate a chimeric nucleotide sequence encoding a chimeric polypeptide. A heterologous nucleic acid sequence may be linked to a variant polypeptide (e.g., by genetic engineering) to generate a nucleotide sequence encoding a fusion variant polypeptide.

As used herein, the term “homology” or “homologous” is meant to refer to the percentage of nucleotide residues in the homology arm that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm of a repair template, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

As used herein, the term “host cell” refers to any cell type that is susceptible to transformation, transfection, transduction, and the like with nucleic acid therapeutics of the present disclosure. As non-limiting examples, a host cell can be an isolated primary cell, pluripotent stem cells, CD34⁺ cells, induced pluripotent stem cells, or any of a number of immortalized cell lines (e.g., HepG2 cells). Alternatively, a host cell can be an in situ or in vivo cell in a tissue, organ or organism. Furthermore, a host cell can be a target cell of, for example, a mammalian subject (e.g., human patient in need of gene therapy).

As used herein, an “inducible promoter” is meant to refer to one that is characterized by initiating or enhancing transcriptional activity when in the presence of, influenced by, or contacted by an inducer or inducing agent. An “inducer” or “inducing agent,” as used herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

As used herein, the term “in vitro” is meant to refer to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

As used herein, the term “in vivo” is meant to refer to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others.

As used herein, the term “lipid” is meant to refer to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

In one embodiment, the lipid compositions comprise one or more tertiary amino groups, one or more phenyl ester bonds, and a disulfide bond.

As used herein, the term “lipid conjugate” is meant to refer to a conjugated lipid that inhibits aggregation of lipid particles (e.g., lipid nanoparticles). Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

As used herein, the term “lipid encapsulated” is meant to refer to a lipid particle that provides an active agent or therapeutic agent, such as a nucleic acid (e.g., a ceDNA), with full encapsulation, partial encapsulation, or both. In a preferred embodiment, the nucleic acid is fully encapsulated in the lipid particle (e.g., to form a nucleic acid containing lipid particle).

As used herein, the terms “lipid particle” or “lipid nanoparticle” is meant to refer to a lipid formulation that can be used to deliver a therapeutic agent such as nucleic acid therapeutics to a target site of interest (e.g., cell, tissue, organ, and the like). In one embodiment, the lipid particle of the invention is a nucleic acid containing lipid particle, which is typically formed from a cationic lipid, a non-cationic lipid, and optionally a conjugated lipid that prevents aggregation of the particle. In other preferred embodiments, a therapeutic agent such as a therapeutic nucleic acid may be encapsulated in the lipid portion of the particle, thereby protecting it from enzymatic degradation. In one embodiment, the lipid particle comprises a nucleic acid (e.g., ceDNA) and a lipid comprising one or more a tertiary amino groups, one or more phenyl ester bonds and a disulfide bond.

The lipid particles of the invention typically have a mean diameter of from about 20 nm to about 120 nm, about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm.

As used herein, the term “cationic lipid” refers to any lipid that is positively charged at physiological pH. The cationic lipid in the lipid particles may comprise, e.g., one or more cationic lipids such as 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)[1,3]-dioxolane (DLin-K-C2-DMA), 2,2-dilinoleyl-4-dimethylaminomethyl-[1,3]-dioxolane (DLin-K-DMA), “SS-cleavable lipid”, or a mixture thereof. In some embodiments, a cationic lipid is also an ionizable lipid, i.e., an ionizable cationic lipid.

As used herein, the term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

As used herein, the term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N-N-dialkylamino, 1,2-diacyloxy-3-aminopropane, and 1,2-dialkyl-3-aminopropane.

As used herein, the term “ionizable lipid” is meant to refer to a lipid, e.g., cationic lipid, having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. In some embodiments, ionizable lipid may include “cleavable lipid” or “SS-cleavable lipid”.

As used herein, the term “neutral lipid” is meant to refer to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

As used herein, the term “non-cationic lipid” is meant to refer to any amphipathic lipid as well as any other neutral lipid or anionic lipid.

As used herein, the term “cleavable lipid” or “SS-cleavable lipid” refers to a lipid comprising a disulfide bond cleavable unit. Cleavable lipids may include cleavable disulfide bond (“ss”) containing lipid-like materials that comprise a pH-sensitive tertiary amine and self-degradable phenyl ester. For example, a SS-cleavable lipid can be an ss-OP lipid (COATSOME® SS-OP), an ss-M lipid (COATSOME® SS-M), an ss-E lipid (COATSOME® SS-E), an ss-EC lipid (COATSOME® SS-EC), an ss-LC lipid (COATSOME® SS-LC), an ss-OC lipid (COATSOME® SS-OC), and an ss-PalmE lipid (see, for example, Formulae I-IV), or a lipid described by Togashi et al., (2018) Journal of Controlled Release “A hepatic pDNA delivery system based on an intracellular environment sensitive vitamin E-scaffold lipid-like material with the aid of an anti-inflammatory drug” 279:262-270. Additional examples of cleavable lipids are described in U.S. Pat. Nos. 9,708,628, and 10,385,030, the entire contents of which are incorporated herein by reference. In one embodiment, cleavable lipids comprise a tertiary amine, which responds to an acidic compartment, e.g., an endosome or lysosome for membrane destabilization and a disulfide bond that can be cleaved in a reducing environment, such as the cytoplasm. In one embodiment, a cleavable lipid is a cationic lipid. In one embodiment, a cleavable lipid is an ionizable cationic lipid. Cleavable lipids are described in more detail herein.

As used herein, the term “organic lipid solution” is meant to refer to a composition comprising in whole, or in part, an organic solvent having a lipid.

As used herein, the term “liposome” is meant to refer to lipid molecules assembled in a spherical configuration encapsulating an interior aqueous volume that is segregated from an aqueous exterior. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “local delivery” is meant to refer to delivery of an active agent such as an interfering RNA (e.g., siRNA) directly to a target site within an organism. For example, an agent can be locally delivered by direct injection into a disease site such as a tumor or other target site such as a site of inflammation or a target organ such as the liver, heart, pancreas, kidney, and the like.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), Doggybone™ DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, Doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

An “inhibitory polynucleotide” as used herein refers to a DNA or RNA molecule that reduces or prevents expression (transcription or translation) of a second (target) polynucleotide. Inhibitory polynucleotides include antisense polynucleotides, ribozymes, and external guide sequences. The term “inhibitory polynucleotide” further includes DNA and RNA molecules, e.g., RNAi that encode the actual inhibitory species, such as DNA molecules that encode ribozymes.

As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “interfering RNA” or “RNAi” or “interfering RNA sequence” includes single-stranded RNA (e.g., mature miRNA, ssRNAi oligonucleotides, ssDNAi oligonucleotides), double-stranded RNA (i.e., duplex RNA such as siRNA, Dicer-substrate dsRNA, shRNA, aiRNA, or pre-miRNA), a DNA-RNA hybrid (see, e.g., PCT Publication No. WO 2004/078941), or a DNA-DNA hybrid (see, e.g., PCT Publication No. WO 2004/104199) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. Preferably, the interfering RNA molecules are chemically synthesized. The disclosures of each of the above patent documents are herein incorporated by reference in their entirety for all purposes. The term “RNAi” can include both gene silencing RNAi molecules, and also RNAi effector molecules which activate the expression of a gene. In some embodiments RNAi agents which serve to inhibit or gene silence are useful in the methods, kits and compositions disclosed herein, e.g., to inhibit the immune response.

Interfering RNA includes “small-interfering RNA” or “siRNA,” e.g., interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 18-22, 19-20, or 19-21 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule. As used herein, the term “siRNA” includes RNA-RNA duplexes as well as DNA-RNA hybrids (see, e.g., PCT Publication No. WO 2004/078941).

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to an uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

As used herein, “nucleotides” contain a sugar deoxyribose (DNA) or ribose (RNA), a base, and a phosphate group. Nucleotides are linked together through the phosphate groups.

As used herein, “operably linked” is meant to refer to a juxtaposition wherein the components so described are in a relationship permitting them to function in their intended manner. For instance, a promoter is operably linked to a coding sequence if the promoter affects its transcription or expression. A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered compound.

As used herein, the term “promoter” is meant to refer to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the synthetic AAV vectors disclosed herein. A promoter sequence may be bounded at its 3′ terminus by the transcription initiation site and extends upstream (5′ direction) to include the minimum number of bases or elements necessary to initiate transcription at levels detectable above background.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence. In some embodiments, a coding nucleic acid segment is positioned under the control of a “recombinant promoter” or “heterologous promoter,” both of which refer to a promoter that is not normally associated with the encoded nucleic acid sequence that it is operably linked to in its natural environment. Similarly, a “recombinant or heterologous enhancer” refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference in its entirety). Furthermore, it is contemplated that control sequences that direct transcription and/or expression of sequences within non-nuclear organelles such as mitochondria, chloroplasts, and the like, can be employed as well.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence Enhancers can be positioned up to 1,000,000 base pars upstream of the gene start site or downstream of the gene start site that they regulate. An enhancer can be positioned within an intronic region, or in the exonic region of an unrelated gene.

As used herein, the terms “Rep binding site” (“RBS”) and “Rep binding element” (“RBE”) are used interchangeably and are meant to refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are well known in the art, and include, for example, 5′-GCGCGCTCGCTCGCTC-3′ (SEQ ID NO: 1), an RBS sequence identified in AAV2. Any known RBS sequence may be used in the embodiments of the invention, including other known AAV RBS sequences and other naturally known or synthetic RBS sequences. Without being bound by theory it is thought that he nuclease domain of a Rep protein binds to the duplex nucleotide sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 1). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the phrase “recombinant vector” is meant to refer to a vector that includes a heterologous nucleic acid sequence, or “transgene” that is capable of expression in vivo. It is to be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “reporter” is meant to refer to a protein that can be used to provide a detectable read-out. A reporter generally produces a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest. Promoters are regions of nucleic acid that initiate transcription of a particular gene. Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to, homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

As used herein, the terms “sense” and “antisense” are meant to refer to the orientation of the structural element on the polynucleotide. The sense and antisense versions of an element are the reverse complement of each other.

As used herein, the term “sequence identity” is meant to refer to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

As used herein, the term “spacer region” is meant to refer to an intervening sequence that separates functional elements in a vector or genome. In some embodiments, AAV spacer regions keep two functional elements at a desired distance for optimal functionality. In some embodiments, the spacer regions provide or add to the genetic stability of the vector or genome. In some embodiments, spacer regions facilitate ready genetic manipulation of the genome by providing a convenient location for cloning sites and a gap of design number of base pair. For example, in certain aspects, an oligonucleotide “polylinker” or “poly cloning site” containing several restriction endonuclease sites, or a non-open reading frame sequence designed to have no known protein (e.g., transcription factor) binding sites can be positioned in the vector or genome to separate the cis—acting factors, e.g., inserting a 6mer, 12mer, 18mer, 24mer, 48mer, 86mer, 176mer, etc.

As used herein, the term “subject” is meant to refer to a human or animal, to whom treatment, including prophylactic treatment, with the therapeutic nucleic acid according to the present invention, is provided. Usually the animal is a vertebrate such as, but not limited to a primate, rodent, domestic animal or game animal. Primates include but are not limited to, chimpanzees, cynomolgus monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the phrase “subject in need” refers to a subject that (i) will be administered a ceDNA lipid particle (or pharmaceutical composition comprising a ceDNA lipid particle) according to the described invention, (ii) is receiving a ceDNA lipid particle (or pharmaceutical composition comprising aceDNA lipid particle) according to the described invention; or (iii) has received a ceDNA lipid particle (or pharmaceutical composition comprising a ceDNA lipid particle) according to the described invention, unless the context and usage of the phrase indicates otherwise.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the terms “synthetic AAV vector” and “synthetic production of AAV vector” are meant to refer to an AAV vector and synthetic production methods thereof in an entirely cell-free environment.

As used herein, the term “systemic delivery” is meant to refer to delivery of lipid particles that leads to a broad biodistribution of an active agent such as an interfering RNA (e.g., siRNA) within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles (e.g., lipid nanoparticles) can be by any means known in the art including, for example, intravenous, subcutaneous, and intraperitoneal. In a preferred embodiment, systemic delivery of lipid particles (e.g., lipid nanoparticles) is by intravenous delivery.

As used herein, the terms “terminal resolution site” and “trs” are used interchangeably herein and are meant to refer to a region at which Rep forms a tyrosine-phosphodiester bond with the 5′ thymidine generating a 3′-OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′, the hexanucleotide sequence identified in AAV2. Any known TRS sequence may be used in the embodiments of the invention, including other known AAV TRS sequences and other naturally known or synthetic TRS sequences such as AGTT, GGTTGG, AGTTGG, AGTTGA, and other motifs such as RRTTRR.

As used herein, the terms “therapeutic amount”, “therapeutically effective amount”, an “amount effective”, or “pharmaceutically effective amount” of an active agent (e.g. a ceDNA lipid particle as described herein) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the terms “vector” or “expression vector” are meant to refer to a replicon, such as plasmid, bacmid, phage, virus, virion, or cosmid, to which another DNA segment, i.e. an “insert” “transgene” or “expression cassette”, may be attached so as to bring about the expression or replication of the attached segment (“expression cassette”) in a cell. A vector can be a nucleic acid construct designed for delivery to a host cell or for transfer between different host cells. As used herein, a vector can be viral or non-viral in origin in the final form. However, for the purpose of the present disclosure, a “vector” generally refers to synthetic AAV vector or a nicked ceDNA vector. Accordingly, the term “vector” encompasses any genetic element that is capable of replication when associated with the proper control elements and that can transfer gene sequences to cells. In some embodiments, a vector can be a recombinant vector or an expression vector.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this invention is not limited in any manner to the particular methodology, protocols, and reagents, etc., described herein and as such can vary. The terminology used herein is for the purpose of describing particular embodiments only and is not intended to limit the scope of the present invention, which is defined solely by the claims.

### II. Cleavable Lipids

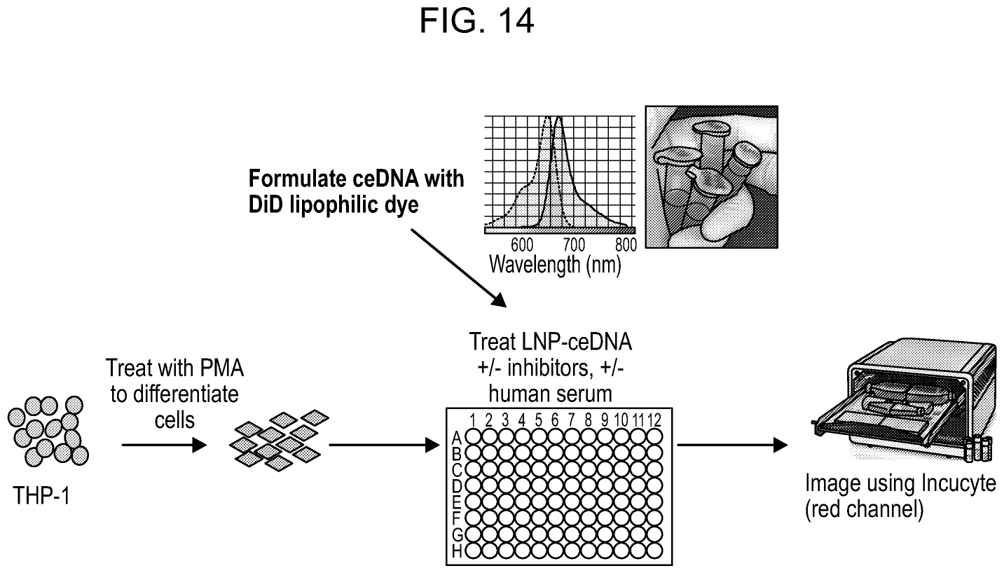

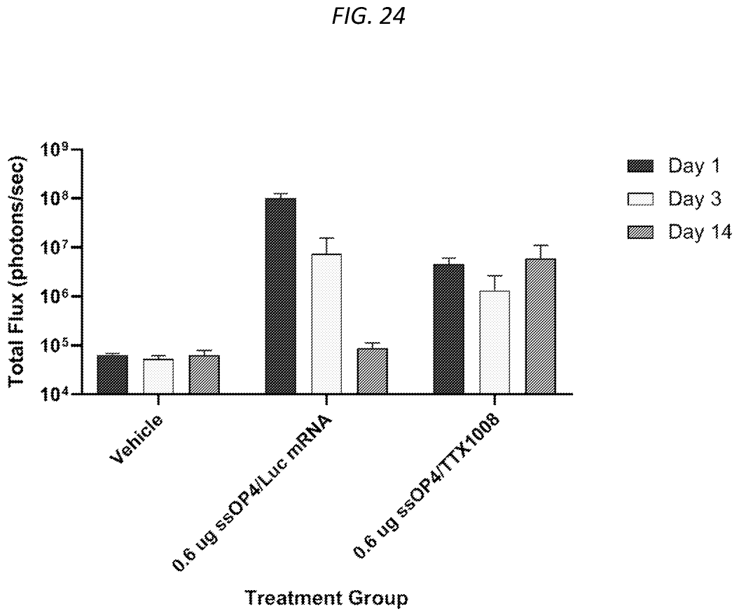

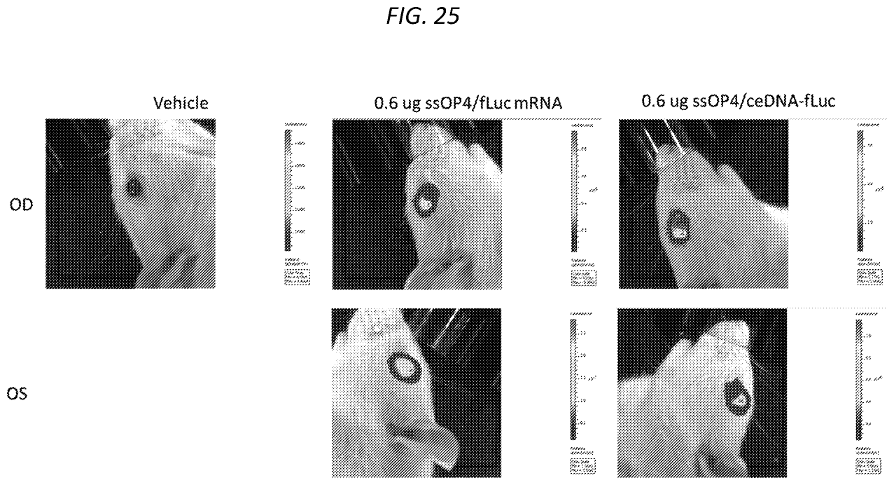

Provided herein are pharmaceutical compositions comprising a cleavable lipid and a capsid free, non-viral vector (e.g., ceDNA) that can be used to deliver the capsid-free, non-viral DNA vector to a target site of interest (e.g., cell, tissue, organ, and the like). As used herein, the term “cleavable lipid” refers to a cationic lipid comprising a disulfide bond (“SS”) cleavable unit. In one embodiment, SS-cleavable lipids comprise a tertiary amine, which responds to an acidic compartment (e.g., an endosome or lysosome) for membrane destabilization and a disulfide bond that can cleave in a reductive environment (e.g., the cytoplasm). SS-cleavable lipids may include SS-cleavable and pH-activated lipid-like materials, such as ss-OP lipids, ssPalm lipids, ss-M lipids, ss-E lipids, ss-EC lipids, ss-LC lipids and ss-OC lipids, etc. As demonstrated herein, ceDNA lipid particles (e.g., lipid nanoparticles) comprising a cleavable lipid provide more efficient delivery of ceDNA to target cells, including, e.g., hepatic cells. As reported by the present disclosure, a ceDNA particle comprising ceDNA and a cleavable lipid resulted in fewer ceDNA copies in the liver with equivalent luciferase expression compared to other lipids, e.g., MC3. Indeed, a synergistic effect between the ceDNA and cleavable lipid is observed, which minimizes the phagocytic effect (see, for example, FIGS. 14-17) while increasing ceDNA expression by up to 4,000-fold as compared to other lipids, e.g., MC3. As also reported by the present disclosure, a lipid formulation comprising mRNA and a cleavable lipid resulted in increased transgene expression compared to vehicle control up to 3 days after subretinal injection in rats (FIG. 24 and FIG. 25). Accordingly, the lipid particles (e.g., ceDNA lipid particles or mRNA lipid particles) described herein can advantageously be used to increase delivery of nucleic acids (e.g., ceDNA or mRNA) to target cells/tissues as compared to other conventional lipids with minimal or no phagocytic effect. Thus, the lipid particles (e.g., ceDNA lipid particles, or mRNA lipid particles) described herein provided enhanced nucleic acid delivery compared to conventional lipid nanoparticles known in the art. Although the mechanism has not yet been determined, and without being bound by theory, it is thought that the lipid particles (e.g., ceDNA lipid particles or mRNA lipid particles) comprising a cleavable lipid provide improved delivery to hepatocytes escaping phagocytosis. Another advantage of the ceDNA containing lipid particles comprising a cleavable lipid described herein is that they exhibit superior tolerability as compared to other lipid nanoparticles, e.g., MC3, in vivo.

In one embodiment, a cleavable lipid may comprise three components: an amine head group, a linker group, and a hydrophobic tail(s). In one embodiment, the cleavable lipid comprises one or more phenyl ester bonds, one of more tertiary amino groups, and a disulfide bond. The tertiary amine groups provide pH responsiveness and induce endosomal escape, the phenyl ester bonds enhance the degradability of the structure (self-degradability) and the disulfide bond cleaves in a reductive environment.

In one embodiment, the cleavable lipid is an ss-OP lipid. In one embodiment, an ss-OP lipid comprises the structure shown in Formula I below:

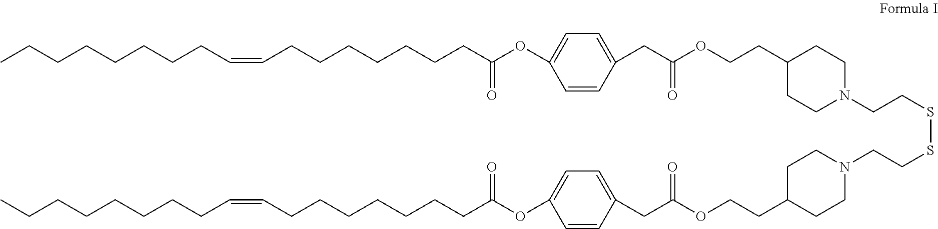 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In one embodiment, the SS-cleavable lipid is an SS-cleavable and pH-activated lipid-like material (ssPalm). ssPalm lipids are well known in the art. For example, see Togashi et al., Journal of Controlled Release, 279 (2018) 262-270, the entire contents of which are incorporated herein by reference. In one embodiment, the ssPalm is an ssPalmM lipid comprising the structure of Formula II.

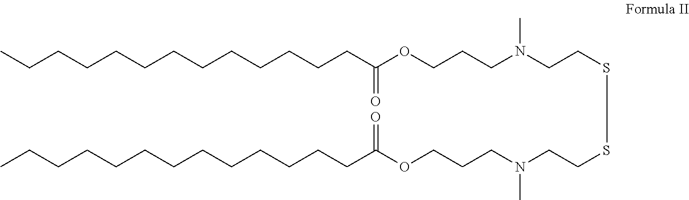 [SMILES: CCCCCCCCCCCCCC(=O)OCCCN(C)CCSCSCCN(C)CCCOC(=O)CCCCCCCCCCCCC]

In one embodiment, the ssPalmE lipid is a ssPalmE-P4-C2 lipid, comprising the structure of Formula III.

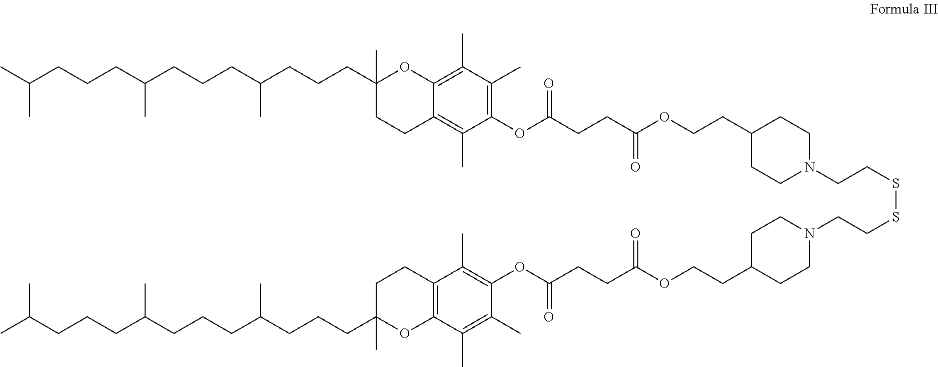 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the ssPalmE lipid is a ssPalmE-Paz4-C2 lipid, comprising the structure of Formula IV.

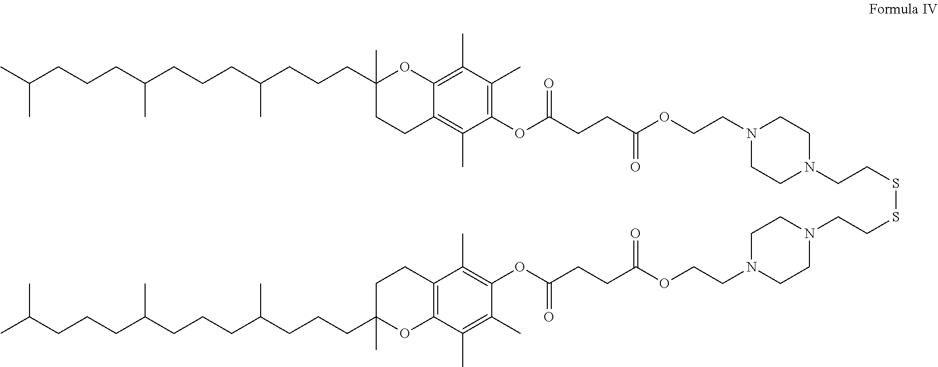 [SMILES: Cc1c(C)c2c(c(C)c1OC(=O)CCC(=O)OCCN1CCN(CCSSCCN3CCN(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-M lipid. In one embodiment, an ss-M lipid comprises the structure shown in Formula V below:

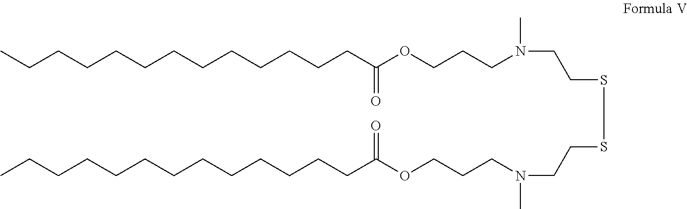 [SMILES: CCCCCCCCCCCCCC(=O)OCCCN(C)CCSCSCCN(C)CCCOC(=O)CCCCCCCCCCCCC]

In one embodiment, the cleavable lipid is an ss-E lipid. In one embodiment, an ss-E lipid comprises the structure shown in Formula VI below:

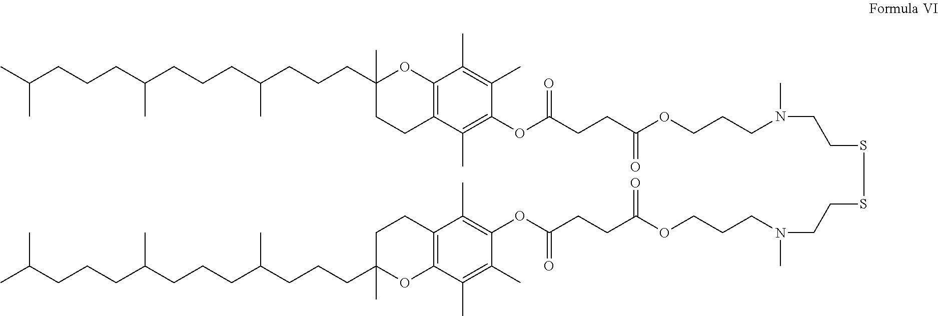 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2]

In one embodiment, the cleavable lipid is an ss-EC lipid. In one embodiment, an ss-EC lipid comprises the structure shown in Formula VII below:

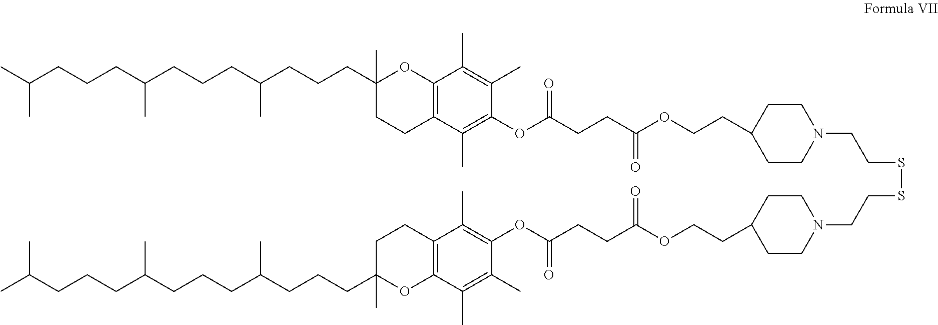 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-LC lipid. In one embodiment, an ss-LC lipid comprises the structure shown in Formula VIII below:

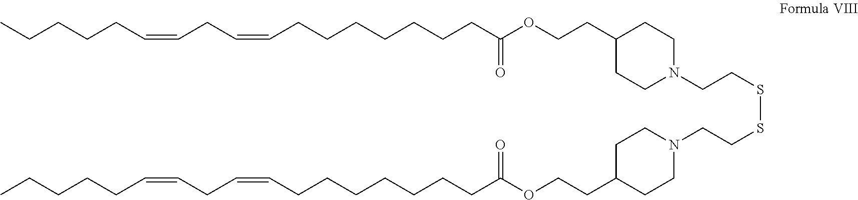 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1]

In one embodiment, the cleavable lipid is an ss-OC lipid. In one embodiment, an ss-OC lipid comprises the structure shown in Formula IX below:

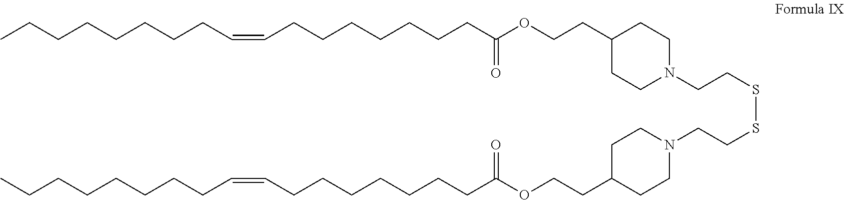 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

In one embodiment, a lipid particle (e.g., lipid nanoparticle) formulation is made and loaded with ceDNA obtained by the process as disclosed in International Application PCT/US2018/050042, filed on Sep. 7, 2018, which is incorporated by reference in its entirety herein. This can be accomplished by high energy mixing of ethanolic lipids with aqueous ceDNA at low pH which protonates the lipid and provides favorable energetics for ceDNA/lipid association and nucleation of particles. The particles can be further stabilized through aqueous dilution and removal of the organic solvent. The particles can be concentrated to the desired level. In one embodiment, the disclosure provides a ceDNA lipid particle comprising a lipid of Formula I prepared by a process as described in Example 6.

Generally, the lipid particles (e.g., lipid nanoparticles) are prepared at a total lipid to ceDNA (mass or weight) ratio of from about 10:1 to 60:1. In some embodiments, the lipid to ceDNA ratio (mass/mass ratio; w/w ratio) can be in the range of from about 1:1 to about 60:1, from about 1:1 to about 55:1, from about 1:1 to about 50:1, from about 1:1 to about 45:1, from about 1:1 to about 40:1, from about 1:1 to about 35:1, from about 1:1 to about 30:1, from about 1:1 to about 25:1, from about 10:1 to about 14:1, from about 3:1 to about 15:1, from about 4:1 to about 10:1, from about 5:1 to about 9:1, about 6:1 to about 9:1; from about 30:1 to about 60:1. According to some embodiments, the lipid particles (e.g., lipid nanoparticles) are prepared at a ceDNA (mass or weight) to total lipid ratio of about 60:1. According to some embodiments, the lipid particles (e.g., lipid nanoparticles) are prepared at a ceDNA (mass or weight) to total lipid ratio of about 30:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

In some embodiments, the lipid nanoparticle comprises an agent for condensing and/or encapsulating nucleic acid cargo, such as ceDNA. Such an agent is also referred to as a condensing or encapsulating agent herein. Without limitations, any compound known in the art for condensing and/or encapsulating nucleic acids can be used as long as it is non-fusogenic. In other words, an agent capable of condensing and/or encapsulating the nucleic acid cargo, such as ceDNA, but having little or no fusogenic activity. Without wishing to be bound by a theory, a condensing agent may have some fusogenic activity when not condensing/encapsulating a nucleic acid, such as ceDNA, but a nucleic acid encapsulating lipid nanoparticle formed with said condensing agent can be non-fusogenic.

Generally, the cationic lipid is typically employed to condense the nucleic acid cargo, e.g., ceDNA at low pH and to drive membrane association and fusogenicity. Generally, catonic lipids are lipids comprising at least one amino group that is positively charged or becomes protonated under acidic conditions, for example at pH of 6.5 or lower. Cationic lipids may also be ionizable lipids, e.g., ionizable cationic lipids. By a “non-fusogenic cationic lipid” is meant a cationic lipid that can condense and/or encapsulate the nucleic acid cargo, such as ceDNA, but does not have, or has very little, fusogenic activity.

In one embodiment, the cationic lipid can comprise 20-90% (mol) of the total lipid present in the lipid particles (e.g., lipid nanoparticles). For example, cationic lipid molar content can be 20-70% (mol), 30-60% (mol), 40-60% (mol), 40-55% (mol) or 45-55% (mol) of the total lipid present in the lipid particle (e.g., lipid nanoparticles). In some embodiments, cationic lipid comprises from about 50 mol % to about 90 mol % of the total lipid present in the lipid particles (e.g., lipid nanoparticles).

In one embodiment, the SS-cleavable lipid is not MC3 (6Z,9Z,28Z,31Z)-heptatriaconta-6,9,28,31-tetraen-19-yl-4-(dimethylamino)butanoate (DLin-MC3-DMA or MC3). DLin-MC3-DMA is described in Jayaraman et al., Angew. Chem. Int. Ed Engl. (2012), 51(34): 8529-8533, the contents of which is incorporated herein by reference in its entirety. The structure of D-Lin-MC3-DMA (MC3) is shown below as Formula X:

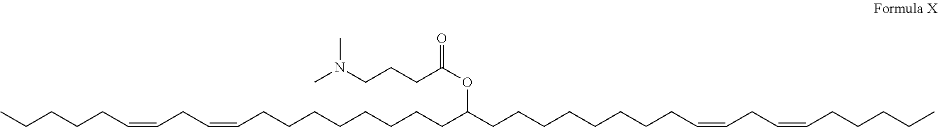 [SMILES: CCCCC/C=C\C/C=C\CCCCCCCCC(CCCCCCCC/C=C\C/C=C\CCCCC)OC(=O)CCCN(C)C]

In one embodiment, the cleavable lipid is not the lipid ATX-002. The lipid ATX-002 is described in WO2015/074085, the content of which is incorporated herein by reference in its entirety. In one embodiment, the cleavable lipid is not (13Z.16Z)-/V,/V-dimethyl-3-nonyldocosa-13,16-dien-1-amine (Compound 32). Compound 32 is described in WO2012/040184, the contents of which is incorporated herein by reference in its entirety. In one embodiment, the cleavable lipid is not Compound 6 or Compound 22. Compounds 6 and 22 are described in WO2015/199952, the content of which is incorporated herein by reference in its entirety.

In one embodiment, the lipid particles (e.g., lipid nanoparticles) can further comprise a non-cationic lipid. The non-cationic lipid can serve to increase fusogenicity and also increase stability of the LNP during formation. Non-cationic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is to be understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Other examples of non-cationic lipids suitable for use in the lipid particles (e.g., lipid nanoparticles) include nonphosphorous lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In one embodiment, the non-cationic lipid is a phospholipid. In one embodiment, the non-cationic lipid is selected from the group consisting of DSPC, DPPC, DMPC, DOPC, POPC, DOPE, and SM. In some embodiments, the non-cationic lipid is DSPC. In other embodiments, the non-cationic lipid is DOPC. In other embodiments, the non-cationic lipid is DOPE.

In some embodiments, the non-cationic lipid can comprise 0-20% (mol) of the total lipid present in the lipid nanoparticle. In some embodiments, the non-cationic lipid content is 0.5-15% (mol) of the total lipid present in the lipid particle (e.g., lipid nanoparticle). In some embodiments, the non-cationic lipid content is 5-12% (mol) of the total lipid present in the lipid particle (e.g., lipid nanoparticle). In some embodiments, the non-cationic lipid content is 5-10% (mol) of the total lipid present in the lipid particle (e.g., lipid nanoparticle). In one embodiment, the non-cationic lipid content is about 6% (mol) of the total lipid present in the lipid particle (e.g., lipid nanoparticle). In one embodiment, the non-cationic lipid content is about 7.0% (mol) of the total lipid present in the lipid particle (e.g., lipid nanoparticle). In one embodiment, the non-cationic lipid content is about 7.5% (mol) of the total lipid present in the lipid particle (e.g., lipid nanoparticle). In one embodiment, the non-cationic lipid content is about 8.0% (mol) of the total lipid present in the lipid particle (e.g., lipid nanoparticle). In one embodiment, the non-cationic lipid content is about 9.0% (mol) of the total lipid present in the lipid particle (e.g., lipid nanoparticle). In some embodiments, the non-cationic lipid content is about 10% (mol) of the total lipid present in the lipid particle (e.g., lipid nanoparticle). In one embodiment, the non-cationic lipid content is about 11% (mol) of the total lipid present in the lipid particle (e.g., lipid nanoparticle).

Exemplary non-cationic lipids are described in PCT Publication WO2017/099823 and US patent publication US2018/0028664, the contents of both of which are incorporated herein by reference in their entirety.

Non-limiting examples of cationic lipids include SS-cleavable and pH-activated lipid-like material-OP (ss-OP; Formula I), SS-cleavable and pH-activated lipid-like material-M (SS-M; Formula V), SS-cleavable and pH-activated lipid-like material-E (SS-E; Formula VI), SS-cleavable and pH-activated lipid-like material-EC (SS-EC; Formula VII), SS-cleavable and pH-activated lipid-like material-LC (SS-LC; Formula VIII), SS-cleavable and pH-activated lipid-like material-OC (SS-OC; Formula IX), polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3b-[N-(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Nucleic acids (e.g., ceDNA or CELiD) can also be complexed with, e.g., poly (L-lysine) or avidin and lipids can, or cannot, be included in this mixture, e.g., steryl-poly (L-lysine).

In one embodiment, the cationic lipid is ss-OP of Formula I. In another embodiment, the cationic lipid SS-PAZ of Formula II.

In one embodiment, a ceDNA vector as disclosed herein is delivered using a cationic lipid described in U.S. Pat. No. 8,158,601, or a polyamine compound or lipid as described in U.S. Pat. No. 8,034,376.

In one embodiment, the lipid particles (e.g., lipid nanoparticles) can further comprise a component, such as a sterol, to provide membrane integrity and stability of the lipid particle. In one embodiment, an exemplary sterol that can be used in the lipid particle is cholesterol, or a derivative thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In some embodiments, the cholesterol derivative is a polar analogue such as cholesteryl-(4′-hydroxy)-butyl ether. In some embodiments, cholesterol derivative is cholestryl hemisuccinate (CHEMS).

Exemplary cholesterol derivatives are described in PCT publication WO2009/127060 and US patent publication US2010/0130588, contents of both of which are incorporated herein by reference in their entirety.

In one embodiment, the component providing membrane integrity, such as a sterol, can comprise 0-50% (mol) of the total lipid present in the lipid particle (e.g., lipid nanoparticle). In some embodiments, such a component is 20-50% (mol) of the total lipid content of the lipid particle (e.g., lipid nanoparticle). In some embodiments, such a component is 30-40% (mol) of the total lipid content of the lipid particle (e.g., lipid nanoparticle). In some embodiments, such a component is 35-45% (mol) of the total lipid content of the lipid particle (e.g., lipid nanoparticle). In some embodiments, such a component is 38-42% (mol) of the total lipid content of the lipid particle (e.g., lipid nanoparticle).

In one embodiment, the lipid particle (e.g., lipid nanoparticle) can further comprise a polyethylene glycol (PEG) or a conjugated lipid molecule. Generally, these are used to inhibit aggregation of lipid particle (e.g., lipid nanoparticle) and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid. In some other embodiments, the conjugated lipid molecule is a PEG-lipid conjugate, for example, a PEG₂₀₀₀-DMG (dimyristoylglycerol).

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In one embodiment, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In one embodiment, the PEG-lipid can be selected from the group consisting of PEG-DMG, 1,2-dimyristoyl-sn-glycero-3-phosphoethanolamine-N-[methoxy(polyethylene glycol)-2000].

In one embodiment, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the PCT patent application publications WO 1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and US patents U.S. Pat. Nos. 5,885,613, 6,287,591, 6,320,017, and 6,586,559, the contents of all of which are incorporated herein by reference in their entireties.

In some embodiments, the PEG or the conjugated lipid can comprise 0-20% (mol) of the total lipid present in the lipid nanoparticle. In some embodiments, PEG or the conjugated lipid content is 2-10% (mol) of the total lipid present in the lipid particle (e.g., lipid nanoparticle). In some embodiments, PEG or the conjugated lipid content is 2-5% (mol) of the total lipid present in the lipid particle (e.g., lipid nanoparticle). In some embodiments, PEG or the conjugated lipid content is 2-3% (mol) of the total lipid present in the lipid particle (e.g., lipid nanoparticle). In one embodiment, PEG or the conjugated lipid content is about 2.5% (mol) of the total lipid present in the lipid particle (e.g., lipid nanoparticle). In some embodiments, PEG or the conjugated lipid content is about 3% (mol) of the total lipid present in the lipid particle (e.g., lipid nanoparticle).

It is understood that molar ratios of the cationic lipid, e.g., ionizable cationic lipid, with the non-cationic-lipid, sterol, and PEG/conjugated lipid can be varied as needed. For example, the lipid particle (e.g., lipid nanoparticle) can comprise 30-70% cationic lipid by mole or by total weight of the composition, 0-60% cholesterol by mole or by total weight of the composition, 0-30% non-cationic-lipid by mole or by total weight of the composition and 1-10% PEG or the conjugated lipid by mole or by total weight of the composition. In one embodiment, the composition comprises 40-60% cationic lipid by mole or by total weight of the composition, 30-50% cholesterol by mole or by total weight of the composition, 5-15% non-cationic-lipid by mole or by total weight of the composition and 1-5% PEG or the conjugated lipid by mole or by total weight of the composition. In one embodiment, the composition is 40-60% cationic lipid by mole or by total weight of the composition, 30-40% cholesterol by mole or by total weight of the composition, and 5-10% non-cationic lipid, by mole or by total weight of the composition and 1-5% PEG or the conjugated lipid by mole or by total weight of the composition. The composition may contain 60-70% cationic lipid by mole or by total weight of the composition, 25-35% cholesterol by mole or by total weight of the composition, 5-10% non-cationic-lipid by mole or by total weight of the composition and 0-5% PEG or the conjugated lipid by mole or by total weight of the composition. The composition may also contain up to 45-55% cationic lipid by mole or by total weight of the composition, 35-45% cholesterol by mole or by total weight of the composition, 2 to 15% non-cationic lipid by mole or by total weight of the composition, and 1-5% PEG or the conjugated lipid by mole or by total weight of the composition. The formulation may also be a lipid nanoparticle formulation, for example comprising 8-30% cationic lipid by mole or by total weight of the composition, 5-15% non-cationic lipid by mole or by total weight of the composition, and 0-40% cholesterol by mole or by total weight of the composition; 4-25% cationic lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% cationic lipid by mole or by total weight of the composition, 2-30% non-cationic lipid by mole or by total weight of the composition, 1 to 15% cholesterol by mole or by total weight of the composition, 2 to 35% PEG or the conjugate lipid by mole or by total weight of the composition, and 1-20% cholesterol by mole or by total weight of the composition; or even up to 90% cationic lipid by mole or by total weight of the composition and 2-10% non-cationic lipids by mole or by total weight of the composition, or even 100% cationic lipid by mole or by total weight of the composition. In some embodiments, the lipid particle formulation comprises cationic lipid, non-cationic phospholipid, cholesterol and a PEG-ylated lipid (conjugated lipid) in a molar ratio of about 50:10:38.5:1.5.

In one embodiment, the lipid particle (e.g., lipid nanoparticle) formulation comprises cationic lipid, non-cationic phospholipid, cholesterol and a PEG-ylated lipid (conjugated lipid) in a molar ratio of about 50:7:40:3.

In one embodiment, the lipid particle (e.g., lipid nanoparticle) comprises cationic lipid, non-cationic lipid (e.g. phospholipid), a sterol (e.g., cholesterol) and a PEG-ylated lipid (conjugated lipid), where the molar ratio of lipids ranges from 20 to 70 mole percent for the cationic lipid, with a target of 30-60, the mole percent of non-cationic lipid ranges from 0 to 30, with a target of 0 to 15, the mole percent of sterol ranges from 20 to 70, with a target of 30 to 50, and the mole percent of PEG-ylated lipid (conjugated lipid) ranges from 1 to 6, with a target of 2 to 5.

Lipid nanoparticles (LNPs) comprising ceDNA are disclosed in International Application PCT/US2018/050042, filed on Sep. 7, 2018, which is incorporated herein in its entirety and envisioned for use in the methods and compositions as disclosed herein.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al., Nature Biotechnology 28, 172-176 (20 1 0), both of which are incorporated by reference in their entireties). In one embodiment, the pKa of each cationic lipid is determined in lipid nanoparticles using an assay based on fluorescence of 2-(p-toluidino)-6-napthalene sulfonic acid (TNS). Lipid nanoparticles comprising of cationic lipid/DSPC/cholesterol/PEG-lipid (50/10/38.5/1.5 mol %) in PBS at a concentration of 0.4 mM total lipid can be prepared using the in-line process as described herein and elsewhere. TNS can be prepared as a 100 mM stock solution in distilled water. Vesicles can be diluted to 24 mM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 mM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

In one embodiment, relative activity can be determined by measuring luciferase expression in the liver 4 hours following administration via tail vein injection. The activity is compared at a dose of 0.3 and 1.0 mg ceDNA/kg and expressed as ng luciferase/g liver measured 4 hours after administration.

Without limitations, a lipid particle (e.g., lipid nanoparticle) of the present disclosure includes a lipid formulation that can be used to deliver a capsid-free, non-viral DNA vector to a target site of interest (e.g., cell, tissue, organ, and the like). Generally, the lipid particle (e.g., lipid nanoparticle) comprises capsid-free, non-viral DNA vector and a cationic lipid or a salt thereof.

In one embodiment, the lipid particle (e.g., lipid nanoparticle) comprises a cationic lipid/non-cationic-lipid/sterol/conjugated lipid at a molar ratio of 50:10:38.5:1.5. In another embodiment, the lipid particle (e.g., lipid nanoparticle) comprises a cationic lipid/non-cationic-lipid/sterol/conjugated lipid at a molar ratio of 50:10:37.5:2.5. In one embodiment, the disclosure provides for a lipid particle formulation comprising phospholipids, lecithin, phosphatidylcholine and phosphatidylethanolamine.

### III. Therapeutic Nucleic Acids

Nucleic acids are large, highly charged, rapidly degraded and cleared from the body, and offer generally poor pharmacological properties because they are recognized as a foreign matter to the body and become a target of an innate immune response. Hence, certain therapeutic nucleic acids (“TNAs”) (e.g., antisense oligonucleotide or viral vectors) can often trigger immune responses in vivo. The present disclosure provides pharmaceutical compositions and methods that may ameliorate, reduce or eliminate such immune responses and enhance efficacy of therapeutic nucleic acids by increasing expression levels through maximizing the durability of the therapeutic nucleic acid in a reduced immune-responsive state in a subject recipient. This may also minimize any potential adverse events that may lead to an organ damage or other toxicity in the course of gene therapy.

Illustrative therapeutic nucleic acids of the present disclosure can include, but are not limited to, minigenes, plasmids, minicircles, small interfering RNA (siRNA), microRNA (miRNA), antisense oligonucleotides (ASO), ribozymes, closed ended double stranded DNA (e.g., ceDNA, CELiD, linear covalently closed DNA (“ministring”), Doggybone™, protelomere closed ended DNA, or dumbbell linear DNA), dicer-substrate dsRNA, small hairpin RNA (shRNA), asymmetrical interfering RNA (aiRNA), microRNA (miRNA), mRNA, tRNA, rRNA, and DNA viral vectors, viral RNA vector, and any combination thereof.

siRNA or miRNA that can downregulate the intracellular levels of specific proteins through a process called RNA interference (RNAi) are also contemplated by the present invention to be nucleic acid therapeutics. After siRNA or miRNA is introduced into the cytoplasm of a host cell, these double-stranded RNA constructs can bind to a protein called RISC. The sense strand of the siRNA or miRNA is removed by the RISC complex. The RISC complex, when combined with the complementary mRNA, cleaves the mRNA and release the cut strands. RNAi is by inducing specific destruction of mRNA that results in downregulation of a corresponding protein.

Antisense oligonucleotides (ASO) and ribozymes that inhibit mRNA translation into protein can be nucleic acid therapeutics. For antisense constructs, these single stranded deoxy nucleic acids have a complementary sequence to the sequence of the target protein mRNA, and Watson—capable of binding to the mRNA by Crick base pairing. This binding prevents translation of a target mRNA, and/or triggers RNaseH degradation of the mRNA transcript. As a result, the antisense oligonucleotide has increased specificity of action (i.e., down-regulation of a specific disease-related protein).

In any of the methods provided herein, the therapeutic nucleic acid can be a therapeutic RNA. Said therapeutic RNA can be an inhibitor of mRNA translation, agent of RNA interference (RNAi), catalytically active RNA molecule (ribozyme), transfer RNA (tRNA) or an RNA that binds an mRNA transcript (ASO), protein or other molecular ligand (aptamer). In any of the methods provided herein, the agent of RNAi can be a double-stranded RNA, single-stranded RNA, micro RNA, short interfering RNA, short hairpin RNA, or a triplex-forming oligonucleotide.

According to some embodiments, the therapeutic nucleic acid is a closed ended double stranded DNA, e.g., a ceDNA. According to some embodiments, the expression and/or production of a therapeutic protein in a cell is from a non-viral DNA vector, e.g., a ceDNA vector. A distinct advantage of ceDNA vectors for expression of a therapeutic protein over traditional AAV vectors, and even lentiviral vectors, is that there is no size constraint for the heterologous nucleic acid sequences encoding a desired protein. Thus, even a large therapeutic protein can be expressed from a single ceDNA vector. Thus, ceDNA vectors can be used to express a therapeutic protein in a subject in need thereof.

In general, a ceDNA vector for expression of a therapeutic protein as disclosed herein, comprises in the 5′ to 3′ direction: a first adeno-associated virus (AAV) inverted terminal repeat (ITR), a nucleotide sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

### IV. Closed-Ended DNA (ceDNA) Vectors

Aspects of the present disclosure generally provide lipid particles (e.g., lipid nanoparticles) comprising a capsid free, non-viral closed-ended DNA vector and a lipid.

Embodiments of the disclosure are based on methods and compositions comprising closed-ended linear duplexed (ceDNA) vectors that can express a transgene (e.g. a therapeutic nucleic acid). The ceDNA vectors as described herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

There are many structural features of ceDNA vectors that differ from plasmid-based expression vectors. ceDNA vectors may possess one or more of the following features: the lack of original (i.e. not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, of the eukaryotic origin (i.e., they are produced in eukaryotic cells), and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a nonlimiting example in a promoter or enhancer region. Another important feature distinguishing ceDNA vectors from plasmid expression vectors is that ceDNA vectors are single-strand linear DNA having closed ends, while plasmids are always double-stranded DNA.

There are several advantages of using a ceDNA vector as described herein over plasmid-based expression vectors, such advantages include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ceDNA vectors do not; 3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′(SEQ ID NO: 1) for AAV2) and a terminal resolution site (TRS; 5′-AGTTGG-3′ for AAV2) plus a variable palindromic sequence allowing for hairpin formation; and 4) ceDNA vectors do not have the over representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

ceDNA vectors preferably have a linear and continuous structure rather than a non-continuous structure. The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In some embodiments, ceDNA vectors can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects, and also in view of their DNA methylation which is of prokaryotic type for ceDNA-plasmids and of eukaryotic type for the ceDNA vector.

Provided herein are non-viral, capsid-free ceDNA molecules with covalently-closed ends (ceDNA). These non-viral capsid free ceDNA molecules can be produced in permissive host cells from an expression construct (e.g., a ceDNA-plasmid, a ceDNA-bacmid, a ceDNA-baculovirus, or an integrated cell-line) containing a heterologous gene (e.g., a transgene, in particular a therapeutic transgene) positioned between two different inverted terminal repeat (ITR) sequences, where the ITRs are different with respect to each other. In some embodiments, one of the ITRs is modified by deletion, insertion, and/or substitution as compared to a wild-type ITR sequence (e.g. AAV ITR); and at least one of the ITRs comprises a functional terminal resolution site (trs) and a Rep binding site. The ceDNA vector is preferably duplex, e.g., self-complementary, over at least a portion of the molecule, such as the expression cassette (e.g. ceDNA is not a double stranded circular molecule). The ceDNA vector has covalently closed ends, and thus is resistant to exonuclease digestion (e.g. exonuclease I or exonuclease III), e.g. for over an hour at 37° C.

In one aspect, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR. In one embodiment, the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In one embodiment, the first ITR and the second ITR are both modified but are different sequences, or have different modifications, or are not identical modified ITRs, and have different 3D spatial configurations. Stated differently, a ceDNA vector with asymmetric ITRs have ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other.

In one embodiment, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild type ITRs. A mod-ITR pair can have the same sequence which has one or more modifications from wild-type ITR and are reverse complements (inverted) of each other. In one embodiment, a modified ITR pair are substantially symmetrical as defined herein, that is, the modified ITR pair can have a different sequence but have corresponding or the same symmetrical three-dimensional shape. In some embodiments, the symmetrical ITRs, or substantially symmetrical ITRs can be are wild type (WT-ITRs) as described herein. That is, both ITRs have a wild type sequence, but do not necessarily have to be WT-ITRs from the same AAV serotype. In one embodiment, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

The wild-type or mutated or otherwise modified ITR sequences provided herein represent DNA sequences included in the expression construct (e.g., ceDNA-plasmid, ceDNA Bacmid, ceDNA-baculovirus) for production of the ceDNA vector. Thus, ITR sequences actually contained in the ceDNA vector produced from the ceDNA-plasmid or other expression construct may or may not be identical to the ITR sequences provided herein as a result of naturally occurring changes taking place during the production process (e.g., replication error).

In one embodiment, a ceDNA vector described herein comprising the expression cassette with a transgene which is a therapeutic nucleic acid sequence, can be operatively linked to one or more regulatory sequence(s) that allows or controls expression of the transgene. In one embodiment, the polynucleotide comprises a first ITR sequence and a second ITR sequence, wherein the nucleotide sequence of interest is flanked by the first and second ITR sequences, and the first and second ITR sequences are asymmetrical relative to each other, or symmetrical relative to each other.

In one embodiment, an expression cassette is located between two ITRs comprised in the following order with one or more of: a promoter operably linked to a transgene, a posttranscriptional regulatory element, and a polyadenylation and termination signal. In one embodiment, the promoter is regulatable—inducible or repressible. The promoter can be any sequence that facilitates the transcription of the transgene. In one embodiment the promoter is a CAG promoter, or variation thereof. The posttranscriptional regulatory element is a sequence that modulates expression of the transgene, as a non-limiting example, any sequence that creates a tertiary structure that enhances expression of the transgene which is a therapeutic nucleic acid sequence.

In one embodiment, the posttranscriptional regulatory element comprises WPRE. In one embodiment, the polyadenylation and termination signal comprise BGHpolyA. Any cis regulatory element known in the art, or combination thereof, can be additionally used e.g., SV40 late polyA signal upstream enhancer sequence (UES), or other posttranscriptional processing elements including, but not limited to, the thymidine kinase gene of herpes simplex virus, or hepatitis B virus (HBV). In one embodiment, the expression cassette length in the 5′ to 3′ direction is greater than the maximum length known to be encapsidated in an AAV virion. In one embodiment, the length is greater than 4.6 kb, or greater than 5 kb, or greater than 6 kb, or greater than 7 kb. Various expression cassettes are exemplified herein.

In one embodiment, the expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene which is a therapeutic nucleic acid sequence in the range of 500 to 50,000 nucleotides in length. In one embodiment, the expression cassette can comprise a transgene which is a therapeutic nucleic acid sequence in the range of 500 to 75,000 nucleotides in length. In one embodiment, the expression cassette can comprise a transgene which is a therapeutic nucleic acid sequence in the range of 500 to 10,000 nucleotides in length. In one embodiment, the expression cassette can comprise a transgene which is a therapeutic nucleic acid sequence in the range of 1000 to 10,000 nucleotides in length. In one embodiment, the expression cassette can comprise a transgene which is a therapeutic nucleic acid sequence in the range of 500 to 5,000 nucleotides in length. The ceDNA vectors do not have the size limitations of encapsidated AAV vectors, and thus enable delivery of a large-size expression cassette to the host. In one embodiment, the ceDNA vector is devoid of prokaryote-specific methylation.

In one embodiment, the expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene, for example, a regulatory switch, for controlling and regulating the expression of the transgene, and can include if desired, a regulatory switch which is a kill switch to enable controlled cell death of a cell comprising a ceDNA vector.

In one embodiment, ceDNA vectors are capsid-free and can be obtained from a plasmid encoding in this order: a first ITR, expressible transgene cassette and a second ITR, where at least one of the first and/or second ITR sequence is mutated with respect to the corresponding wild type AAV2 ITR sequence.

In one embodiment, the ceDNA vectors disclosed herein are used for therapeutic purposes (e.g., for medical, diagnostic, or veterinary uses) or immunogenic polypeptides.

The expression cassette can comprise any transgene which is a therapeutic nucleic acid sequence. In certain embodiments, the ceDNA vector comprises any gene of interest in the subject, which includes one or more polypeptides, peptides, ribozymes, peptide nucleic acids, siRNAs, RNAis, antisense oligonucleotides, antisense polynucleotides, antibodies, antigen binding fragments, or any combination thereof.

In one embodiment, the ceDNA expression cassette can include, for example, an expressible exogenous sequence (e.g., open reading frame) that encodes a protein that is either absent, inactive, or insufficient activity in the recipient subject or a gene that encodes a protein having a desired biological or a therapeutic effect. In one embodiment, the exogenous sequence such as a donor sequence can encode a gene product that can function to correct the expression of a defective gene or transcript. In one embodiment, the expression cassette can also encode corrective DNA strands, encode polypeptides, sense or antisense oligonucleotides, or RNAs (coding or non-coding; e.g., siRNAs, shRNAs, micro-RNAs, and their antisense counterparts (e.g., antagoMiR)). In one embodiment, expression cassettes can include an exogenous sequence that encodes a reporter protein to be used for experimental or diagnostic purposes, such as b-lactamase, b-galactosidase (LacZ), alkaline phosphatase, thymidine kinase, green fluorescent protein (GFP), chloramphenicol acetyltransferase (CAT), luciferase, and others well known in the art.

Accordingly, the expression cassette can include any gene that encodes a protein, polypeptide or RNA that is either reduced or absent due to a mutation or which conveys a therapeutic benefit when overexpressed is considered to be within the scope of the disclosure. The ceDNA vector may comprise a template or donor nucleotide sequence used as a correcting DNA strand to be inserted after a double-strand break (or nick) provided by a nuclease. The ceDNA vector may include a template nucleotide sequence used as a correcting DNA strand to be inserted after a double-strand break (or nick) provided by a guided RNA nuclease, meganuclease, or zinc finger nuclease.

Preferably, non-inserted bacterial DNA is not present and preferably no bacterial DNA is present in the ceDNA compositions provided herein. In some instances, the protein can change a codon without a nick.

In one embodiment, sequences provided in the expression cassette, expression construct, or donor sequence of a ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid.

Typically, codon optimization does not alter the amino acid sequence of the original translated protein. Optimized codons can be determined using e.g., Aptagen's Gene Forge® codon optimization and custom gene synthesis platform (Aptagen, Inc., 2190 Fox Mill Rd. Suite 300, Herndon, Va. 20171) or another publicly available database.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage (Nakamura, Y., et al. “Codon usage tabulated from the international DNA sequence databases: status for the year 2000” Nucl. Acids Res. 28:292 (2000)).

There are many structural features of ceDNA vectors that differ from plasmid-based expression vectors. ceDNA vectors may possess one or more of the following features: the lack of original (i.e. not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, of the eukaryotic origin (i.e., they are produced in eukaryotic cells), and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a nonlimiting example in a promoter or enhancer region. Another important feature distinguishing ceDNA vectors from plasmid expression vectors is that ceDNA vectors are single-strand linear DNA having closed ends, while plasmids are always double-stranded DNA.

In one embodiment, ceDNA vectors produced by the methods provided herein preferably have a linear and continuous structure rather than a non-continuous structure. The linear and continuous structure is believed to be more stable from attack by cellular endonucleases, as well as less likely to be recombined and cause mutagenesis. Accordingly, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects, and also in view of their DNA methylation which is of prokaryotic type for ceDNA-plasmids and of eukaryotic type for the ceDNA vector.

### Example 1

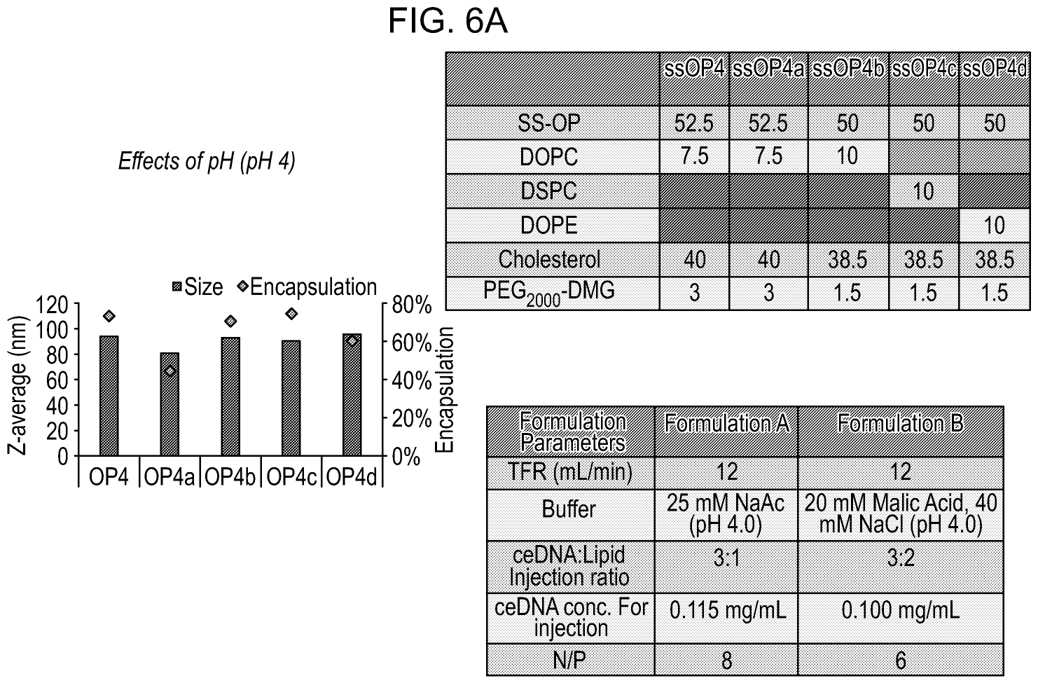

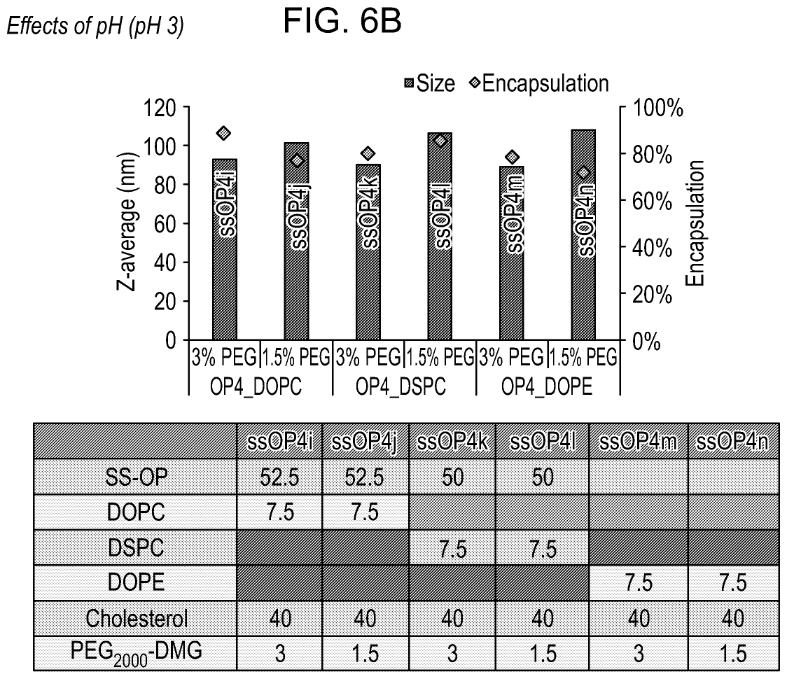

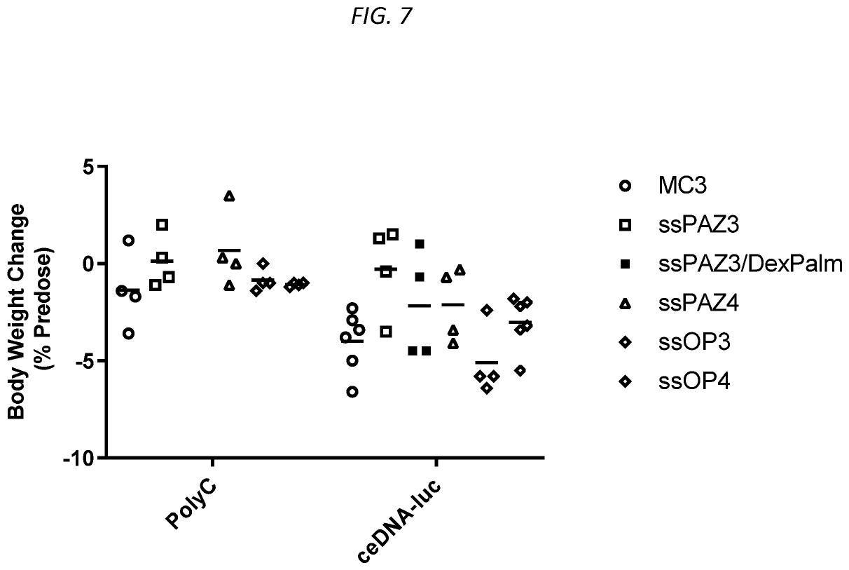

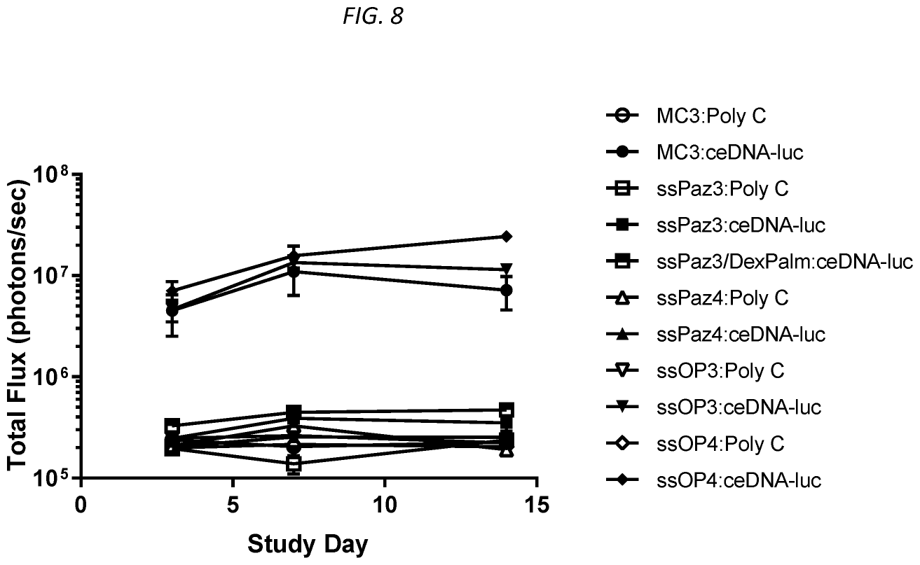

According to some embodiments, synthetic ceDNA is produced via excision from a double-stranded DNA molecule. Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIG. 6 in International patent application PCT/US2018/064242, filed Dec. 6, 2018).

In some embodiments, a construct to make a ceDNA vector (e.g., a synthetic AAV vector) comprises additional components to regulate expression of the transgene, for example, regulatory switches, to regulate the expression of the transgene, or a kill switch, which can kill a cell comprising the vector.

A molecular regulatory switch is one which generates a measurable change in state in response to a signal. Such regulatory switches can be usefully combined with the ceDNA vectors described herein to control the output of expression of the transgene. In some embodiments, the ceDNA vector comprises a regulatory switch that serves to fine tune expression of the transgene. For example, it can serve as a biocontainment function of the ceDNA vector. In some embodiments, the switch is an “ON/OFF” switch that is designed to start or stop (i.e., shut down) expression of the gene of interest in the ceDNA vector in a controllable and regulatable fashion. In some embodiments, the switch can include a “kill switch” that can instruct the cell comprising the synthetic ceDNA vector to undergo cell programmed death once the switch is activated. Exemplary regulatory switches encompassed for use in a ceDNA vector can be used to regulate the expression of a transgene, and are more fully discussed in International application PCT/US18/49996, which is incorporated herein in its entirety by reference and described herein.

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122, incorporated by reference in its entirety herein, shows an exemplary method of ligating a 5′ ITR oligonucleotide and a 3′ ITR oligonucleotide to a double stranded polynucleotide comprising an expression cassette.

An exemplary method of producing a ceDNA vector using a synthetic method is provided in Example 4 of PCT/US19/14122, incorporated by reference in its entirety herein, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

In yet another aspect, the invention provides for host cell lines that have stably integrated the DNA vector polynucleotide expression template (ceDNA template) described herein, into their own genome for use in production of the non-viral DNA vector. Methods for producing such cell lines are described in Lee, L. et al. (2013) Plos One 8(8): e69879, which is herein incorporated by reference in its entirety. Preferably, the Rep protein (e.g. as described in Example 1) is added to host cells at an MOI of 3. In one embodiment, the host cell line is an invertebrate cell line, preferably insect Sf9 cells. When the host cell line is a mammalian cell line, preferably 293 cells the cell lines can have polynucleotide vector template stably integrated, and a second vector, such as herpes virus can be used to introduce Rep protein into cells, allowing for the excision and amplification of ceDNA in the presence of Rep.

Any promoter can be operably linked to the heterologous nucleic acid (e.g. reporter nucleic acid or therapeutic transgene) of the vector polynucleotide. The expression cassette can contain a synthetic regulatory element, such as CAG promoter. The CAG promoter comprises (i) the cytomegalovirus (CMV) early enhancer element, (ii) the promoter, the first exon and the first intron of the chicken beta actin gene, and (ii) the splice acceptor of the rabbit beta globin gene. Alternatively, expression cassette can contain an Alpha-1-antitrypsin (AAT) promoter, a liver specific (LP1) promoter, or Human elongation factor-1 alpha (EF1-α) promoter. In some embodiments, the expression cassette includes one or more constitutive promoters, for example, the retroviral Rous sarcoma virus (RSV) LTR promoter (optionally with the RSV enhancer), cytomegalovirus (CMV) immediate early promoter (optionally with the CMV enhancer). Alternatively, an inducible or repressible promoter, a native promoter for a transgene, a tissue-specific promoter, or various promoters known in the art can be used. Suitable transgenes for gene therapy are well known to those of skill in the art.

The capsid-free ceDNA vectors can also be produced from vector polynucleotide expression constructs that further comprise cis-regulatory elements, or combination of cis regulatory elements, a non-limiting example include a woodchuck hepatitis virus posttranscriptional regulatory element (WPRE) and BGH polyA, or e.g. beta-globin polyA. Other posttranscriptional processing elements include, e.g. the thymidine kinase gene of herpes simplex virus, or hepatitis B virus (HBV). The expression cassettes can include any poly-adenylation sequence known in the art or a variation thereof, such as a naturally occurring isolated from bovine BGHpA or a virus SV40 pA, or synthetic. Some expression cassettes can also include SV40 late polyA signal upstream enhancer (USE) sequence. The, USE can be used in combination with SV40 pA or heterologous poly-A signal.

The time for harvesting and collecting DNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce DNA-vectors) but before the a majority of cells start to die because of the viral toxicity. The DNA-vectors can be isolated using plasmid purification kits such as Qiagen Endo-Free Plasmid kits. Other methods developed for plasmid isolation can be also adapted for DNA-vectors. Generally, any nucleic acid purification methods can be adopted. The DNA vectors can be purified by any means known to those of skill in the art for purification of DNA. In one embodiment, ceDNA vectors are purified as DNA molecules. In another embodiment, the ceDNA vectors are purified as exosomes or microparticles.

In one embodiment, the capsid free non-viral DNA vector comprises or is obtained from a plasmid comprising a polynucleotide template comprising in this order: a first adeno-associated virus (AAV) inverted terminal repeat (ITR), a nucleotide sequence of interest (for example an expression cassette of an exogenous DNA) and a modified AAV ITR, wherein said template nucleic acid molecule is devoid of AAV capsid protein coding. In a further embodiment, the nucleic acid template of the invention is devoid of viral capsid protein coding sequences (i.e. it is devoid of AAV capsid genes but also of capsid genes of other viruses). In addition, in a particular embodiment, the template nucleic acid molecule is also devoid of AAV Rep protein coding sequences. Accordingly, in a preferred embodiment, the nucleic acid molecule of the invention is devoid of both functional AAV cap and AAV rep genes.

In one embodiment, ceDNA can include an ITR structure that is mutated with respect to the wild type AAV2 ITR disclosed herein, but still retains an operable RBE, TRS and RBE′ portion.

### Inverted Terminal Repeats (ITRs)

As described herein In one embodiment, the ceDNA vectors are capsid-free, linear duplex DNA molecules formed from a continuous strand of complementary DNA with covalently-closed ends (linear, continuous and non-encapsidated structure), which comprise a 5′ inverted terminal repeat (ITR) sequence and a 3′ ITR sequence that are different, or asymmetrical with respect to each other. At least one of the ITRs comprises a functional terminal resolution site and a replication protein binding site (RPS) (sometimes referred to as a replicative protein binding site), e.g. a Rep binding site. Generally, the ceDNA vector contains at least one modified AAV inverted terminal repeat sequence (ITR), i.e., a deletion, insertion, and/or substitution with respect to the other ITR, and an expressible transgene.

In one embodiment, at least one of the ITRs is an AAV ITR, e.g. a wild type AAV ITR. In one embodiment, at least one of the ITRs is a modified ITR relative to the other ITR—that is, the ceDNA comprises ITRs that are asymmetric relative to each other. In one embodiment, at least one of the ITRs is a non-functional ITR.

In one embodiment, the ceDNA vector comprises: (1) an expression cassette comprising a cis-regulatory element, a promoter and at least one transgene; or (2) a promoter operably linked to at least one transgene, and (3) two self-complementary sequences, e.g., ITRs, flanking said expression cassette, wherein the ceDNA vector is not associated with a capsid protein. In some embodiments, the ceDNA vector comprises two self-complementary sequences found in an AAV genome, where at least one comprises an operative Rep-binding element (RBE) and a terminal resolution site (trs) of AAV or a functional variant of the RBE, and one or more cis-regulatory elements operatively linked to a transgene. In some embodiments, the ceDNA vector comprises additional components to regulate expression of the transgene, for example, regulatory switches for controlling and regulating the expression of the transgene, and can include a regulatory switch which is a kill switch to enable controlled cell death of a cell comprising a ceDNA vector.

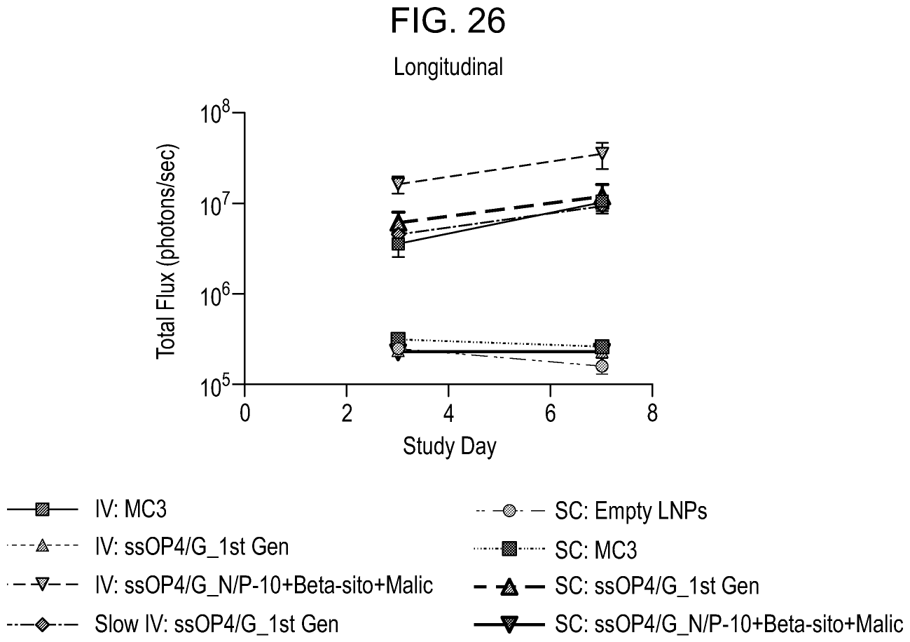

In one embodiment, the two self-complementary sequences can be ITR sequences from any known parvovirus, for example a dependovirus such as AAV (e.g., AAV1-AAV12). Any AAV serotype can be used, including but not limited to a modified AAV2 ITR sequence, that retains a Rep-binding site (RBS) such as 5′-GCGCGCTCGCTCGCTC-3′(SEQ ID NO:1) and a terminal resolution site (trs) in addition to a variable palindromic sequence allowing for hairpin secondary structure formation. In some embodiments, an ITR may be synthetic. In one embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. In some aspects a synthetic ITR may interact preferentially with a wildtype Rep or a Rep of a specific serotype, or in some instances will not be recognized by a wild-type Rep and be recognized only by a mutated Rep. In some embodiments, the ITR is a synthetic ITR sequence that retains a functional Rep-binding site (RBS) such as 5′-GCGCGCTCGCTCGCTC-3′ (SEQ ID NO:1) and a terminal resolution site (TRS) in addition to a variable palindromic sequence allowing for hairpin secondary structure formation. In some examples, a modified ITR sequence retains the sequence of the RBS, trs and the structure and position of a Rep binding element forming the terminal loop portion of one of the ITR hairpin secondary structure from the corresponding sequence of the wild-type AAV2 ITR. Exemplary ITR sequences for use in the ceDNA vectors are disclosed in Tables 2-9, 10A and 10B, SEQ ID NO: 2, 52, 101-449 and 545-547, and the partial ITR sequences shown in FIGS. 26A-26B of PCT application No. PCT/US 18/49996, filed Sep. 7, 2018, the contents of each of which are incorporated by reference in their entireties herein. In some embodiments, a ceDNA vector can comprise an ITR with a modification in the ITR corresponding to any of the modifications in ITR sequences or ITR partial sequences shown in any one or more of Tables 2, 3, 4, 5, 6, 7, 8, 9, 10A and 10B PCT application No. PCT/US 18/49996, filed Sep. 7, 2018.

In one embodiment, the ceDNA vectors can be produced from expression constructs that further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene, for example, regulatory switches as described in PCT application No. PCT/US 18/49996, filed Sep. 7, 2018, to regulate the expression of the transgene or a kill switch, which can kill a cell comprising the ceDNA vector.

In one embodiment, the expression cassettes can also include a post-transcriptional element to increase the expression of a transgene. In one embodiment, Woodchuck Hepatitis Virus (WHP) posttranscriptional regulatory element (WPRE) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences. The expression cassettes can include a poly-adenylation sequence known in the art or a variation thereof, such as a naturally occurring sequence isolated from bovine BGHpA or a virus SV40 pA, or a synthetic sequence. Some expression cassettes can also include SV40 late polyA signal upstream enhancer (USE) sequence. The, USE can be used in combination with SV40 pA or heterologous poly-A signal.

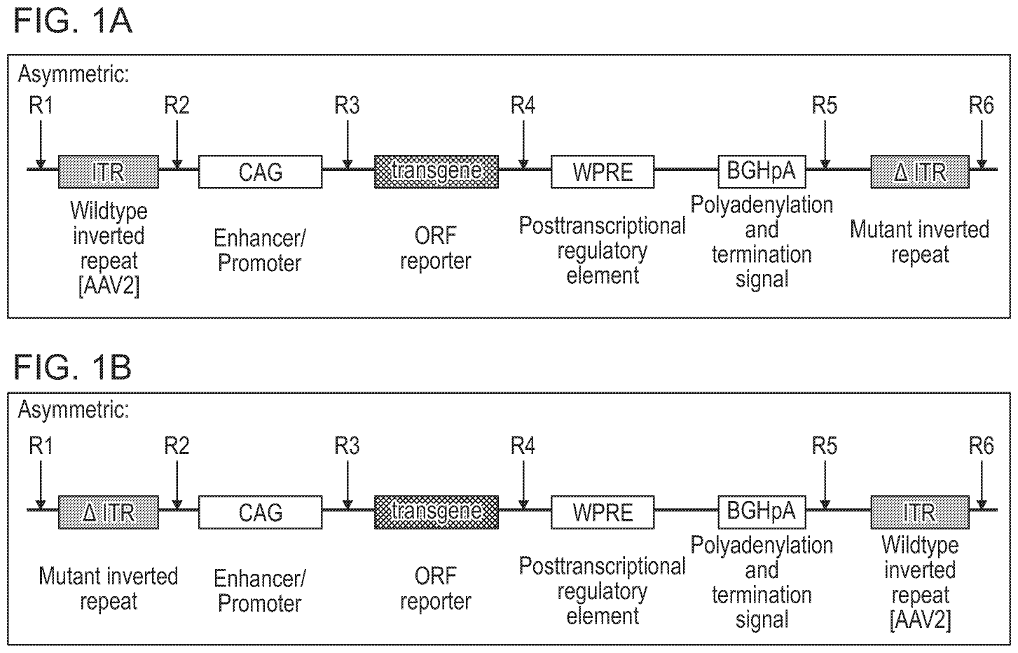

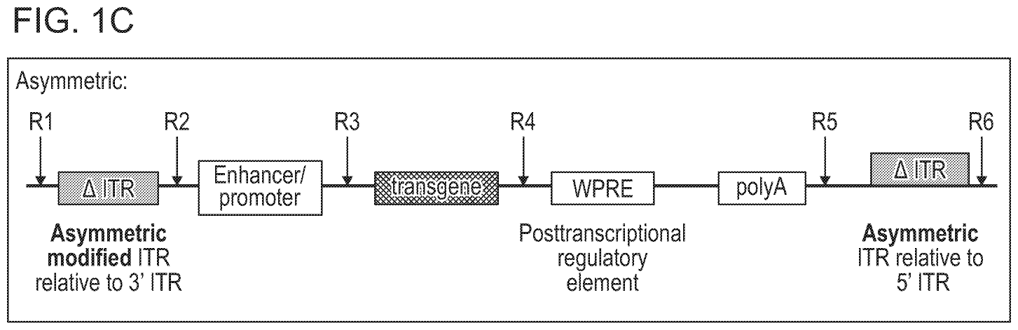

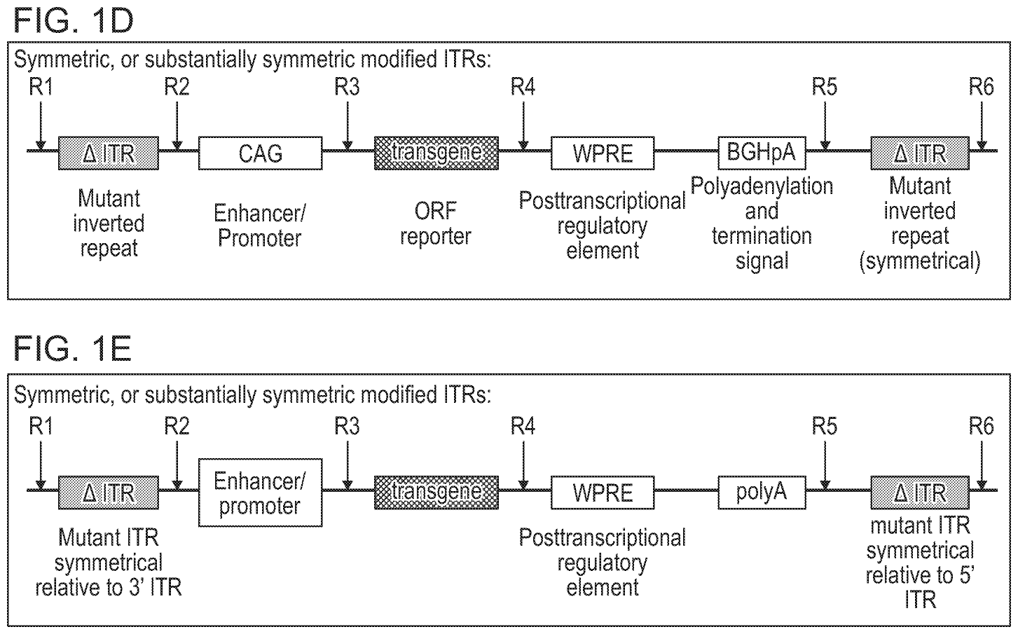

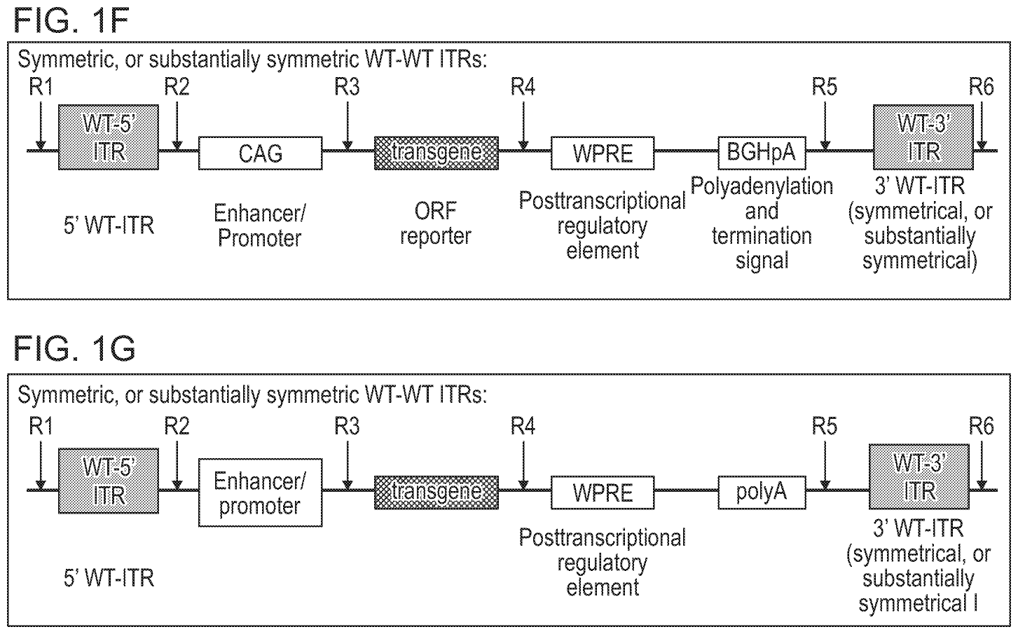

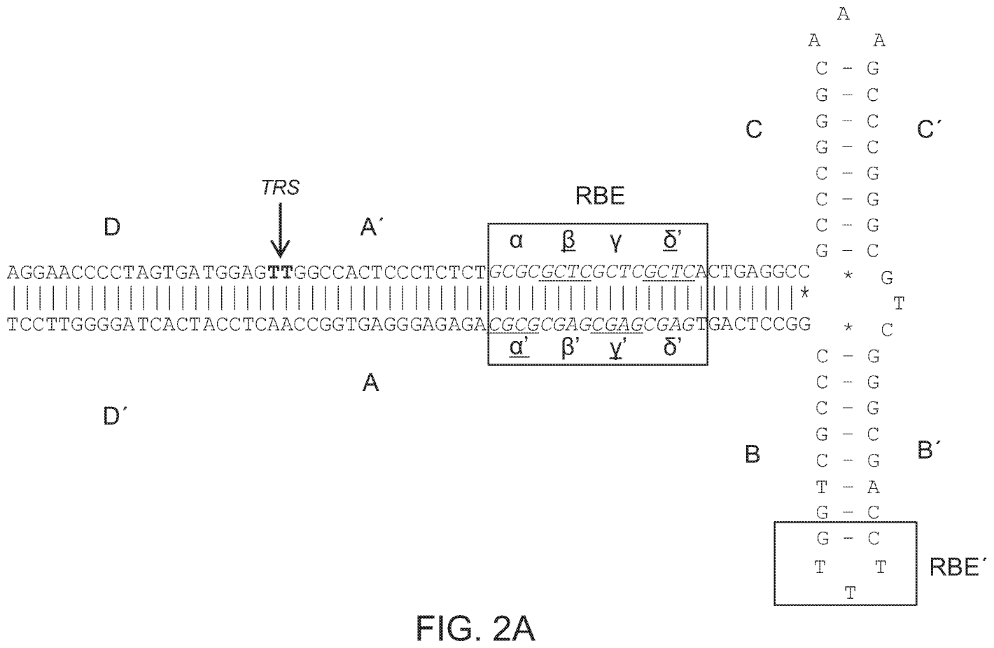

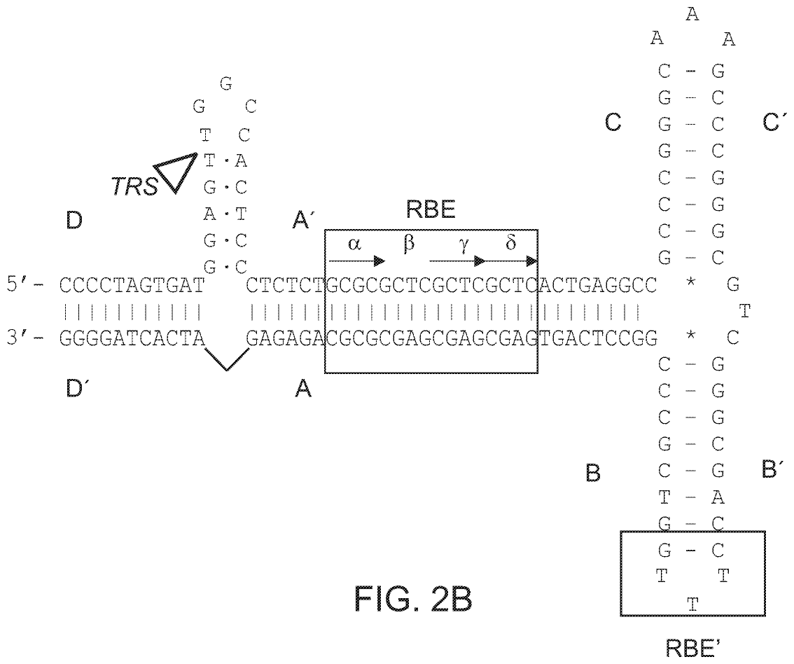

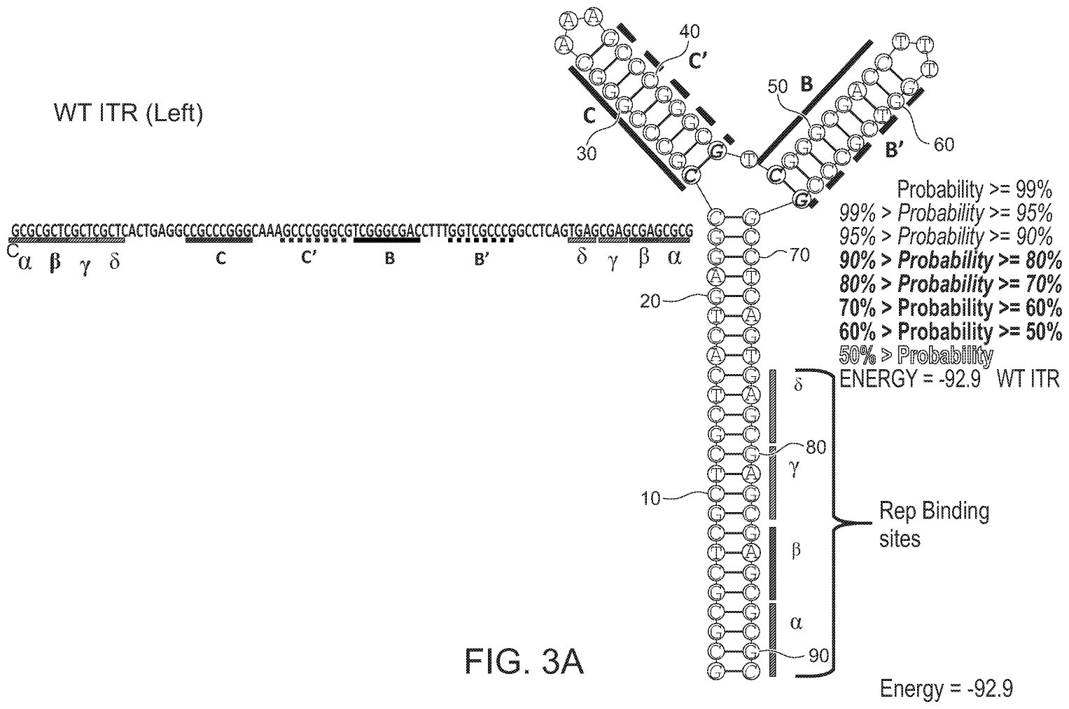

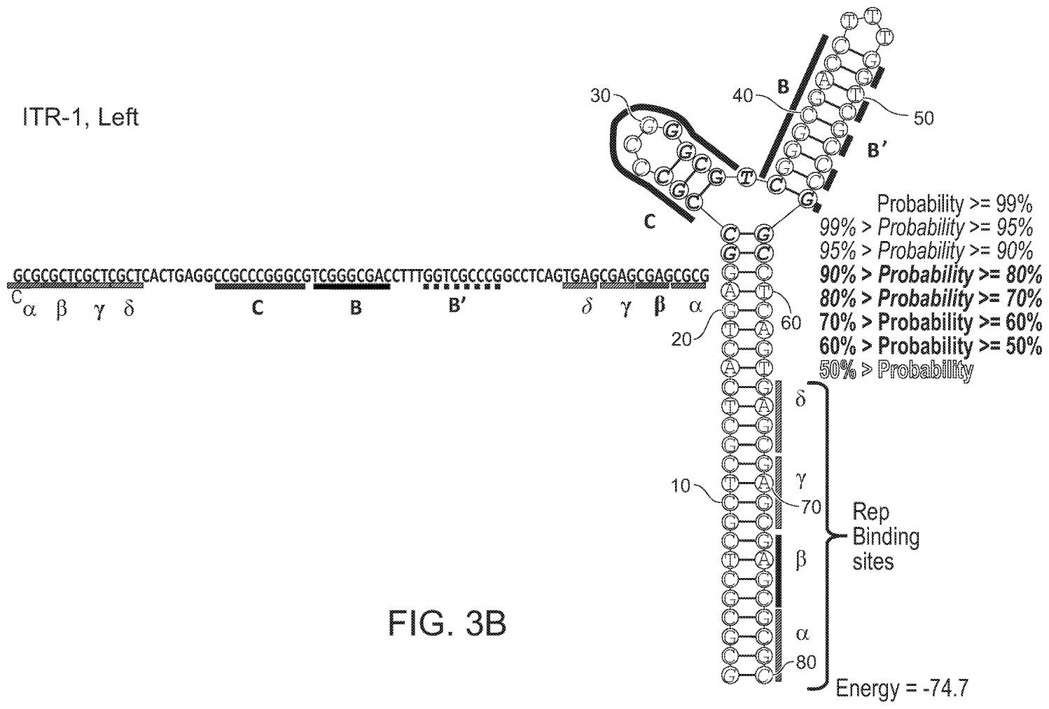

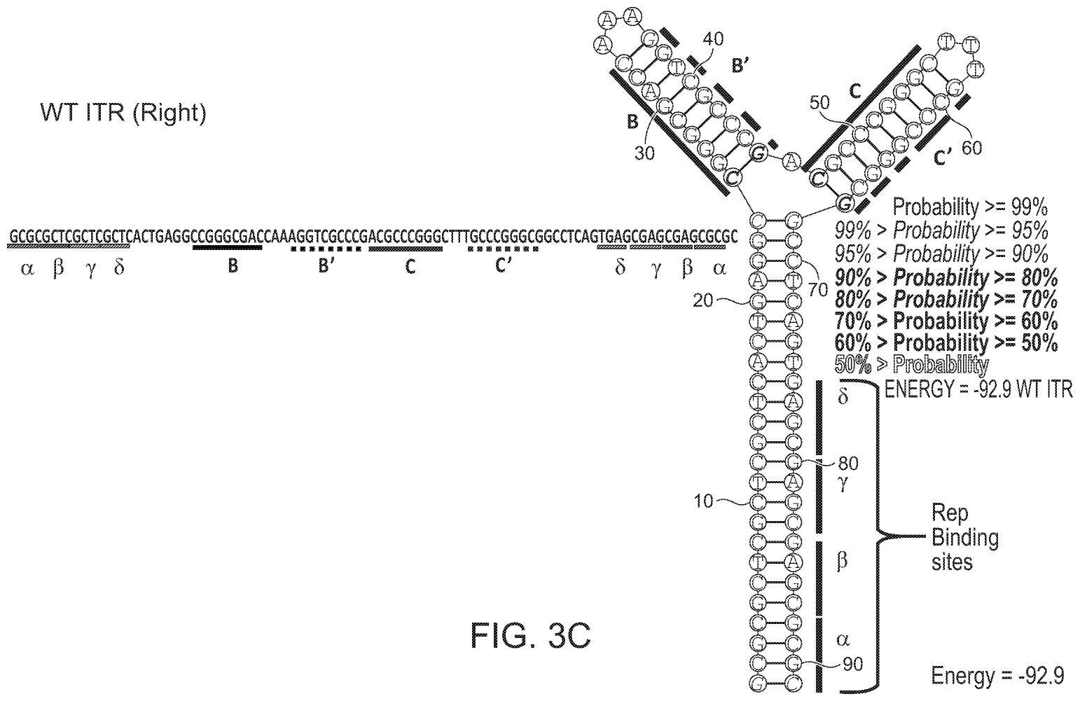

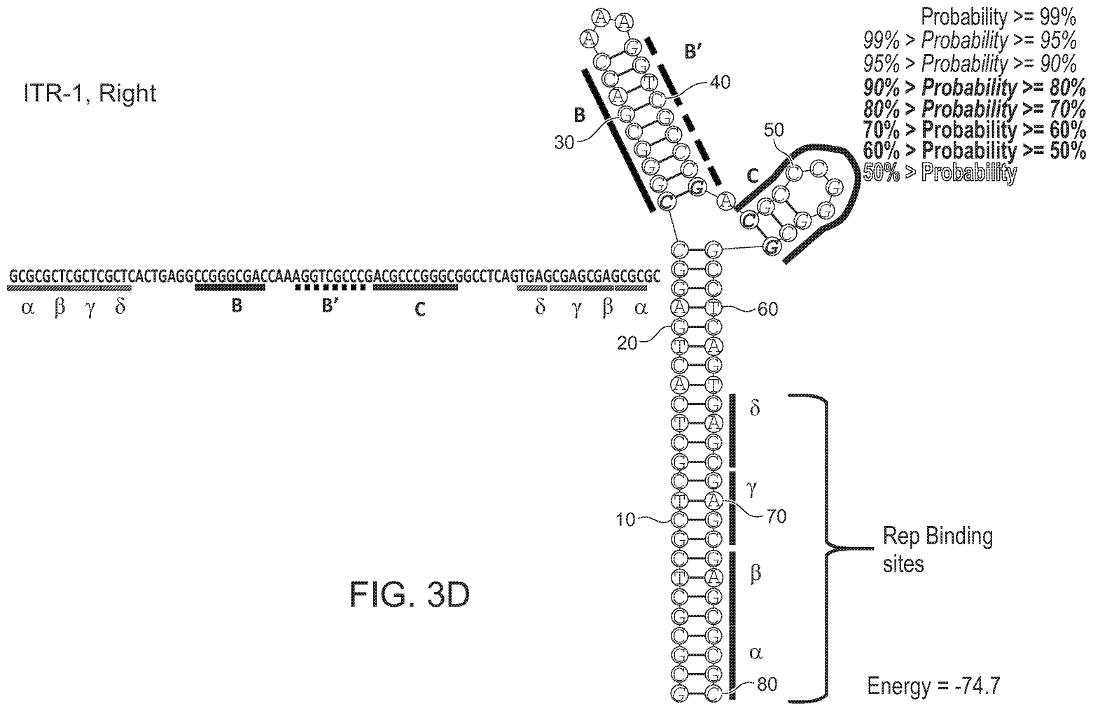

FIGS. 1A-1C of International Application No. PCT/US2018/050042, filed on Sep. 7, 2018 and incorporated by reference in its entirety herein, show schematics of nonlimiting, exemplary ceDNA vectors, or the corresponding sequence of ceDNA plasmids. ceDNA vectors are capsid-free and can be obtained from a plasmid encoding in this order: a first ITR, expressible transgene cassette and a second ITR, where at least one of the first and/or second ITR sequence is mutated with respect to the corresponding wild type AAV2 ITR sequence. The expressible transgene cassette preferably includes one or more of, in this order: an enhancer/promoter, an ORF reporter (transgene), a post-transcription regulatory element (e.g., WPRE), and a polyadenylation and termination signal (e.g., BGH polyA).

### Promoters

Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the S V40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVTE), a rous sarcoma virus (RSV) promoter, a human U6 small nuclear promoter (U6, e.g., (Miyagishi el al., Nature Biotechnology 20, 497-500 (2002)), an enhanced U6 promoter (e.g., Xia et al., Nucleic Acids Res. 2003 Sep. 1; 31(17)), a human H1 promoter (H1), a CAG promoter, a human alpha 1-antitypsin (HAAT) promoter (e.g., and the like. In one embodiment, these promoters are altered at their downstream intron containing end to include one or more nuclease cleavage sites. In one embodiment, the DNA containing the nuclease cleavage site(s) is foreign to the promoter DNA.

In one embodiment, a promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to the cell, tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter, as well as the promoters listed below. Such promoters and/or enhancers can be used for expression of any gene of interest, e.g., therapeutic proteins). For example, the vector may comprise a promoter that is operably linked to the nucleic acid sequence encoding a therapeutic protein. In one embodiment, the promoter operably linked to the therapeutic protein coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. In one embodiment, the promoter may also be a promoter from a human gene such as human ubiquitin C (hUbC), human actin, human myosin, human hemoglobin, human muscle creatine, or human metallothionein. The promoter may also be a tissue specific promoter, such as a liver specific promoter, such as human alpha 1-antitypsin (HAAT), natural or synthetic. In one embodiment, delivery to the liver can be achieved using endogenous ApoE specific targeting of the composition comprising a ceDNA vector to hepatocytes via the low density lipoprotein (LDL) receptor present on the surface of the hepatocyte.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e.g., enhancers.

Non-limiting examples of suitable promoters for use in accordance with the present invention include the CAG promoter of, for example, the HAAT promoter, the human EF1-α promoter or a fragment of the EF1αα promoter and the rat EF1-α promoter.

### Polyadenylation Sequences

A sequence encoding a polyadenylation sequence can be included in the ceDNA vector to stabilize the mRNA expressed from the ceDNA vector, and to aid in nuclear export and translation. In one embodiment, the ceDNA vector does not include a polyadenylation sequence. In other embodiments, the vector includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between.

In one embodiment, the ceDNA can be obtained from a vector polynucleotide that encodes a heterologous nucleic acid operatively positioned between two different inverted terminal repeat sequences (ITRs) (e.g. AAV ITRs), wherein at least one of the ITRs comprises a terminal resolution site and a replicative protein binding site (RPS), e.g. a Rep binding site (e.g. wt AAV ITR), and one of the ITRs comprises a deletion, insertion, and/or substitution with respect to the other ITR, e.g., functional ITR.

In one embodiment, the host cells do not express viral capsid proteins and the polynucleotide vector template is devoid of any viral capsid coding sequences. In one embodiment, the polynucleotide vector template is devoid of AAV capsid genes but also of capsid genes of other viruses). In one embodiment, the nucleic acid molecule is also devoid of AAV Rep protein coding sequences. Accordingly, in some embodiments, the nucleic acid molecule of the invention is devoid of both functional AAV cap and AAV rep genes.

In one embodiment, the ceDNA vector does not have a modified ITRs.

In one embodiment, the ceDNA vector comprises a regulatory switch as disclosed herein (or in PCT application No. PCT/US 18/49996, filed Sep. 7, 2018).

### V. Production of a ceDNA Vector

Methods for the production of a ceDNA vector as described herein comprising an asymmetrical ITR pair or symmetrical ITR pair as defined herein is described in section IV of PCT/US 18/49996 filed Sep. 7, 2018, which is incorporated herein in its entirety by reference. As described herein, the ceDNA vector can be obtained, for example, by the process comprising the steps of: a) incubating a population of host cells (e.g. insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell.

However, no viral particles (e.g. AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In one embodiment, the invention provides for use of host cell lines that have stably integrated the DNA vector polynucleotide expression template (ceDNA template) into their own genome in production of the non-viral DNA vector, e.g. as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

In one embodiment, the host cells used to make the ceDNA vectors described herein are insect cells, and baculovirus is used to deliver both the polynucleotide that encodes Rep protein and the non-viral DNA vector polynucleotide expression construct template for ceDNA. In some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce ceDNA vectors but before a majority of cells start to die because of the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. In one embodiment, ceDNA vectors are purified as DNA molecules. In one embodiment, the ceDNA vectors are purified as exosomes or microparticles. The presence of the ceDNA vector can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA.

ceDNA Plasmid

A ceDNA-plasmid is a plasmid used for later production of a ceDNA vector. In one embodiment, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes. In one embodiment, a ceDNA vector is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs are have the same modifications (i.e., they are inverse complement or symmetric relative to each other).

In one embodiment, the ceDNA-plasmid system is devoid of viral capsid protein coding sequences (i.e. it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation. In one embodiment, a ceDNA-plasmid of the present disclosure can be generated using natural nucleotide sequences of the genomes of any AAV serotypes well known in the art. In one embodiment, the ceDNA-plasmid backbone is derived from the AAV1, AAV2, AAV3, AAV4, AAV5, AAV 5, AAV7, AAV8, AAV9, AAV 10, AAV 11, AAV 12, AAVrh8, AAVrhlO, AAV-DJ, and AAV-DJ8 genome, e.g., NCBI: NC 002077; NC 001401; NC001729; NC001829; NC006152; NC 006260; NC 006261; Kotin and Smith, The Springer Index of Viruses, available at the URL maintained by Springer. In one embodiment, the ceDNA-plasmid backbone is derived from the AAV2 genome. In one embodiment, the ceDNA-plasmid backbone is a synthetic backbone genetically engineered to include at its 5′ and 3′ ITRs derived from one of these AAV genomes.

In one embodiment, a ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

In one embodiment, an Exemplary ceDNA (e.g., rAAVO) is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

Exemplary Method of Making the ceDNA Vectors from ceDNA Plasmids

In one embodiment, methods for making capsid-less ceDNA vectors are also provided herein, notably a method with a sufficiently high yield to provide sufficient vector for in vivo experiments.

In one embodiment, a method for the production of a ceDNA vector comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of ceDNA vector can be in the form of a ceDNA plasmid, or Bacmid or Baculovirus generated with the ceDNA plasmid as described below. The nucleic acid construct can be introduced into a host cell by transfection, viral transduction, stable integration, or other methods known in the art.

### Cell Lines

In one embodiment, host cell lines used in the production of a ceDNA vector can include insect cell lines derived from Spodoptera frugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, He La, HepG2, Hep1A, 911, CHO, COS, MeWo, NIH3T3, A549, HT1 180, monocytes, and mature and immature dendritic cells. Host cell lines can be transfected for stable expression of the ceDNA-plasmid for high yield ceDNA vector production.

In one embodiment, ceDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA—plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

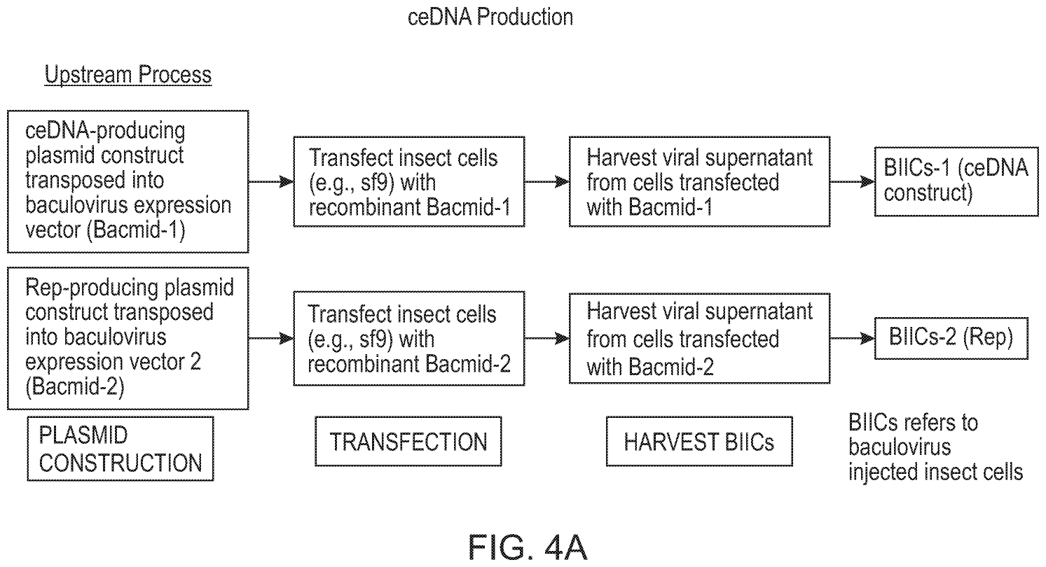

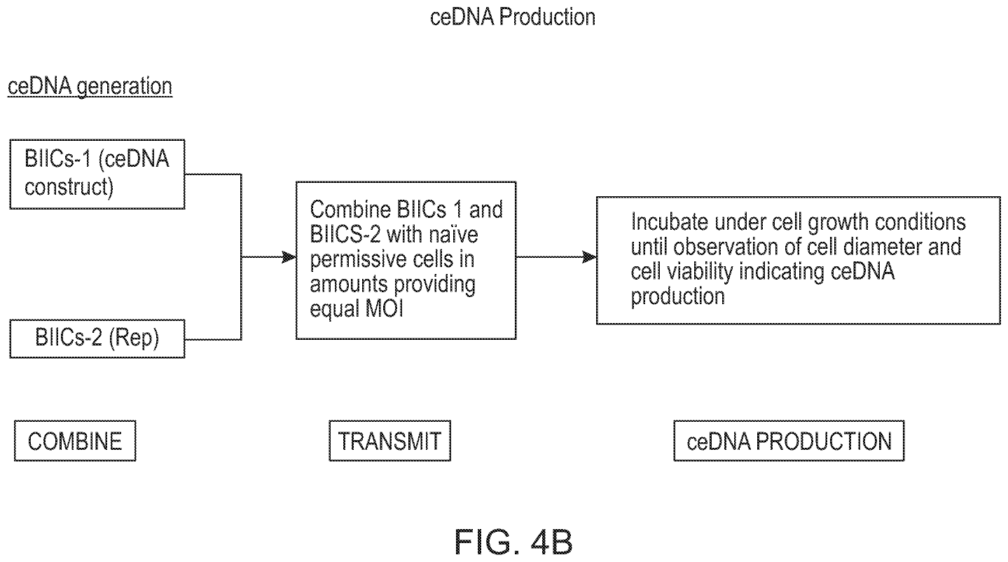

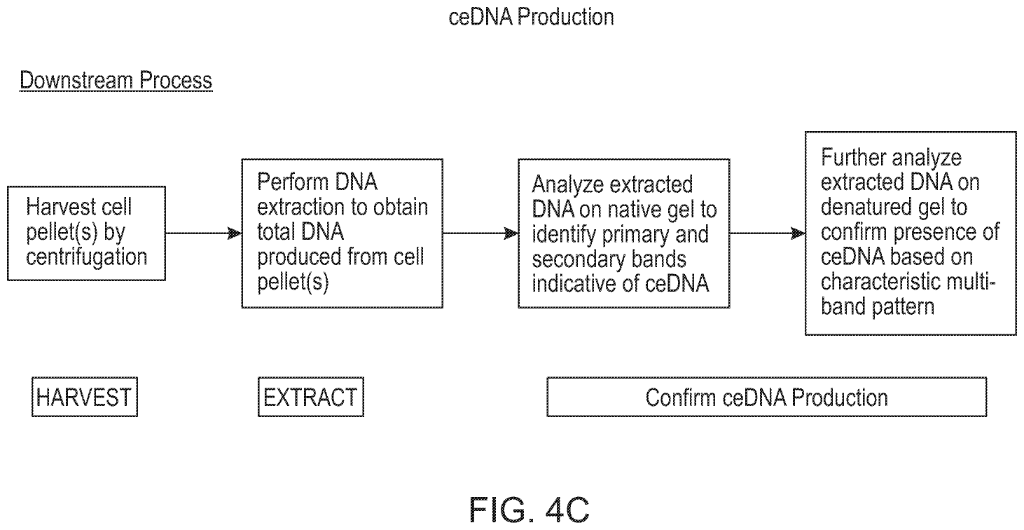

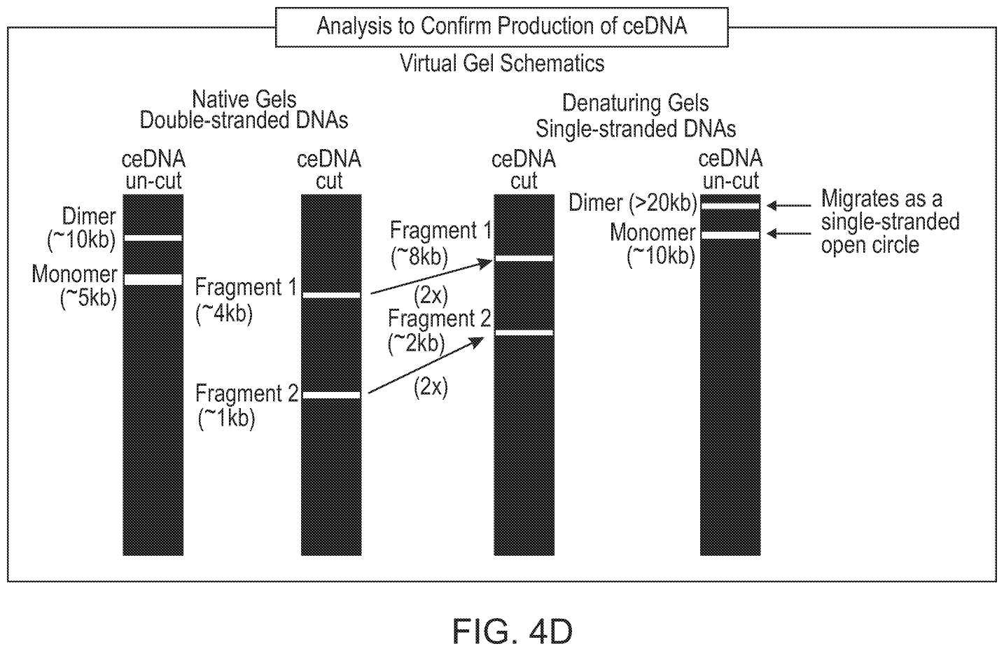

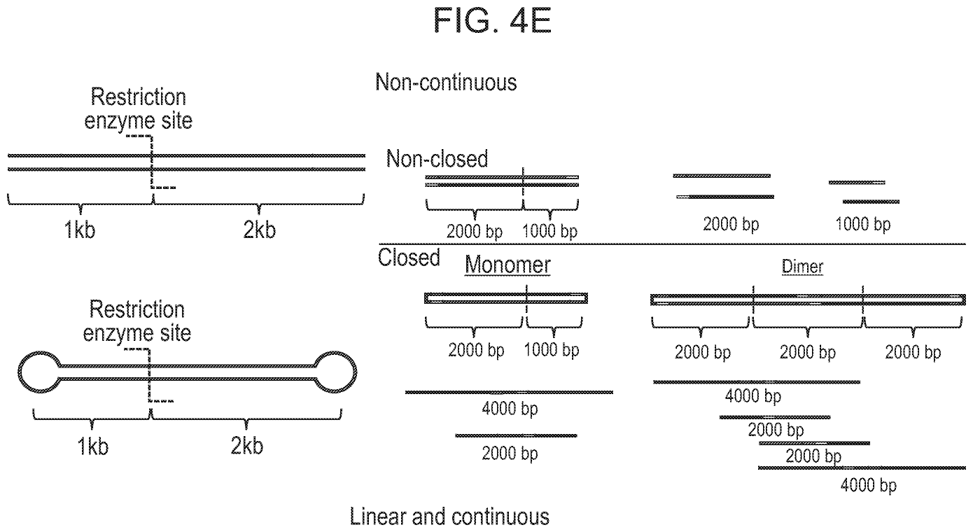

Isolating and Purifying ceDNA vectors Examples of the process for obtaining and isolating ceDNA vectors (e.g. for gene editing) are described in FIGS. 4A-4E of International Application No. PCT/US2018/064242, filed Dec. 6, 2018, the contents of which is incorporated by reference in its entirety herein. In one embodiment, ceDNA-vectors can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids shown in FIG. 6A (useful for Rep BIICs production), FIG. 6B (plasmid used to obtain a ceDNA vector) of International Application No. PCT/US2018/064242.

In one embodiment, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA-vector, which is an exemplary ceDNA vector, are described herein. Expression constructs used for generating a ceDNA vectors of the present invention can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors can be generated from the cells stably transfected with a construct comprising a sequence encoding the AAV Rep protein (Rep78/52) delivered in Rep-plasmids, Rep-bacmids, or Rep-baculovirus. CeDNA-Baculovirus can be transiently transfected to the cells, be replicated by Rep protein and produce ceDNA vectors.

The bacmid (e.g., ceDNA-bacmid) can be transfected into a permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one nonlimiting example, the process can be performed by loading the supernatant on an ion exchange column (e.g., SARTOBIND Q®) which retains nucleic acids, and then eluting (e.g. with a 1.2 M NaCl solution) and performing a further chromatographic purification on a gel filtration column (e.g., 6 fast flow GE). The capsid-free AAV vector is then recovered by, e.g., precipitation.

In one embodiment, ceDNA vectors can also be purified in the form of exosomes, or microparticles. It is known in the art that many cell types release not only soluble proteins, but also complex protein/nucleic acid cargoes via membrane microvesicle shedding (Cocucci et al, 2009; EP 10306226.1). Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid. In one embodiment, microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226), incorporated by reference in its entirety herein.

Another aspect of the invention relates to methods of purifying ceDNA vectors from host cell lines that have stably integrated a ceDNA construct into their own genome. In one embodiment, ceDNA vectors are purified as DNA molecules. In another embodiment, the ceDNA vectors are purified as exosomes or microparticles.

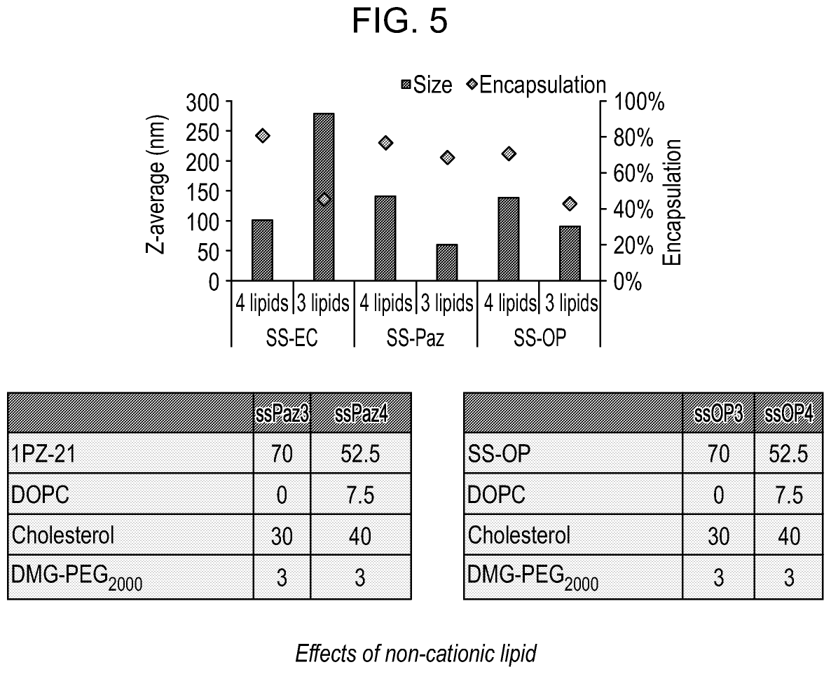

FIG. 5 of PCT/US 18/49996 shows a gel confirming the production of ceDNA from multiple ceDNA-plasmid constructs using the method described in the Examples.

### VI. Preparation of Lipid Particles

Lipid particles (e.g., lipid nanoparticles) can form spontaneously upon mixing of ceDNA and the lipid(s). Depending on the desired particle size distribution, the resultant nanoparticle mixture can be extruded through a membrane (e.g., 100 nrn cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. In one embodiment, the lipid nanoparticles are formed as described in Example 6 herein.

Generally, lipid particles (e.g., lipid nanoparticles) can be formed by any method known in the art. For example, the lipid particles (e.g., lipid nanoparticles) can be prepared by the methods described, for example, in US2013/0037977, US2010/0015218, US2013/0156845, US2013/0164400, US2012/0225129, and US2010/0130588, content of each of which is incorporated herein by reference in its entirety. In some embodiments, lipid particles (e.g., lipid nanoparticles) can be prepared using a continuous mixing method, a direct dilution process, or an in-line dilution process. The processes and apparatuses for apparatuses for preparing lipid nanoparticles using direct dilution and in-line dilution processes are described in US2007/0042031, the content of which is incorporated herein by reference in its entirety. The processes and apparatuses for preparing lipid nanoparticles using step-wise dilution processes are described in US2004/0142025, the content of which is incorporated herein by reference in its entirety.

In one embodiment, the lipid particles (e.g., lipid nanoparticles) can be prepared by an impinging jet process. Generally, the particles are formed by mixing lipids dissolved in alcohol (e.g., ethanol) with ceDNA dissolved in a buffer, e.g., a citrate buffer, a sodium acetate buffer, a sodium acetate and magnesium chloride buffer, a malic acid buffer, a malic acid and sodium chloride buffer, or a sodium citrate and sodium chloride buffer. The mixing ratio of lipids to ceDNA can be about 45-55% lipid and about 65-45% ceDNA.

The lipid solution can contain a cationic lipid (e.g. an ionizable cationic lipid), a non-cationic lipid (e.g., a phospholipid, such as DSPC, DOPE, and DOPC), PEG or PEG conjugated molecule (e.g., PEG-lipid), and a sterol (e.g., cholesterol) at a total lipid concentration of 5-30 mg/mL, more likely 5-15 mg/mL, most likely 9-12 mg/mL in an alcohol, e.g., in ethanol. In the lipid solution, mol ratio of the lipids can range from about 25-98% for the cationic lipid, preferably about 35-65%; about 0-15% for the non-ionic lipid, preferably about 0-12%; about 0-15% for the PEG or PEG conjugated lipid molecule, preferably about 1-6%; and about 0-75% for the sterol, preferably about 30-50%.

The ceDNA solution can comprise the ceDNA at a concentration range from 0.3 to 1.0 mg/mL, preferably 0.3-0.9 mg/mL in buffered solution, with pH in the range of 3.5-5.

For forming the LNPs, in one exemplary but nonlimiting embodiment, the two liquids are heated to a temperature in the range of about 15-40° C., preferably about 30-40° C., and then mixed, for example, in an impinging jet mixer, instantly forming the LNP. The mixing flow rate can range from 10-600 mL/min. The tube ID can have a range from 0.25 to 1.0 mm and a total flow rate from 10-600 mL/min. The combination of flow rate and tubing ID can have the effect of controlling the particle size of the LNPs between 30 and 200 nm. The solution can then be mixed with a buffered solution at a higher pH with a mixing ratio in the range of 1:1 to 1:3 vol:vol, preferably about 1:2 vol:vol. If needed this buffered solution can be at a temperature in the range of 15-40° C. or 30-40° C. The mixed LNPs can then undergo an anion exchange filtration step. Prior to the anion exchange, the mixed LNPs can be incubated for a period of time, for example 30 mins to 2 hours. The temperature during incubating can be in the range of 15-40° C. or 30-40° C. After incubating the solution is filtered through a filter, such as a 0.8 μm filter, containing an anion exchange separation step. This process can use tubing IDs ranging from 1 mm ID to 5 mm ID and a flow rate from 10 to 2000 mL/min.

After formation, the LNPs can be concentrated and diafiltered via an ultrafiltration process where the alcohol is removed and the buffer is exchanged for the final buffer solution, for example, phosphate buffered saline (PBS) at about pH 7, e.g., about pH 6.9, about pH 7.0, about pH 7.1, about pH 7.2, about pH 7.3, or about pH 7.4.

The ultrafiltration process can use a tangential flow filtration format (TFF) using a membrane nominal molecular weight cutoff range from 30-500 kD. The membrane format is hollow fiber or flat sheet cassette. The TFF processes with the proper molecular weight cutoff can retain the LNP in the retentate and the filtrate or permeate contains the alcohol; citrate buffer and final buffer wastes. The TFF process is a multiple step process with an initial concentration to a ceDNA concentration of 1-3 mg/mL. Following concentration, the LNPs solution is diafiltered against the final buffer for 10-20 volumes to remove the alcohol and perform buffer exchange. The material can then be concentrated an additional 1-3-fold. The concentrated LNP solution can be sterile filtered.

### VII. Pharmaceutical Compositions and Formulations

Also provided herein is a pharmaceutical composition comprising the ceDNA lipid particle and a pharmaceutically acceptable carrier or excipient.

In one embodiment, the ceDNA lipid particles (e.g., lipid nanoparticles) are provided with full encapsulation, partial encapsulation of the therapeutic nucleic acid. In one embodiment, the nucleic acid therapeutics is fully encapsulated in the lipid particles (e.g., lipid nanoparticles) to form a nucleic acid containing lipid particle. In one embodiment, the nucleic acid may be encapsulated within the lipid portion of the particle, thereby protecting it from enzymatic degradation.

In one embodiment, the lipid particle has a mean diameter from about 20 nm to about 100 nm, 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm to ensure effective delivery. Nucleic acid containing lipid particles (e.g., lipid nanoparticles) and their method of preparation are disclosed in, e.g., PCT/US18/50042, U.S. Patent Publication Nos. 20040142025 and 20070042031, the disclosures of which are herein incorporated by reference in their entirety for all purposes. In one embodiment, lipid particle (e.g., lipid nanoparticle) size can be determined by quasi-elastic light scattering using, for example, a Malvern Zetasizer Nano ZS (Malvern, UK) system.

Generally, the lipid particles (e.g., lipid nanoparticles) of the invention have a mean diameter selected to provide an intended therapeutic effect.

Depending on the intended use of the lipid particles (e.g., lipid nanoparticles), the proportions of the components can be varied and the delivery efficiency of a particular formulation can be measured using, for example, an endosomal release parameter (ERP) assay.

In one embodiment, the lipid particles (e.g., lipid nanoparticles) may be conjugated with other moieties to prevent aggregation. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

In one embodiment, the ceDNA can be complexed with the lipid portion of the particle or encapsulated in the lipid position of the lipid particle (e.g., lipid nanoparticle). In one embodiment, the ceDNA can be fully encapsulated in the lipid position of the lipid particle (e.g., lipid nanoparticle), thereby protecting it from degradation by a nuclease, e.g., in an aqueous solution. In one embodiment, the ceDNA in the lipid particle (e.g., lipid nanoparticle) is not substantially degraded after exposure of the lipid particle (e.g., lipid nanoparticle) to a nuclease at 37° C. for at least about 20, 30, 45, or 60 minutes. In some embodiments, the ceDNA in the lipid particle (e.g., lipid nanoparticle) is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In one embodiment, the lipid particles (e.g., lipid nanoparticles) are substantially non-toxic to a subject, e.g., to a mammal such as a human.

In one embodiment, a pharmaceutical composition comprising a therapeutic nucleic acid of the present disclosure may be formulated in lipid particles (e.g., lipid nanoparticles). In some embodiments, the lipid particle comprising a therapeutic nucleic acid can be formed from a cationic lipid. In some other embodiments, the lipid particle comprising a therapeutic nucleic acid can be formed from non-cationic lipid. In a preferred embodiment, the lipid particle of the invention is a nucleic acid containing lipid particle, which is formed from a cationic lipid comprising a therapeutic nucleic acid selected from the group consisting of mRNA, antisense RNA and oligonucleotide, ribozymes, aptamer, interfering RNAs (RNAi), Dicer-substrate dsRNA, small hairpin RNA (shRNA), asymmetrical interfering RNA (aiRNA), microRNA (miRNA), minicircle DNA, minigene, viral DNA (e.g., Lentiviral or AAV genome) or non-viral synthetic DNA vectors, closed-ended linear duplex DNA (ceDNA/CELiD), plasmids, bacmids, Doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

In another preferred embodiment, the lipid particle of the invention is a nucleic acid containing lipid particle, which is formed from a non-cationic lipid, and optionally a conjugated lipid that prevents aggregation of the particle.

In one embodiment, the lipid particle formulation is an aqueous solution. In one embodiment, the lipid particle (e.g., lipid nanoparticle) formulation is a lyophilized powder.

According to some aspects, the disclosure provides for a lipid particle formulation further comprising one or more pharmaceutical excipients. In one embodiment, the lipid particle (e.g., lipid nanoparticle) formulation further comprises sucrose, tris, trehalose and/or glycine.

In one embodiment, the lipid particles (e.g., lipid nanoparticles) disclosed herein can be incorporated into pharmaceutical compositions suitable for administration to a subject for in vivo delivery to cells, tissues, or organs of the subject. Typically, the pharmaceutical composition comprises the ceDNA lipid particles (e.g., lipid nanoparticles) disclosed herein and a pharmaceutically acceptable carrier. In one embodiment, the ceDNA lipid particles (e.g., lipid nanoparticles) of the disclosure can be incorporated into a pharmaceutical composition suitable for a desired route of therapeutic administration (e.g., parenteral administration). Passive tissue transduction via high pressure intravenous or intraarterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable for high ceDNA vector concentration. Sterile injectable solutions can be prepared by incorporating the ceDNA vector compound in the required amount in an appropriate buffer with one or a combination of ingredients enumerated above, as required, followed by filtered sterilization.

A lipid particle as disclosed herein can be incorporated into a pharmaceutical composition suitable for topical, systemic, intra-amniotic, intrathecal, intracranial, intraarterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

Pharmaceutically active compositions comprising ceDNA lipid particles (e.g., lipid nanoparticles) can be formulated to deliver a transgene in the nucleic acid to the cells of a recipient, resulting in the therapeutic expression of the transgene therein. The composition can also include a pharmaceutically acceptable carrier.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

In one embodiment, lipid particles (e.g., lipid nanoparticles) are solid core particles that possess at least one lipid bilayer. In one embodiment, the lipid particles (e.g., lipid nanoparticles) have a non-bilayer structure, i.e., a non-lamellar (i.e., non-bilayer) morphology. Without limitations, the non-bilayer morphology can include, for example, three dimensional tubes, rods, cubic symmetries, etc. The non-lamellar morphology (i.e., non-bilayer structure) of the lipid particles (e.g. lipid nanoparticles) can be determined using analytical techniques known to and used by those of skill in the art. Such techniques include, but are not limited to, Cryo-Transmission Electron Microscopy (“Cryo-TEM”), Differential Scanning calorimetry (“DSC”), X-Ray Diffraction, and the like. For example, the morphology of the lipid particles (lamellar vs. non-lamellar) can readily be assessed and characterized using, e.g., Cryo-TEM analysis as described in US2010/0130588, the content of which is incorporated herein by reference in its entirety.

In one embodiment, the lipid particles (e.g., lipid nanoparticles) having a non-lamellar morphology are electron dense.

In one embodiment, the disclosure provides for a lipid particle (e.g., lipid nanoparticle) that is either unilamellar or multilamellar in structure. In some aspects, the disclosure provides for a lipid particle (e.g., lipid nanoparticle) formulation that comprises multi-vesicular particles and/or foam-based particles. By controlling the composition and concentration of the lipid components, one can control the rate at which the lipid conjugate exchanges out of the lipid particle and, in turn, the rate at which the lipid particle (e.g., lipid nanoparticle) becomes fusogenic. In addition, other variables including, for example, pH, temperature, or ionic strength, can be used to vary and/or control the rate at which the lipid particle (e.g., lipid nanoparticle) becomes fusogenic. Other methods which can be used to control the rate at which the lipid particle (e.g., lipid nanoparticle) becomes fusogenic will be apparent to those of ordinary skill in the art based on this disclosure. It will also be apparent that by controlling the composition and concentration of the lipid conjugate, one can control the lipid particle size.

In one embodiment, the pKa of formulated cationic lipids can be correlated with the effectiveness of the LNPs for delivery of nucleic acids (see Jayaraman et al., Angewandte Chemie, International Edition (2012), 51(34), 8529-8533; Semple et al., Nature Biotechnology 28, 172-176 (2010), both of which are incorporated by reference in their entireties). In one embodiment, the preferred range of pKa is ˜5 to ˜7. In one embodiment, the pKa of the cationic lipid can be determined in lipid particles (e.g., lipid nanoparticles) using an assay based on fluorescence of 2-(p-toluidino)-6-napthalene sulfonic acid (TNS).

In one embodiment, encapsulation of ceDNA in lipid particles (e.g. lipid nanoparticles) can be determined by performing a membrane-impermeable fluorescent dye exclusion assay, which uses a dye that has enhanced fluorescence when associated with nucleic acid, for example, an Oligreen® assay or PicoGreen® assay. Generally, encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated ceDNA, allowing it to interact with the membrane-impermeable dye. Encapsulation of ceDNA can be calculated as E=(Io−I)/Io, where I and Io refers to the fluorescence intensities before and after the addition of detergent.

### Unit Dosage

In one embodiment, the pharmaceutical compositions can be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### VIII. Methods of Treatment

The ceDNA vectors (e.g., ceDNA vector lipid particles as described herein) and compositions described herein can be used to introduce a nucleic acid sequence (e.g., a therapeutic nucleic acid sequence) in a host cell. In one embodiment, introduction of a nucleic acid sequence in a host cell using the ceDNA vectors (e.g., ceDNA vector lipid particles as described herein) can be monitored with appropriate biomarkers from treated patients to assess gene expression.

The compositions and vectors provided herein can be used to deliver a transgene (a nucleic acid sequence) for various purposes. In one embodiment, the ceDNA vectors (e.g., ceDNA vector lipid particles as described herein) can be used in a variety of ways, including, for example, ex situ, in vitro and in vivo applications, methodologies, diagnostic procedures, and/or gene therapy regimens.

Provided herein are methods of treating a disease or disorder in a subject comprising introducing into a target cell in need thereof (for example, a muscle cell or tissue, or other affected cell type) of the subject a therapeutically effective amount of a ceDNA vector (e.g., ceDNA vector lipid particles as described herein), optionally with a pharmaceutically acceptable carrier. While the ceDNA vector (e.g., ceDNA vector lipid particles as described herein) can be introduced in the presence of a carrier, such a carrier is not required. The ceDNA vector (e.g., ceDNA vector lipid particles as described herein) implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector (e.g., ceDNA vector lipid particles as described herein) can be administered via any suitable route as described herein and known in the art. In one embodiment, the target cells are in a human subject.

Provided herein are methods for providing a subject in need thereof with a diagnostically- or therapeutically-effective amount of a ceDNA vector (e.g., ceDNA vector lipid particles as described herein), the method comprising providing to a cell, tissue or organ of a subject in need thereof, an amount of the ceDNA vector (e.g., ceDNA vector lipid particles as described herein); and for a time effective to enable expression of the transgene from the ceDNA vector thereby providing the subject with a diagnostically- or a therapeutically-effective amount of the protein, peptide, nucleic acid expressed by the ceDNA vector (e.g., ceDNA vector lipid particles as described herein). In one embodiment, the subject is human.

Provided herein are methods for diagnosing, preventing, treating, or ameliorating at least one or more symptoms of a disease, a disorder, a dysfunction, an injury, an abnormal condition, or trauma in a subject. Generally, the method includes at least the step of administering to a subject in need thereof one or more ceDNA vectors (e.g., ceDNA vector lipid particles as described herein), in an amount and for a time sufficient to diagnose, prevent, treat or ameliorate the one or more symptoms of the disease, disorder, dysfunction, injury, abnormal condition, or trauma in the subject. In one embodiment, the subject is human.

Provided herein are methods comprising using of the ceDNA vector as a tool for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, ceDNA vectors (e.g., ceDNA vector lipid particles as described herein) can be used to deliver transgenes to bring a normal gene into affected tissues for replacement therapy, as well, in some embodiments, to create animal models for the disease using antisense mutations. For unbalanced disease states, ceDNA vectors (e.g., ceDNA vector lipid particles as described herein) can be used to create a disease state in a model system, which could then be used in efforts to counteract the disease state. Thus, the ceDNA vectors (e.g., ceDNA vector lipid particles as described herein) and methods disclosed herein permit the treatment of genetic diseases. As used herein, a disease state is treated by partially or wholly remedying the deficiency or imbalance that causes the disease or makes it more severe.

In general, the ceDNA vector (e.g., ceDNA vector lipid particles as described herein) can be used to deliver any transgene in accordance with the description above to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, the ceDNA vectors as disclosed herein can be advantageously used in the treatment of individuals with metabolic disorders (e.g., ornithine transcarbamylase deficiency).

In one embodiment, the ceDNA vector described herein can be used to treat, ameliorate, and/or prevent a disease or disorder caused by mutation in a gene or gene product. Exemplary diseases or disorders that can be treated with ceDNA vectors (e.g., ceDNA vector lipid particles (e.g., lipid nanoparticles) as described herein)s include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia (A and B), thalassemia, and anemia); cancers and tumors, and genetic diseases or disorders (e.g., cystic fibrosis).

In one embodiment, ceDNA vectors (e.g., a ceDNA vector lipids particle as described herein) may be employed to deliver a heterologous nucleotide sequence in situations in which it is desirable to regulate the level of transgene expression (e.g., transgenes encoding hormones or growth factors, as described herein).

In one embodiment, the ceDNA vectors (e.g., a ceDNA vector lipid particles as described herein) can be used to correct an abnormal level and/or function of a gene product (e.g., an absence of, or a defect in, a protein) that results in the disease or disorder. The ceDNA vectors (e.g., ceDNA vector lipid particles as described herein) can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MFD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In one embodiment, the ceDNA vectors (e.g., ceDNA vector lipid particles as described herein) can be used to provide an RNA-based therapeutic to a cell in vitro or in vivo. Examples of RNA-based therapeutics include, but are not limited to, mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). For example, in one embodiment, the ceDNA vectors (e.g., ceDNA vector lipid particles as described herein) can be used to provide an antisense nucleic acid to a cell in vitro or in vivo. For example, where the transgene is a RNAi molecule, expression of the antisense nucleic acid or RNAi in the target cell diminishes expression of a particular protein by the cell. Accordingly, transgenes which are RNAi molecules or antisense nucleic acids may be administered to decrease expression of a particular protein in a subject in need thereof. Antisense nucleic acids may also be administered to cells in vitro to regulate cell physiology, e.g., to optimize cell or tissue culture systems.

In one embodiment, the ceDNA vectors (e.g., ceDNA vector lipid particles as described herein) can be used to provide a DNA-based therapeutic to a cell in vitro or in vivo. Examples of DNA-based therapeutics include, but are not limited to, minicircle DNA, minigene, viral DNA (e.g., Lentiviral or AAV genome) or non-viral synthetic DNA vectors, closed-ended linear duplex DNA (ceDNA/CELiD), plasmids, bacmids, Doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”). For example, in one embodiment, the ceDNA vectors (e.g., ceDNA vector lipid particles as described herein) can be used to provide minicircle to a cell in vitro or in vivo. For example, where the transgene is a minicircle DNA, expression of the minicircle DNA in the target cell diminishes expression of a particular protein by the cell. Accordingly, transgenes which are minicircle DNAs may be administered to decrease expression of a particular protein in a subject in need thereof. Minicircle DNAs may also be administered to cells in vitro to regulate cell physiology, e.g., to optimize cell or tissue culture systems.

In one embodiment, exemplary transgenes encoded by the ceDNA vector include, but are not limited to: X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, b-interferon, interferon-g, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-a and -b, and the like), receptors (e.g., tumor necrosis factor receptor). In some exemplary embodiments, the transgene encodes a monoclonal antibody specific for one or more desired targets. In some exemplary embodiments, more than one transgene is encoded by the ceDNA vector. In some exemplary embodiments, the transgene encodes a fusion protein comprising two different polypeptides of interest. In some embodiments, the transgene encodes an antibody, including a full-length antibody or antibody fragment, as defined herein. In some embodiments, the antibody is an antigen-binding domain or an immunoglobulin variable domain sequence, as that is defined herein. Other illustrative transgene sequences encode suicide gene products (thymidine kinase, cytosine deaminase, diphtheria toxin, cytochrome P450, deoxycytidine kinase, and tumor necrosis factor), proteins conferring resistance to a drug used in cancer therapy, and tumor suppressor gene products.

### Administration

In one embodiment, a ceDNA vector (e.g., a ceDNA vector lipid particle as described herein) can be administered to an organism for transduction of cells in vivo. In one embodiment, ceDNA vectors (e.g., ceDNA vector lipid particles as described herein) can be administered to an organism for transduction of cells ex vivo.

Generally, administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Exemplary modes of administration of the ceDNA vectors (e.g., ceDNA vector lipid particles as described herein) includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular (including administration to skeletal, diaphragm and/or cardiac muscle), intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the ceDNA vector (e.g., a ceDNA vector lipid particle as described herein) can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. In one embodiment, administration of the ceDNA vectors (e.g., ceDNA vector lipid particles as described herein) can also be to a tumor (e.g., in or near a tumor or a lymph node). The most suitable route in any given case will depend on the nature and severity of the condition being treated, ameliorated, and/or prevented and on the nature of the particular ceDNA vectors (e.g., ceDNA vector lipid particles as described herein) that is being used. Additionally, ceDNA permits one to administer more than one transgene in a single vector, or multiple ceDNA vectors (e.g. a ceDNA cocktail).

In one embodiment, administration of the ceDNA vectors (e.g., ceDNA vector lipid particles as described herein) to skeletal muscle includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The ceDNA vectors (e.g., ceDNA vector lipid particles as described herein) can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the ceDNA vector (e.g., a ceDNA vector lipid particle as described herein) is administered to a limb (arm and/or leg) of a subject (e.g., a subject with muscular dystrophy such as DMD) by limb perfusion, optionally isolated limb perfusion (e.g., by intravenous or intra-articular administration. In one embodiment, the ceDNA vector (e.g., a ceDNA vector lipid particle as described herein) can be administered without employing “hydrodynamic” techniques.

Administration of the ceDNA vectors (e.g., a ceDNA vector lipid particles as described herein) to cardiac muscle includes administration to the left atrium, right atrium, left ventricle, right ventricle and/or septum. The ceDNA vectors (e.g., ceDNA vector lipid particles as described herein) can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In one embodiment, ceDNA vectors (e.g., ceDNA vector lipid particles as described herein) are administered to skeletal muscle, diaphragm muscle and/or cardiac muscle (e.g., to treat, ameliorate, and/or prevent muscular dystrophy or heart disease (e.g., PAD or congestive heart failure).

ceDNA vectors (e.g., ceDNA vector lipid particles as described herein) can be administered to the CNS (e.g., to the brain or to the eye). The ceDNA vectors (e.g., ceDNA vector lipid particles as described herein) may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vectors (e.g., ceDNA vector lipid particles (e.g., lipid nanoparticles) as described herein) may also be administered to different regions of the eye such as the retina, cornea and/or optic nerve. The ceDNA vectors (e.g., ceDNA vector lipid particles (e.g., lipid nanoparticles) as described herein) may be delivered into the cerebrospinal fluid (e.g., by lumbar puncture). The ceDNA vectors (e.g., ceDNA vector lipid particles (e.g., lipid nanoparticles) as described herein) may further be administered intravascularly to the CNS in situations in which the blood-brain barrier has been perturbed (e.g., brain tumor or cerebral infarct).

In one embodiment, the ceDNA vectors (e.g., ceDNA vector lipid particles as described herein) can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and pen-ocular (e.g., sub-Tenon's region) delivery as well as intramuscular delivery with retrograde delivery to motor neurons.

According to some embodiment, the ceDNA vectors (e.g., ceDNA vector lipid particles as described herein) is administered in a liquid formulation by direct injection (e.g., stereotactic injection) to the desired region or compartment in the CNS. According to other embodiments, the ceDNA vectors (e.g., ceDNA vector lipid particles as described herein) can be provided by topical application to the desired region or by intra-nasal administration of an aerosol formulation. Administration to the eye may be by topical application of liquid droplets. As a further alternative, the ceDNA vector can be administered as a solid, slow-release formulation (see, e.g., U.S. Pat. No. 7,201,898, incorporated by reference in its entirety herein). In one embodiment, the ceDNA vectors (e.g., ceDNA vector lipid particles as described herein) can used for retrograde transport to treat, ameliorate, and/or prevent diseases and disorders involving motor neurons (e.g., amyotrophic lateral sclerosis (ALS); spinal muscular atrophy (SMA), etc.). For example, the ceDNA vectors (e.g., ceDNA vector lipid particles as described herein) can be delivered to muscle tissue from which it can migrate into neurons.

In one embodiment, repeat administrations of the therapeutic product can be made until the appropriate level of expression has been achieved. Thus, in one embodiment, a therapeutic nucleic acid can be administered and re-dosed multiple times. For example, the therapeutic nucleic acid can be administered on day 0. Following the initial treatment at day 0, a second dosing (re-dose) can be performed in about 1 week, about 2 weeks, about 3 weeks, about 4 weeks, about 5 weeks, about 6 weeks, about 7 weeks, about 8 weeks, or about 3 months, about 4 months, about 5 months, about 6 months, about 7 months, about 8 months, about 9 months, about 10 months, about 11 months, or about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years or about 50 years after the initial treatment with the therapeutic nucleic acid.

In one embodiment, one or more additional compounds can also be included. Those compounds can be administered separately or the additional compounds can be included in the lipid particles (e.g., lipid nanoparticles) of the invention. In other words, the lipid particles (e.g., lipid nanoparticles) can contain other compounds in addition to the ceDNA or at least a second ceDNA, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In one embodiment, the one or more additional compound can be a therapeutic agent. The therapeutic agent can be selected from any class suitable for the therapeutic objective. Accordingly, the therapeutic agent can be selected from any class suitable for the therapeutic objective. The therapeutic agent can be selected according to the treatment objective and biological action desired. For example, In one embodiment, if the ceDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate).

In one embodiment, if the LNP containing the ceDNA is useful for treating an infection, the additional compound can be an antimicrobial agent (e.g., an antibiotic or antiviral compound). In one embodiment, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In one embodiment, different cocktails of different lipid particles containing different compounds, such as a ceDNA encoding a different protein or a different compound, such as a therapeutic may be used in the compositions and methods of the invention. In one embodiment, the additional compound is an immune modulating agent. For example, the additional compound is an immunosuppressant. In some embodiments, the additional compound is immunostimulatory.

### EXAMPLES

The following examples are provided by way of illustration not limitation. It will be appreciated by one of ordinary skill in the art that ceDNA vectors can be constructed from any of the wild-type or modified ITRs described herein, and that the following exemplary methods can be used to construct and assess the activity of such ceDNA vectors. While the methods are exemplified with certain ceDNA vectors, they are applicable to any ceDNA vector in keeping with the description.

### Example 1: Constructing ceDNA Vectors Using an Insect Cell-Based Method

Production of the ceDNA vectors using a polynucleotide construct template is described in Example 1 of PCT/US18/49996, which is incorporated herein in its entirety by reference. For example, a polynucleotide construct template used for generating the ceDNA vectors of the present invention can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g., ceDNA-plasmid, ceDNA-bacmid, ceDNA-baculovirus genome etc.) via Rep proteins, and second, Rep mediated replication of the excised ceDNA vector.

An exemplary method to produce ceDNA vectors is from a ceDNA-plasmid as described herein. Referring to FIGS. 1A and 1B, the polynucleotide construct template of each of the ceDNA-plasmids includes both a left modified ITR and a right modified ITR with the following between the ITR sequences: (i) an enhancer/promoter; (ii) a cloning site for a transgene; (iii) a posttranscriptional response element (e.g. the woodchuck hepatitis virus posttranscriptional regulatory element (WPRE)); and (iv) a poly-adenylation signal (e.g. from bovine growth hormone gene (BGHpA). Unique restriction endonuclease recognition sites (R1-R6) (shown in FIG. 1A and FIG. 1B) are also introduced between each component to facilitate the introduction of new genetic components into the specific sites in the construct. R3 (PmeI) 5′-GTTTAAAC-3′ and R4 (Pad) 5′-TTAATTAA-3′ enzyme sites are engineered into the cloning site to introduce an open reading frame of a transgene. These sequences are cloned into a pFastBac HT B plasmid obtained from ThermoFisher Scientific.

Production of ceDNA-Bacmids:

DH10Bac competent cells (MAX EFFICIENCY® DH10Bac™ Competent Cells, Thermo Fisher) are transformed with either test or control plasmids following a protocol according to the manufacturer's instructions. Recombination between the plasmid and a baculovirus shuttle vector in the DH10Bac cells are induced to generate recombinant ceDNA-bacmids. The recombinant bacmids are selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene are picked and cultured in 10 mL of media.

The recombinant ceDNA-bacmids are isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) is removed from the cells, filtered through a 0.45 μm filter, separating the infectious baculovirus particles from cells or cell debris.

Optionally, the first generation of the baculovirus (P0) is amplified by infecting naïve Sf9 or Sf21 insect cells in 50 to 500 ml of media. Cells are maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium are collected following centrifugation to remove cells and debris then filtration through a 0.45 μm filter.

The ceDNA-baculovirus comprising the test constructs are collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity is determined by the rate of cell diameter increase and cell cycle arrest, and change in cell viability every day for 4 to 5 days.

A “Rep-plasmid” as disclosed in FIG. 8A of PCT/US18/49996, which is incorporated herein in its entirety by reference, is produced in a pFASTBAC™-Dual expression vector (ThermoFisher) comprising both the Rep78 and Rep52 or Rep68 and Rep40. The Rep-plasmid is transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells are induced to generate recombinant bacmids (“Rep-bacmids”). The recombinant bacmids are selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies are picked and inoculated in 10 mL of selection media (kanamycin, gentamicin, tetracycline in LB broth). The recombinant bacmids (Rep-bacmids) are isolated from the E. coli and the Rep-bacmids are transfected into Sf9 or Sf21 insect cells to produce infectious baculovirus.

The Sf9 or Sf21 insect cells are cultured in 50 mL of media for 4 days, and infectious recombinant baculovirus (“Rep-baculovirus”) are isolated from the culture. Optionally, the first generation Rep-baculovirus (P0) are amplified by infecting naïve Sf9 or Sf21 insect cells and cultured in 50 to 500 ml of media. Between 3 and 8 days post-infection, the P1 baculovirus particles in the medium are collected either by separating cells by centrifugation or filtration or another fractionation process. The Rep-baculovirus are collected and the infectious activity of the baculovirus was determined. Specifically, four×20 mL Sf9 cell cultures at 2.5×10⁶ cells/mL are treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

With reference to FIG. 4B, Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm.

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the DNA obtained from co-infected Sf9 cells (as described herein) with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4D and 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4D).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g., GE HEALTHCARE ILUSTRA™ MICROSPIN™ G-25 columns are some art-known options for the endonuclease digestion. The assay includes for example, i) digest DNA with appropriate restriction endonuclease(s), ii) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g. Thermo Fisher, SYBR® Gold Nucleic Acid Gel Stain (10,000× Concentrate in DMSO) and epifluorescent light (blue) or UV (312 nm).

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

For comparative purposes, Example 1 describes the production of ceDNA vectors using an insect cell-based method and a polynucleotide construct template, and is also described in Example 1 of PCT/US18/49996, which is incorporated herein in its entirety by reference. For example, a polynucleotide construct template used for generating the ceDNA vectors of the present invention according to Example 1 can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 mL of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 mL of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 mL of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

A “Rep-plasmid” was produced in a pFASTBAC™-Dual expression vector (ThermoFisher) comprising both the Rep78 or Rep68 and Rep52 or Rep40. The Rep-plasmid was transformed into the DH10Bac competent cells (MAX EFFICIENCY® DH10Bac™ Competent Cells (Thermo Fisher)) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 mL of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 mL of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

### Example 2: Synthetic ceDNA Production Via Excision from a Double-Stranded DNA Molecule

Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIG. 6 in International patent application PCT/US2018/064242, filed Dec. 6, 2018).

In some embodiments, a construct to make a ceDNA vector comprises a regulatory switch as described herein.

For illustrative purposes, Example 1 describes producing ceDNA vectors as exemplary closed-ended DNA vectors generated using this method. However, while ceDNA vectors are exemplified in this Example to illustrate in vitro synthetic production methods to generate a closed-ended DNA vector by excision of a double-stranded polynucleotide comprising the ITRs and expression cassette (e.g., heterologous nucleic acid sequence) followed by ligation of the free 3′ and 5′ ends as described herein, one of ordinary skill in the art is aware that one can, as illustrated above, modify the double stranded DNA polynucleotide molecule such that any desired closed-ended DNA vector is generated, including but not limited to, ministring DNA, Doggybone™ DNA, dumbbell DNA and the like. Exemplary ceDNA vectors for production of transgenes and therapeutic proteins can be produced by the synthetic production method described in Example 2.

The method involves (i) excising a sequence encoding the expression cassette from a double-stranded DNA construct and (ii) forming hairpin structures at one or more of the ITRs and (iii) joining the free 5′ and 3′ ends by ligation, e.g., by T4 DNA ligase.

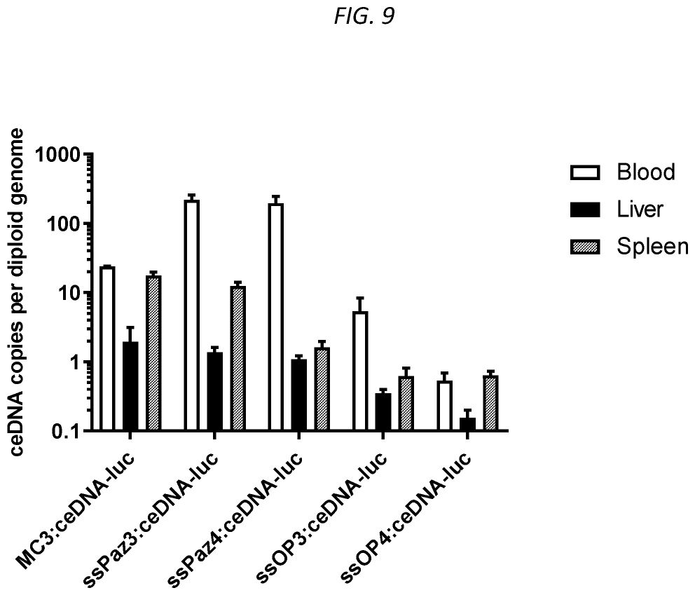

The double-stranded DNA construct comprises, in 5′ to 3′ order: a first restriction endonuclease site; an upstream ITR; an expression cassette; a downstream ITR; and a second restriction endonuclease site. The double-stranded DNA construct is then contacted with one or more restriction endonucleases to generate double-stranded breaks at both of the restriction endonuclease sites. One endonuclease can target both sites, or each site can be targeted by a different endonuclease as long as the restriction sites are not present in the ceDNA vector template. This excises the sequence between the restriction endonuclease sites from the rest of the double-stranded DNA construct (see FIG. 9 of PCT/US19/14122). Upon ligation a closed-ended DNA vector is formed.

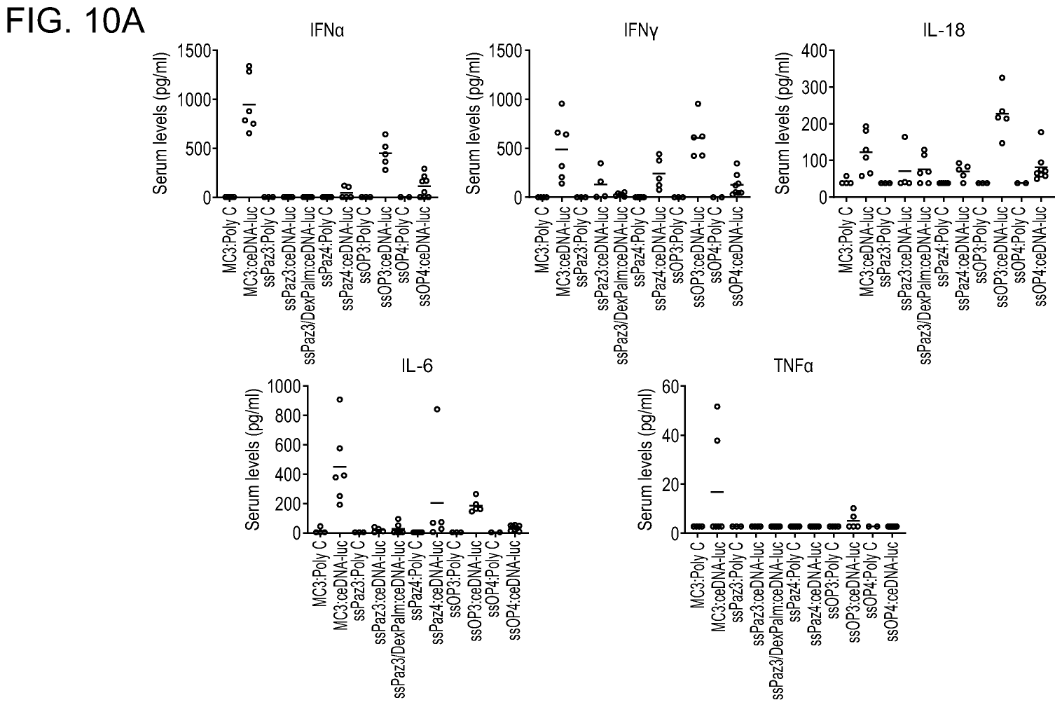

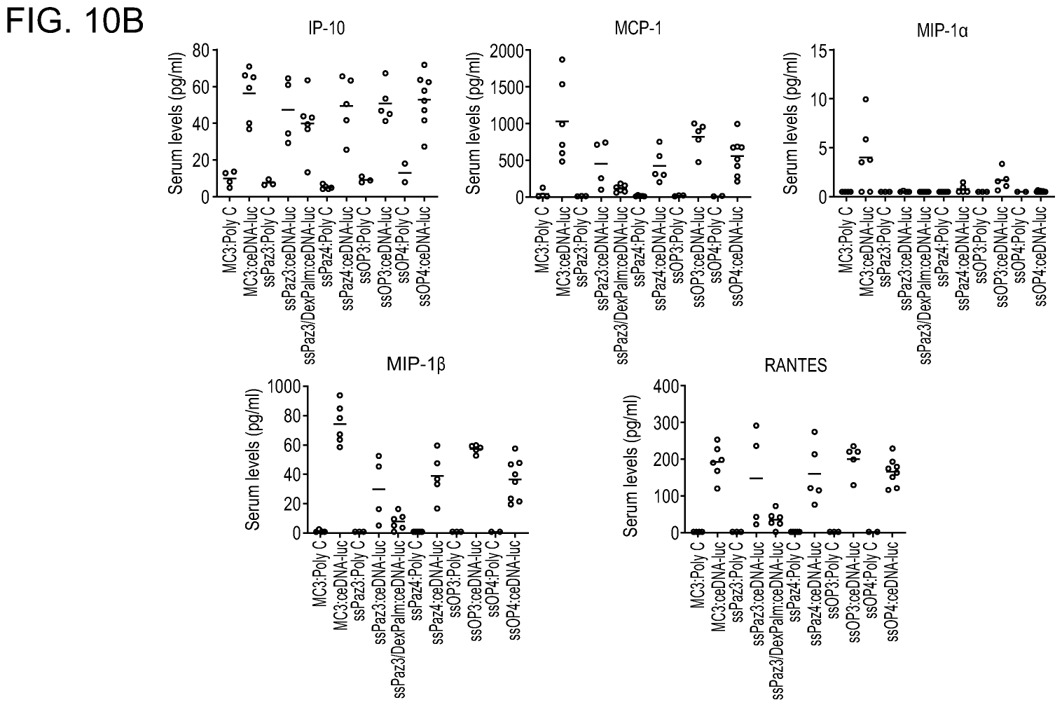

One or both of the ITRs used in the method may be wild-type ITRs. Modified ITRs may also be used, where the modification can include deletion, insertion, or substitution of one or more nucleotides from the wild-type ITR in the sequences forming B and B′ arm and/or C and C′ arm (see, e.g., FIGS. 6-8 and 10 FIG. 11B of PCT/US19/14122), and may have two or more hairpin loops (see, e.g., FIGS. 6-8 FIG. 11B of PCT/US19/14122) or a single hairpin loop (see, e.g., FIG. 10A-10B FIG. 11B of PCT/US19/14122). The hairpin loop modified ITR can be generated by genetic modification of an existing oligo or by de novo biological and/or chemical synthesis.

### Example 3: ceDNA Production Via Oligonucleotide Construction

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122 shows an exemplary method of ligating a 5′ ITR oligonucleotide and a 3′ ITR oligonucleotide to a double stranded polynucleotide comprising an expression cassette.

The ITR oligonucleotides can comprise WT-ITRs (see, e.g., FIGS. 6A, 6B, 7A and 7B of PCT/US19/14122, which is incorporated herein in its entirety). Exemplary ITR oligonucleotides include, but are not limited to those described in Table 7 in of PCT/US19/14122. Modified ITRs can include deletion, insertion, or substitution of one or more nucleotides from the wild-type ITR in the sequences forming B and B′ arm and/or C and C′ arm. ITR oligonucleotides, comprising WT-ITRs or mod-ITRs as described herein, to be used in the cell-free synthesis, can be generated by genetic modification or biological and/or chemical synthesis. As discussed herein, the ITR oligonucleotides in Examples 2 and 3 can comprise WT-ITRs, or modified ITRs (mod-ITRs) in symmetrical or asymmetrical configurations, as discussed herein.

### Example 4: ceDNA Production Via a Single-Stranded DNA Molecule

Another exemplary method of producing a ceDNA vector using a synthetic method is provided in Example 4 of PCT/US19/14122, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

An exemplary single-stranded DNA molecule for production of a ceDNA vector comprises, from 5′ to 3′: a sense first ITR; a sense expression cassette sequence; a sense second ITR; an antisense second ITR; an antisense expression cassette sequence; and an antisense first ITR.

A single-stranded DNA molecule for use in the exemplary method of Example 4 can be formed by any DNA synthesis methodology described herein, e.g., in vitro DNA synthesis, or provided by cleaving a DNA construct (e.g., a plasmid) with nucleases and melting the resulting dsDNA fragments to provide ssDNA fragments.

Annealing can be accomplished by lowering the temperature below the calculated melting temperatures of the sense and antisense sequence pairs. The melting temperature is dependent upon the specific nucleotide base content and the characteristics of the solution being used, e.g., the salt concentration. Melting temperatures for any given sequence and solution combination are readily calculated by one of ordinary skill in the art.

The free 5′ and 3′ ends of the annealed molecule can be ligated to each other, or ligated to a hairpin molecule to form the ceDNA vector. Suitable exemplary ligation methodologies and hairpin molecules are described in Examples 2 and 3.

### Example 5: Purifying and/or Confirming Production of ceDNA

Any of the DNA vector products produced by the methods described herein, e.g., including the insect cell based production methods described in Example 1, or synthetic production methods described in Examples 2-4 can be purified, e.g., to remove impurities, unused components, or byproducts using methods commonly known by a skilled artisan; and/or can be analyzed to confirm that DNA vector produced, (in this instance, a ceDNA vector) is the desired molecule. An exemplary method for purification of the DNA vector, e.g., ceDNA is using Qiagen Midi Plus purification protocol (Qiagen) and/or by gel purification,

The following is an exemplary method for confirming the identity of ceDNA vectors. ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors are further analyzed by digesting the purified DNA with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIG. 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a ceDNA vector with the continuous structure is expected to produce 2 kb and 4 kb fragments.

Therefore, to demonstrate in a qualitative fashion that isolated ceDNA vectors are covalently closed-ended as is required by definition, the samples are digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4E).

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard.

### Example 6: Preparation of Lipid Nanoparticle Formulations

ceDNA lipid nanoparticle (LNP) formulations comprising ss-OP were prepared as follows. Briefly, rapid mixing of two phases was carried out to form the intermediate LNP, where the ceDNA solution and lipid solution were mixed on NanoAssemblr at 3:1 flow rate ratio with total flow rate of 12 mL/min. The intermediate LNP was diluted with 1-3 vol of DPBS to decrease the ethanol concentration to stabilize the intermediate LNP. Ethanol was then removed and external buffer was replaced with DPBS by dialysis overnight at 4° C., either in a dialysis tube or float-lyzers (for small scale). Next, a concentration step was performed. The intermediate LNP was concentrated with Amicon Ultra-15 (10 KD MWCO) tube at 2000×g 4° C. for 20 minutes, three times. Finally, the LNP was filtered through a 0.2 μm pore sterile filter. The particle size of LNP can be determined by quasi-elastic light scattering using a Malvern Zetasizer Nano ZS (Malvern, UK) and the ceDNA encapsulation can be measured by Quant-iT PicoGreen dsDNA Assay Kit (Thermo Fisher Scientific).

Lipid nanoparticles (LNP) were prepared at a total lipid to ceDNA weight ratio of approximately 10:1 to 60:1. Preferably, LNPs were prepared at a total lipid to ceDNA weight ratio of 15:1 to 40:1. Briefly, a condensing agent (e.g., a cationic lipid such ss-OP or ss-Paz), a non-cationic-lipid (e.g., DSPC, DOPE, or DOPC), a component to provide membrane integrity (such as a sterol, e.g., cholesterol) and a conjugated lipid molecule (such as a PEG-lipid, e.g., 1-(monomethoxy-polyethyleneglycol)-2,3-dimyristoylglycerol, with an average PEG molecular weight of 2000 (“PEG₂₀₀₀-DMG”)), were solubilized in alcohol (e.g., ethanol) at a predetermined molar ratio (e.g., approximately 51:7:40:2±1 for each component). In certain examples, LNP were prepared without any non-cationic-lipid (e.g., DSPC, DOPE, or DOPC), and referred to as, for example, “ss-Paz3” or “ss-OP3” as they contain three different lipid components (as shown Table 1, LNP Nos. 3 and 5). LNP Nos. 6-19 are variants of ss-OP4 wherein LNP No. 6 was used in the animal studies designated as “ss-OP4” in FIGS. 7-18.

The ceDNA was diluted to a desired concentration in a buffer solution (1× Dulbecco's phosphate-buffered saline, DPBS). For example, the ceDNA was diluted to a concentration of 0.1 mg/mL to 0.25 mg/mL in a buffer solution comprising sodium acetate, sodium acetate and magnesium chloride, citrate, malic acid, or malic acid and sodium chloride. In one example, the ceDNA was diluted to 0.2 mg/mL in 10 to 50 mM citrate buffer, pH 4.0. The alcoholic lipid solution was mixed with ceDNA aqueous solution using, for example, syringe pumps or an impinging jet mixer, at a ratio of about 1:5 to 1:3 (vol/vol) with total flow rates above 10 mL/min. In some examples, the alcoholic lipid solution was mixed with ceDNA aqueous at a ratio of about 1:3 (vol/vol) with a flow rate of 12 mL/min. The alcohol was removed and the buffer was replaced with PBS by dialysis. Alternatively, the buffer was replaced with DPBS using centrifugal tubes. Alcohol removal and simultaneous buffer exchange was accomplished by, for example, dialysis or tangential flow filtration. The obtained lipid nanoparticles were filtered through a 0.2 μm pore sterile filter.

In one study lipid nanoparticles comprising exemplary ceDNAs were prepared using a lipid solution comprising ss-OP (Formula I), DOPC, cholesterol and DMG-PEG₂₀₀₀ (mol ratio of 51:7:40:2, ±1 for each component) or MC3, DSPC, Cholesterol and DMG-PEG₂₀₀₀ (mol ratio of 50:10:38.5:1.5). Aqueous solutions of ceDNA in buffered solutions were prepared. The lipid solution and the ceDNA solution were mixed using NanoAssembler at a total flow rate of 12 mL/min at a lipid to ceDNA ratio of 3:2 (vol/vol). Table 1 shows exemplary LNPs prepared in this study.

TABLE 1
Exemplary LNPs

| | | | Lipid | |
|---|---|---|---|---|
| LNP | | | Feed | |
| No. | Lipid mix* | Lipid Molar Ratio | [mg/mL] | ceDNA |
| 1 | ss-EC:Chol:DMG-PEG₂₀₀₀ | 68.0:29.1:2.9 | 2.6 | ceDNA- |
| | | | | luciferase |
| 2 | ss-EC:DOPC:Chol: DMG-PEG₂₀₀₀ | 51.0:7.3:38.8:2.9 | 2.6 | ceDNA- |
| | | | | luciferase |
| 3 | ss-Paz:Chol:DMG-PEG₂₀₀₀ | 68.0:29.1:2.9 | 2.6 | ceDNA- |
| | | | | luciferase |
| 4 | ss-Paz:DOPC:Chol:DMG-PEG₂₀₀₀ | 51.0:7.3:38.8:2.9 | 2.6 | ceDNA- |
| | | | | luciferase |
| 5 | ss-OP:Chol:DMG-PEG₂₀₀₀ | 68.0:29.1:2.9 | 2.6 | ceDNA- |
| | | | | luciferase |
| 6 | ss-OP:DOPC:Chol: DMG-PEG₂₀₀₀ | 51.0:7.3:38.8:2.9 | 2.6 | ceDNA- |
| | | | | luciferase |
| 7 | ss-OP:DOPC:Chol: DMG-PEG₂₀₀₀ | 50:10:38.5:1.5 | 2.6 | ceDNA- |
| | | | | luciferase |
| 8 | ss-OP:DOPE:Chol:DMG-PEG₂₀₀₀ | 50:10:38.5:1.5 | 2.6 | ceDNA- |
| | | | | luciferase |
| 9 | ss-OP:DOPC:Chol: DMG-PEG₂₀₀₀ | 51.7:7.4:39.4:1.5 | 2.6 | ceDNA- |
| | | | | luciferase |
| 10 | ss-OP:DSPC:Chol:DMG-PEG₂₀₀₀ | 51.0:7.3:38.8:2.9 | 2.6 | ceDNA- |
| | | | | luciferase |
| 11 | ss-OP:DSPC:Chol:DMG-PEG₂₀₀₀ | 51.7:7.4:39.4:1.5 | 2.6 | ceDNA- |
| | | | | luciferase |
| 12 | ss-OP:DOPE:Chol:DMG-PEG₂₀₀₀ | 51.0:7.3:38.8:2.9 | 2.6 | ceDNA- |
| | | | | luciferase |
| 13 | ss-OP:DOPE:Chol:DMG-PEG₂₀₀₀ | 51.7:7.4:39.4:1.5 | 2.6 | ceDNA- |
| | | | | luciferase |
| 14 | ss-OP:DOPC:Chol:DMG-PEG₂₀₀₀ | 47.5:10.0:40.7:1.8 | 2.6 | ceDNA- |
| | | | | luciferase |
| 15 | ss-OP:DSPC:Chol:DMG-PEG₂₀₀₀ | 47.5:10.0:40.7:1.8 | 2.6 | ceDNA- |
| | | | | luciferase |
| 16 | ss-OP:DOPE:Chol:DMG-PEG₂₀₀₀ | 47.5:10.0:40.7:1.8 | 2.6 | ceDNA- |
| | | | | luciferase |
| 17 | ss-OP:DOPE:Chol:C₁₈-PEG₂₀₀₀ | 51.7:7.4:39.4:1.5 | 2.6 | ceDNA- |
| | | | | luciferase |
| 18 | ss-OP:DOPE:Chol:C₁₈-PEG₂₀₀₀ | 51.0:7.3:38.8:2.9 | 2.6 | ceDNA- |
| | | | | luciferase |
| 19 | ss-OP:DOPE:Chol:C₁₈-PEG₂₀₀₀ | 50.0:7.1:38.1:4.8 | 2.6 | ceDNA- |
| | | | | luciferase |
| 20 | ss-OP:MC3:DOPE:Chol:DMG-PEG₂₀₀₀ | 25.5:25.5:7.3:38.8:2.9 | 2.6 | ceDNA- |
| | | | | luciferase |
| 21 | ss-OP:MC3:DOPE:Chol:DMG-PEG₂₀₀₀ | 34.0:17.0:7.3:38.8:2.9 | 2.6 | ceDNA- |
| | | | | luciferase |
| 22 | ss-OP:MC3:DOPE:Chol:DMG-PEG₂₀₀₀ | 40.8:10.2:7.3:38.8:2.9 | 2.6 | ceDNA- |
| | | | | luciferase |
| 23 | ss-OP:MC3:DOPE:Chol:DMG-PEG₂₀₀₀ | 45.9:5.1:7.3:38.8:2.9 | 2.6 | ceDNA- |
| | | | | luciferase |
| 24 | ss-OP:MC3:DOPE:Chol:DMG-PEG₂₀₀₀ | 48.4:2.5:7.3:38.8:2.9 | 2.6 | ceDNA- |
| | | | | luciferase |

*DOPC = dioleoylphosphatidylcholine; DOPE = dioleoylphosphatidylethanolamine; DSPC = distearoylphosphatidylcholine; MC3 = heptatriaconta-6,9,28,31-tetraen-19-yl-4- (dimethylamino)butanoate; Chol = Cholesterol; PEG = 1-(monomethoxy- polyethyleneglycol)-2,3-dimyristoylglycerol (DMG-PEG₂₀₀₀); ss-OP = COATSOME ® ss-OP and ss-EC = COATSOME ® ss-33/4PE-15.

### Analysis of Lipid Particle Formulations

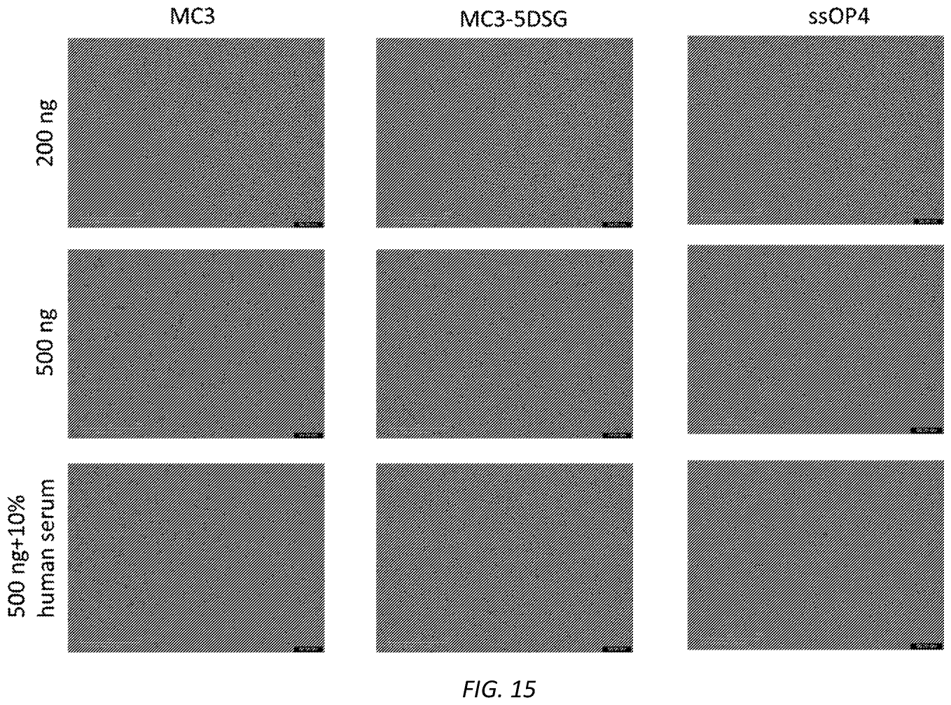

Lipid nanoparticle size and zeta potential, and encapsulation of ceDNA into the lipid nanoparticles were determined. Particle size was determined by dynamic light scattering and zeta potential was measured by electrophoretic light scattering (Zetasizer Nano ZS, Malvern Instruments). Results are shown in FIGS. 15-17.

Encapsulation of ceDNA in lipid particles was determined by Oligreen® (Invitrogen Corporation; Carlsbad, Calif.) or PicoGreen® (Thermo Scientific) kit. Oligreen® or PicoGreen® is an ultra-sensitive fluorescent nucleic acid stain for quantitating oligonucleotides and single-stranded DNA or RNA in solution. Briefly, encapsulation was determined by performing a membrane-impermeable fluorescent dye exclusion assay. The dye was added to the lipid particle formulation. Fluorescence intensity was measured and compared to the fluorescence observed upon addition of a small amount of nonionic detergent. Detergent-mediated disruption of the lipid bilayer releases the encapsulated ceDNA, allowing it to interact with the membrane-impermeable dye. Encapsulation of ceDNA was calculated as E=(I₀−I)/I₀, where I₀ refers to the fluorescence intensities with the addition of detergent and I refers to the fluorescence intensities without the addition of detergent.

Next, release of ceDNA from LNPs were determined. Endosome mimicking anionic liposome was prepared by mixing DOPS:DOPC:DOPE (mol ratio 1:1:2) in chloroform, followed by solvent evaporation at vacuum. The dried lipid film was resuspended in DPBS with brief sonication, followed by filtration through 0.45 μm syringe filer to form anionic liposome.

Serum was added to LNP solution at 1:1 (vol/vol) and incubated at 37° C. for 20 min. The mixture was then incubated with anionic liposome at desired anionic/cationic lipid mole ratio in DPBS at either pH 7.4 or 6.0 at 37° C. for another 15 min. Free ceDNA at pH 7.4 or pH 6.0 was calculated by determining unencapsulated ceDNA content by measuring the fluorescence upon the addition of PicoGreen (Thermo Scientific) to the LNP slurry (C_{free}) and comparing this value to the total ceDNA content that was obtained upon lysis of the LNPs by 1% Triton X-100 (Cₜₒₜₐₗ) where % free=C_{free}/Cₜₒₜₐₗ×100. The % ceDNA released after incubation with anionic liposome was calculated based on the equation below:

% ceDNA released=% free ceDNA_{mixed with anionic liposome}−% free ceDNA_{mixed with DPBS}

The pKa of formulated cationic lipids can be correlated with the effectiveness of the LNPs for delivery of nucleic acids (see Jayaraman et al., Angewandte Chemie, International Edition (2012), 51(34), 8529-8533; Semple et al., Nature Biotechnology 28, 172-176 (2010), both of which were incorporated by reference in their entirety). The preferred range of pKa was ˜5 to ˜7. The pKa of each cationic lipid was determined in lipid nanoparticles using an assay based on fluorescence of 2-(p-toluidino)-6-napthalene sulfonic acid (TNS). Lipid nanoparticles comprising of cationic lipid/DOPC/cholesterol/PEG-lipid (50/10/38.5/1.5 mol %) in DPBS at a concentration of 0.4 mM total lipid can be prepared using the in-line process as described herein and elsewhere. TNS can be prepared as a 100 μM stock solution in distilled water. Vesicles can be diluted to 24 μM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 μM and following vortex mixing fluorescence intensity was measured at room temperature in a SLM Aminco Series 2 Luminescence Spectrophotometer using excitation and emission wavelengths of 321 nm and 445 nm. A sigmoidal best fit analysis can be applied to the fluorescence data and the pKa was measured as the pH giving rise to half-maximal fluorescence intensity.

Binding of the lipid nanoparticles to ApoE were determined as follows. LNP (10 μg/mL of ceDNA) was incubated at 37° C. for 20 min with equal volume of recombinant ApoE3 (500 μg/mL) in DPBS. After incubation, LNP samples were diluted 10-fold using DPBS and analyzed by heparin sepharose chromatography on AKTA pure 150 (GE Healthcare) according to the conditions below:

HiTrap chromatographic conditions

| | |
|---|---|
| Column | HiTrap Heparin Sepharose HP 1 mL |

Equilibration buffer DPBS

| Wash buffer | DPBS | | |
|---|---|---|---|
| Elution buffer | 1M NaCl in 10 mM sodium phosphate | | |
| | buffer, pH 7.0 | | |
| Flow rate | 1 mL/min | | |
| Injection volume | 500 μL | | |
| Detection | 260 nm | | |
| | CV | A (%) | B (%) |
| Equilibration | 1 | 100 | 0 |
| Column wash | 4 | 100 | 0 |
| Elution (linear) | 10 | 0 | 100 |
| Equilibration | 3 | 100 | 0 |

### In Vitro Expression

Expression of ceDNA encapsulated into the lipid nanoparticles was assayed as follows. HEK293 cells were maintained at 37° C. with 5% CO₂ in DMEM+GlutaMAX™ culture medium (Thermo Scientific) supplemented with 10% Fetal Bovine Serum and 1% Penicillin-Streptomycin. Cells were plated in 96-well plates at a density of 30,000 cells/well the day before transfection. Lipofectamine™ 3000 (Thermo Scientific) transfection reagent was used for transfecting 100 ng/well of control ceDNA-luc according to the manufacturer's protocol. The control ceDNA was diluted in Opti-MEM™ (Thermo Scientific) and P3000™ Reagent was added. Subsequently, Lipofectamine™ 3000 was diluted to a final concentration of 3% in Opti-MEM™. Diluted Lipofectamine™ 3000 was added to diluted ceDNA at a 1:1 ratio and incubated for 15 minutes at room temperature. Desired amount of ceDNA-lipid complex or LNP was then directly added to each well containing cells. The cells were incubated at 37° C. with 5% CO₂ for 72 hours.

### Example 7: Evaluation of LNP Formulations of ceDNA in CD-1 Mice

The following study was carried out to evaluate LNPs containing SS-cleavable lipids in mice. As described herein, SS-series lipids contain dual sensing motifs that can respond to the intracellular environment: tertiary amines respond to an acidic compartment (endosome/lysosome) for membrane destabilization, and a disulfide bond that can cleave in reductive environment (cytoplasm). Exemplary lipid nanoparticle formulations were prepared according to Example 6 and tested in vivo.

Briefly, ceDNA-luc was formulated in LNPs containing SS-cleavable lipids and MC3 as described above and dosed at 0.5 mg/kg IV into male CD-1 mice. In one LNP, dexamethasone palmitate was included and co-formulated with ceDNA-luc in the ss-Paz3 (ssPalmE-Paz4-C2; also known as SS-33/1PZ-21) LNPs. As mentioned above, the numbers 3 and 4, as in ss-OP3 and ss-OP4; or in ss-Paz3 and ss-Paz4, represent total lipid components in LNP formulation. For example, ss-OP3 LNP contains three different lipid components: ss-OP, cholesterol and PEG-DMG. Similarly, ss-OP4 LNP has four different lipid components: ss-OP, DOPC, cholesterol and PEG-DMG. Dexamethasone palmitate (DexPalm) is an anti-inflammatory agent that inhibits leukocytes and tissue macrophages, and reduces inflammatory response. Endpoints included body weight, cytokines, liver/spleen biodistribution (qPCR), and luciferase activity (IVIS). The study design is outlined below in Table 2.

TABLE 2

| | Animals | | Dose | Dose | Treatment | |
|---|---|---|---|---|---|---|
| Group | per | | Level | Volume | Regimen, | Terminal Time |
| No. | Group | Test Material | (mg/kg) | (mL/kg) | ROA | Point |
| 1 | 6 | MC3:Poly C | 0.5 | 5 | Once on | N = 2 per group |
| 2 | 6 | MC3:ceDNA-luc | 0.5 | | by IV | on Day 0 |
| 3 | 6 | ss-Paz3:PolyC | 0.5 | | Day 0^{a} | N = 4 per group |
| 4 | 6 | ss-Paz3:ceDNA-luc | 0.5 | | | up to Day 28 |
| 5 | 6 | ss-Paz3:ceDNA-luc + | 0.5 | | | |
| | | DexPalm | | | | |
| 6 | 6 | ss-Paz4:PolyC | 0.5 | | | |
| 7 | 6 | ss-Paz4:ceDNA-luc | 0.5 | | | |
| 8 | 4 | ss-OP3:PolyC | 0.5 | | | Up to Day 28 |
| 9 | 6 | ss-OP3:ceDNA-luc | 0.5 | | | N = 2 per group |
| | | | | | | on Day 0 |
| | | | | | | N = 4 per group |
| | | | | | | up to Day 28 |
| 10 | 4 | ss-OP4:PolyC | 0.5 | | | Up to Day 28 |
| 11 | 6 | ss-OP4:ceDNA-luc | 0.5 | | | N = 2 per group |
| | | | | | | on Day 0 |
| | | | | | | N = 4 per group |
| | | | | | | up to Day 28 |
| 12 | 2 | MC3:ceDNA-luc | 0.5 | | | Day 1 |
| 13 | 2 | ss-OP4:ceDNA-luc | 0.5 | | | |

Housing: Group housed in clear polycarbonate cages with contact bedding on a ventilated rack in a procedure room.
Chow/Water: Mouse Diet 5058 and filtered tap water acidified with 1N HCl to a targeted pH of 2.5-3.0 were be provided to the animals ad libitum.
^{a}Animals may be enrolled in 2 cohorts (n = 2 and n = 4 as applicable per group) as needed for scheduling.
No. = Number;
IV = intravenous;
ROA = route of administration.
ss-PAZ (ssPalmE-Paz4-C2);
PolyC: polycytidylic acid

Blood samples were collected at interim time points, and at the end of the study (terminal) as outlined below.

TABLE 3
Blood Collection:

| | Sample Collection Times |
|---|---|
| Group | Whole Blood (Tail, saphenous or orbital) |
| Number | SERUM^{a} |
| 1-7, 9, 11 | Day 0 |
| 4 per group | 6 hours post Test Material dose (±5%) |
| 12 + 13 | Day 0 |
| | 6 hours post Test Material dose (±5%) |
| Volume/ | ~150 μL whole blood |
| Portion | |
| Processing/ | 1 aliquot frozen at nominally −70° C. |
| Storage | |

^{a}Whole blood was collected into serum separator tubes, with clot activator; MOV = maximum obtainable volume

TABLE 4
Blood Collection (Terminal)

| | |
|---|---|
| | Sample Collection Times |
| Group | Terminal |

| Number | SERUM^{a} | EDTA Whole Blood |
|---|---|---|
| 1-7, 9, and 11 | Day 0 | Day 0 |
| (2 per group) | 6 hours post Test | 6 hours post Test |
| | Material dose | Material dose |
| | (±5%) | (±5%) |
| 12 and 13 | | Day 1 |
| | | 24 hours post Test |
| | | Material dose |
| | | (±5%) |
| Portion | ½ MOV | ½ MOV or ~400 μL |
| Processing/ | 1 aliquot frozen at | 1 aliquot |
| Storage | nominally −70° C. | Store at 4° C. |

^{a}Whole blood was collected into serum separator tubes, with clot activator; MOV = maximum obtainable volume

Tissue was collected at the end of the study (terminal) as outlined below.

TABLE 5
Terminal Tissue Collection

| Group | Sample Collection Times | |
|---|---|---|
| Number | Liver | Spleen |
| 1-7, 9, and 11 | Day 0 | |
| (2 per group) | 5-6 hours post Test Material dose | |
| 12 and 13 | Day 1 | |
| | 24 hours post Test Material dose (±5%) | |
| Volume/ | Whole organ, weighed | Whole organ, weighed |
| Portion | Then divided | Then divided |
| Processing | Left liver lobe stored in | 4 × 15-25 mg pieces |
| | 10% NBF (EPL) | weighed and snap frozen |
| | 4 × 25-50 mg pieces | individually |
| | weighed snap frozen | |
| | individually | |

| | |
|---|---|
| Storage | Fixed samples stored refrigerated |
| | Frozen samples stored at nominally −70° C. |

No. = number, MOV = maximum obtainable volume; NBF = neutral buffered formalin; TBD = to be determined

The study details are set forth below. CD-1 mice of ˜4 weeks of age at arrival were obtained from Charles River (N=62). ceDNA containing a luciferase expression cassette was provided in lipid nanoparticles as described herein. Cage side observations were performed daily. Clinical observations were performed at ˜1 hour, ˜5-6 hours and ˜24 hours (remaining animals per group) post dose. Additional observations were be made per exception. Body weights for all animals were be recorded on Days 0, 1, 2, 3, 7, 14, 21 and 28 (prior to euthanasia). Additional body weights were recorded as needed. ceDNA were supplied in a concentration stock (0.5 mg/mL). Stock was warmed to room temperature and diluted with the provided PBS immediately prior to use. Prepared materials were stored at ˜4° C. if dosing is not performed immediately. The ceDNA for Groups 1-13 were dosed at 5 mL/kg on Day 0 by IV administration via lateral tail vein. Animals were enrolled in 2 or more cohorts as needed for scheduling. On Days 3, 7, 14 (optional Days 21 and 28), remaining animals in Groups 1-11 were dosed with luciferin at 150 mg/kg (60 mg/mL) via intraperitoneal (IP) injection at 2.5 mL/kg. ≤15 minutes post each luciferin administration Luminescence was obtained by using in vivo imaging system (IVIS) imaging as described below. Four (n=4) animals from each Group 1 through 7, 9 and 11, and two (n=2) animals from Groups 12 and 13 had interim blood collected on Day 0. After each collection animals received 0.5-1.0 mL lactated Ringer's, subcutaneously. Whole blood for serum was collected by tail-vein nick, saphenous vein or orbital sinus puncture (under inhalant isoflurane per facility SOPs). Whole blood was collected into a serum separator with clot activator tube and processed into one (1) aliquot of serum per facility SOPs. All samples were stored at nominally −70° C. until transferred or shipped on dry ice for analysis.

On Day 0, 5-6 hours post dose, for n=2 animals from each Group 1 through 7, 9 and 11 (not Groups 8 and 10) were euthanized by CO₂ asphyxiation followed by thoracotomy and exsanguination.

Maximum obtainable blood volume was collected by cardiac puncture, and divided: ½ collected into a serum separator with clot activator tube and processed into one (1) aliquot of serum per facility SOPs; ½ collected into EDTA coated tubes stored on 4° C. until shipped.

On Day 1, 24 hours post dose, for n=2 animals from each Groups 12 and 13 were euthanized by CO₂ asphyxiation followed by thoracotomy and exsanguination. Maximum obtainable blood volume was collected by cardiac puncture, and divided: ˜400 collected into EDTA coated tubes stored 4° C.; any remainder whole blood was discarded.

On Day 28, the remaining animals from each group (n=4) were euthanized by CO₂ asphyxiation followed by thoracotomy or cervical dislocation.

Following exsanguination, all animals underwent cardiac perfusion with saline. In brief, whole body intracardiac perfusion was performed by inserting 23/21-gauge needle attached to 10 mL syringe containing saline into the lumen of the left ventricle for perfusion. The right atrium was incised to provide a drainage outlet for perfusate. Gentle and steady pressure was applied to the plunger to perfuse the animal after the needle has been positioned in the heart. Adequate flow of the flushing solution was ensured until the exiting perfusate flows clear (free of visible blood) indicating that the flushing solution has saturated the body and the procedure is complete.

Terminal tissues were collected from moribund animals that were euthanized prior to their scheduled time point. Tissues were collected and stored from animals that were found dead, where possible. After euthanasia and perfusion, the liver and spleen were harvested and whole organ weights were recorded.

The left liver lobe was placed in histology cassettes and fixed in 10% neutral buffered, refrigerated (˜4° C.). Tissue in 10% NBF was kept refrigerated (˜4° C.) until shipped in sealed container on ice packs.

Out of the remaining liver, 4×˜25-50 mg sections (≤50 mg) were collected and weighed. Sections were snap frozen individually, stored at nominally −70° C. until shipped. All remaining liver was discarded.

From the spleen 4×˜15-25 mg sections (≤25 mg) were collected and weighed. Sections were snap frozen individually, stored at nominally −70° C. until shipped. All remaining spleen were be discarded.

Next, ceDNA expression was evaluated for Luciferase-04-sense in 10 mouse liver FFPE samples using RNAscope LS ISH assay, an in situ hybridization (ISH) assay method used to visualize single RNA molecules per cell in a sample.

10 mouse liver FFPE samples were provided in four treatment groups and one vehicle control, with 2 mice in each group). The following probes were used: Mm-PPIB (positive control); dapB (negative control); Luciferase-04-sense.

Positive and negative control assays were first be performed to assess tissue and RNA quality and to optimize assay conditions for the sample set, followed by performance of target assays on the samples that pass quality control (QC).

### In Vivo IVIS Imaging Protocol

In vivo imaging was carried out using the below materials and methods.

Materials:

Appropriate syringe for luciferin administration, appropriate device and/or syringe for luciferin administration, firefly Luciferin, PBS, pH meter or equivalent, 5-M NaOH, 5-M HCl, K/X anesthetics or Isoflurane.

### Procedure

### Luciferin Preparation:

Luciferin stock powder is stored at nominally −20° C.
Store formulated luciferin in 1 mL aliquots at 2-8° C. protect from light.
Formulated luciferin is stable for up to 3 weeks at 2-8° C., protected from light and stable for about 12 hrs at room temperature (RT).
Dissolve luciferin in PBS to a target concentration of 60 mg/mL at a sufficient volume and adjusted to pH=7.4 with 5-M NaOH (˜0.5 μl/mg luciferin) and HCl (˜0.5 μL/mg luciferin) as needed.
Prepare the appropriate amount according to protocol including at least a ˜50% overage.

### Injection and Imaging (Note: Up to 3 Animals May be Imaged at One Time)

Shave animal's hair coat (as needed).
Per protocol, inject 150 mg/kg of luciferin in PBS at 60 mg/mL via IP.
Imaging can be performed immediately or up to 15 minutes post dose.
Set isoflurane vaporizer to 1-3% (usually@2.5%) to anesthetize the animals during imaging sessions.
Isoflurane anesthesia for imaging session:
   Place the Animal into the isoflurane chamber and wait for the isoflurane to take effect, about 2-3 minutes.
   Ensure that the anesthesia level on the side of the IVIS machine is positioned to the “on” position.
   Place animal(s) into the IVIS machine and shut the door
Log into the IVIS computer and open the desired Acquisition Protocol. Recommended acquisition settings for highest sensitivity are: camera height at D level, F/Stop at fl, binning at medium resolution, and exposure time to auto.
Press the “ACQUIRE” in the camera control panel interface.
Insert labels onto all acquired images. Images are saved.

### Results

Minimal effects on body weight were observed in all dose groups of mice, as shown in FIG. 7. FIG. 8 is a graph that shows luciferase activity in each of the ceDNA LNP groups (MC3:PolyC; MC3:ceDNA-luc; ss-Paz3:PolyC; ss-Paz3: ceDNA-luc; ss-Paz3: ceDNA-luc+dexPalm; ss-Paz4:PolyC; ss-Paz4: ceDNA-luc; ss-OP3:PolyC; ss-OP3: ceDNA-luc; ss-OP4:PolyC; ss-OP4: ceDNA-luc). Luciferase expression in the ss-OP3: ceDNA-luc and ss-OP4: ceDNA-luc dose groups was similar to or superior to that of the MC3 dose group, but was not detectable in the ss-PAZ3: ceDNA-luc and ss-PAZ4: ceDNA-luc dose groups, as shown in FIG. 8. ceDNA was detected in the blood, liver and spleen by qPCR 6 h post administration in all dose groups, although the relative ratios varied, as shown in FIG. 9.

The effects of the SS-series lipids in the LNPs on cytokine and chemokine levels (pg/mL) in the serum of mice at 6 hours after dosing on day 0 are shown in FIG. 10A and FIG. 10B. Levels of interferon alpha (IFNα), interferon gamma (IFNγ), interleukin (IL)-18, IL-6, tumor necrosis factor alpha (TNFα), interferon gamma-induced protein 10 (IP-10; also known as CXCL10), monocyte chemoattractant protein-1 (MCP-1/CCL2), macrophage inflammatory proteins (MIP) 1α and MIP1β, and Regulated on Activation Normal T Cell Expressed and Secreted (RANTES) were determined. As shown in FIG. 10A and FIG. 10B, cytokine levels were significantly lower in the SS-series:ceDNA-luc dose groups as compared to the MC3:ceDNA-luc dose group, but still higher than the corresponding negative control PolyC dose groups. Dexamethasone palmitate (DexPalm) provided further reductions in some cytokines.

Compared to the MC3 group, the mice treated with the ss-OP4 LNPs had 100× fewer copies in the liver at 24 h (FIG. 9), while achieving equivalent or greater luciferase expression (FIG. 11) and lower cytokine releases (FIGS. 10A and 10B). Further, these studies also revealed the beneficial effects of dexamethasone palmitate in LNP formulation on cytokine responses when used in conjunction with ceDNA and ss-lipids.

Taken together, the results demonstrate that ss-OP4 outperformed MC3, where the ss-OP4 LNP formulations delivered fewer number of copies of ceDNA, while maintaining equivalent levels of ceDNA expression as compared to the MC3 LNP formulations. Further, the ss-OP4 LNPs exhibited significantly reduced cytokine releases as compared to the MC3 LNPs, indicating that the ceDNA-ss-OP4 LNPs had a positive impact on mitigating proinflammatory immune responses.

### Example 8: Evaluation of LNP Formulations of ceDNA in CD-1 Mice

The following study was carried out to evaluate LNPs containing SS-cleavable lipids used in conjunction with GalNAc in mice.

Exemplary lipid nanoparticle formulations were prepared according to Example 6 and tested in vivo. Briefly, ss-OP4 was prepared with ss-OP (Formula I), DOPC, cholesterol and DMG-PEG₂₀₀₀, and GalNAc with molar ratio of 50%:10%:38%:1.5%:0.5%, respectively. The study design is outlined below in Tables 6-7 below.

TABLE 6
Test Material Administration Cohort A

| | Animals | | Dose | Dose | | Terminal |
|---|---|---|---|---|---|---|
| Group | per | | Level | Volume | Treatment | Time |
| No. | Group | Treatment | (mg/kg) | (mL/kg) | Regimen | Point |
| 1 | 4 | PBS | NA | 5 | Once on | Day 21 |
| 2 | 4 | ss-OP4:ceDNA-luc | 0.5 | | Day 0, IV | |
| 3 | 4 | ss-OP4:ceDNA-luc | 2.0 | | | |
| 4 | 4 | ss-OP4/GalNAc:ceDNA-luc | 0.5 | | | |
| 5 | 4 | ss-OP4/GalNAc:ceDNA-luc | 2.0 | | | |

No. = Number;
IV = intravenous;
ROA = route of administration

TABLE 7
Test Material Administration Cohort B

| | Animals | | Dose | Dose | | Terminal |
|---|---|---|---|---|---|---|
| Group | per | | Level | Volume | Treatment | Time |
| No. | Group | Treatment | (mg/kg) | (mL/kg) | Regimen | Point |
| 1b | 2 | PBS | NA | 5 | Once on | Day 1 |
| 2b | 2 | ss-OP4:ceDNA-luc | 0.5 | | Day 0, IV | |
| 3b | 2 | ss-OP4:ceDNA-luc | 2.0 | | | |
| 4b | 2 | ss-OP4/GalNAc:ceDNA-luc | 0.5 | | | |
| 5b | 2 | ss-OP4/GalNAc:ceDNA-luc | 2.0 | | | |

No. = Number;
IV = intravenous;
ROA = route of administration

The study details are set forth below.

Species (number, sex, age): CD-1 mice (N=62 and 4 spare, male, ˜4 weeks of age at arrival) were obtained from Charles River Laboratories.

Class of Compound: ceDNA was provided in lipid nanoparticles as described herein.

Cage Side Observations: Cage side observations were performed daily.

Clinical Observations: Clinical observations were performed ˜1, ˜5-6 and ˜24 hours post the Day 0 Test Material dose. Additional observations were made per exception.

Body Weights: Body weights for all animals, as applicable, were recorded on Days 0, 1, 2, 3, 4, 7, 14 & 21 (prior to euthanasia). Additional body weights were recorded as needed.

Pre-Treatment & Test Material Dose Formulation: Pre-Treatment & Test articles were supplied in a concentration stock. Stock was warmed to room temperature and diluted with the provided PBS immediately prior to use. Prepared materials were stored at ˜4° C. if dosing is not performed immediately.

Dose Administration: Test articles were dosed at 5 mL/kg on Day 0 for Groups 1-5 by intravenous administration via lateral tail vein. Cohorts A and B may have different Day 0 dates.

In-life Imaging: On Days 4, 7, 14 & 21 animals in Groups 1-5, Cohort A only, were dosed with luciferin at 150 mg/kg (60 mg/mL) via intraperitoneal (IP) injection at 2.5 mL/kg. ≤15 minutes post each luciferin administration. Luminescence was obtained by using in vivo imaging system (IVIS) imaging.

Anesthesia Recovery: Animals were monitored continuously while under anesthesia, during recovery and until mobile.

Interim Blood Collection: All animals in Groups 1-5, Cohort A only, had interim blood collected on Day 0; 6 hours post Test Material dose (±5%). After collection animals received 0.5-1.0 mL lactated Ringer's; subcutaneously. Whole blood for serum was collected by tail-vein nick, saphenous vein or orbital sinus puncture (under inhalant isofluranes). Whole blood was collected into a serum separator with clot activator tube and processed into one (1) aliquot of serum. All samples were stored at nominally −70° C. until shipping for analysis.

Euthanasia & Terminal Collection: On Day 1, 24 hours post dose (±5%), for n=2 animals from each Group 1-7 Cohort B were euthanized by CO₂ asphyxiation followed by thoracotomy and exsanguination. Blood was placed into EDTA coated tubes and whole blood (processed or unprocessed) was stored refrigerated until shipped.

Perfusion: Following exsanguination, all animals underwent cardiac perfusion with saline. In brief, whole body intracardiac perfusion was performed by inserting 23/21-gauge needle attached to 10 mL syringe containing saline into the lumen of the left ventricle for perfusion. The right atrium was incised to provide a drainage outlet for perfusate. Gentle and steady pressure was applied to the plunger to perfuse the animal after the needle has been positioned in the heart. Adequate flow of the flushing solution was ensured until the exiting perfusate flows clear (free of visible blood) indicating that the flushing solution has saturated the body and the procedure is complete.

Tissue Collection: Terminal tissues were collected from moribund animals in Cohort B that were euthanized prior to their scheduled time point. If possible, tissues were collected and stored from animals that were found dead. After euthanasia and perfusion, the liver, spleen, kidney and both lungs were harvested and whole organ weights were recorded.

The left liver lobe was placed in histology cassettes and fixed in 10% neutral buffered, refrigerated (˜4° C.). Tissue in 10% NBF was kept refrigerated (˜4° C.) until shipped in sealed container on ice packs.

Out of the remaining liver, 4×˜25-50 mg sections (≤50 mg) were collected and weighed.

Sections were snap frozen individually, stored at nominally −70° C. until shipped. All remaining liver was discarded.

From the left kidney 4×˜15-25 mg sections (≤25 mg) and were collected and weighed. Sections were snap frozen individually, stored at nominally −70° C. until shipped. All remaining kidney was discarded.

From the spleen 4×˜15-25 mg sections (≤25 mg) and was collected and weighed. Sections were snap frozen individually, stored at nominally −70° C. until shipped. All remaining spleen was discarded.

From the lungs 4×˜15-25 mg sections (≤25 mg) (2 pieces from each lung) were collected and weighed. Sections were snap frozen individually, stored at nominally −70° C. until shipped. All remaining lung was discarded.

On Day 21, animals in Cohort A were euthanized by CO₂ asphyxiation followed by thoracotomy or cervical dislocation. No tissues were collected.

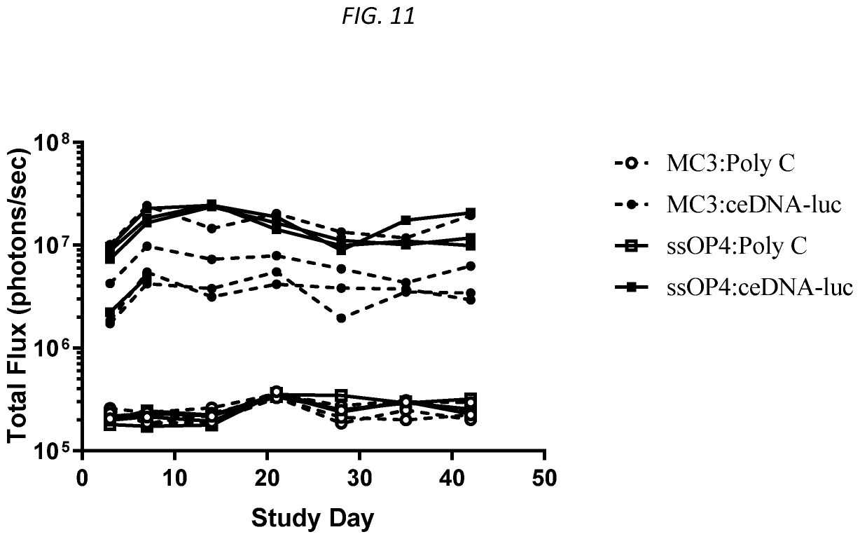

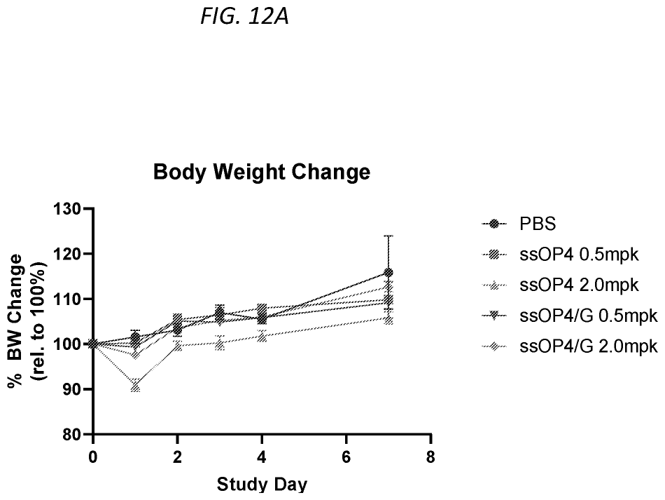

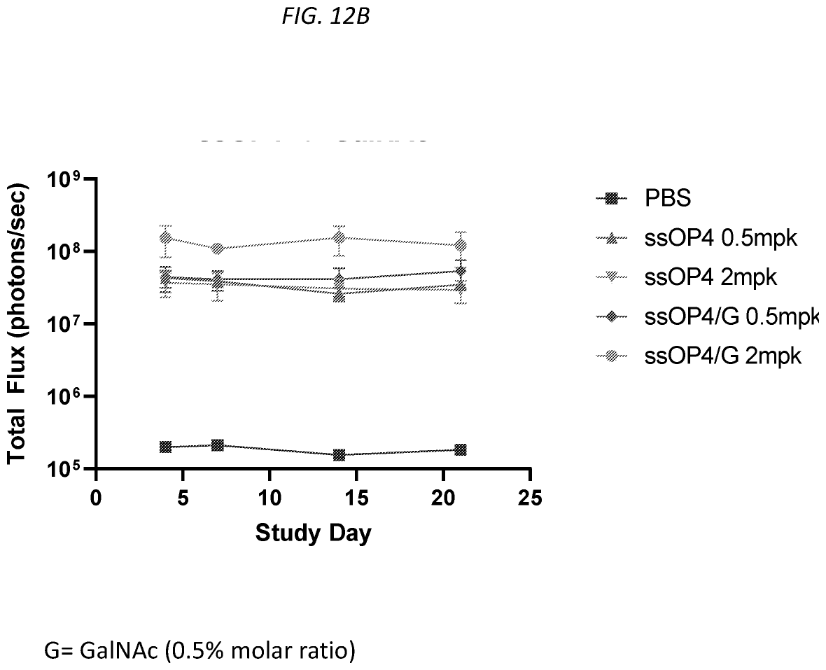

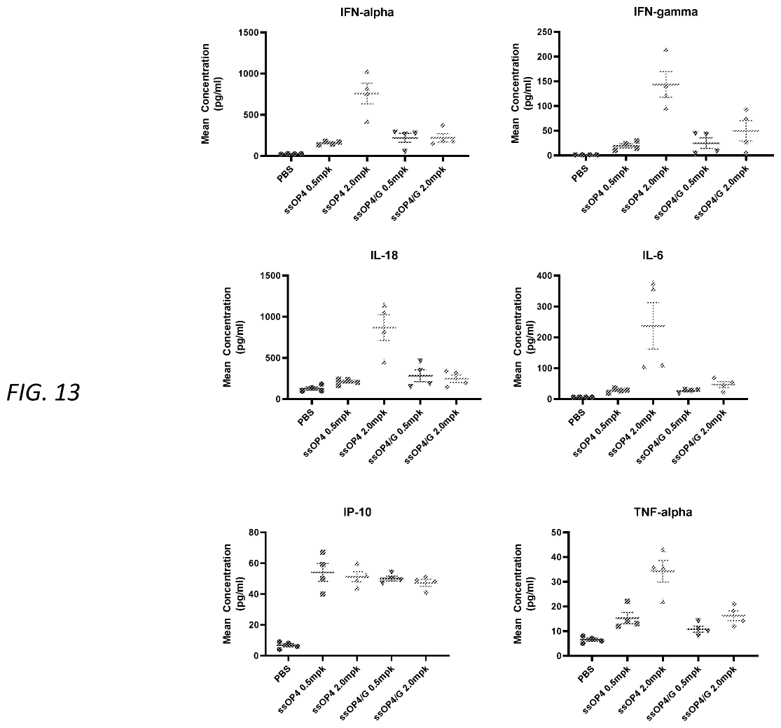

Results: The ss-OP4-ceDNA-treated mice (at doses of 0.5 and 2.0 mg/kg) demonstrated prolonged significant fluorescence, and hence luciferase transgene expression without exhibiting any adverse reaction. Throughout the study, mice continued to exhibit weight gain as shown in FIG. 12A. As shown in FIGS. 12B and 13, the presence of GalNAc (as in ss-OP4:G, 0.5% of GalNAc in molar ratio for total weight of LNP) in the ss-OP4-ceDNA formulation increased expression levels of ceDNA-luc while mitigating proinflammatory responses by reducing IFNα, IFNγ, IL-18, IL-6, IP-10 and/or TNF-α release. This data suggests that targeting the ceDNA formulated with ss-OP4 to specific tissues expressing GalNAc receptors (e.g., liver) improves targeting efficiency, which leads to enhancement of ceDNA expression while migrating inflammatory responses.

### Example 9: Evaluation of LNP Formulations of ceDNA in CD-1 Mice

The following study was carried out to evaluate LNPs containing SS-cleavable lipids in mice. As described herein, SS-series lipids contain dual sensing motifs that can respond to the intracellular environment: tertiary amines respond to an acidic compartment (endosome/lysosome) for membrane destabilization, and a disulfide bond that can cleave in reductive environment (cytoplasm). Exemplary lipid nanoparticle formulations were prepared according to Example 6 and tested in vivo.

The study design is outlined below in Table 8.

TABLE 8

| | | | | Dose | Treatment | Terminal |
|---|---|---|---|---|---|---|
| Group | #Animals/ | | Dose Level | Volume | Regimen, | Time |
| No. | Group | Test Material | (mg/kg) | (mL/kg) | ROA | Point |
| 1 | 4 | MC3:ceDNA-luc | 0.5 | 5 once | Once by IV on | Day 56 |
| 2 | 4 | ss-OP4 | 0.5 | by IV | Day 0 | |
| 3 | 4 | ss-OP4:ceDNA-luc | 0.1 | | | |
| 4 | 4 | ss-OP4:ceDNA-luc | 0.5 | | | |
| 5 | 4 | ss-OP4:ceDNA-luc + | 0.1 | | | |
| | | DexP | | | | |
| 6 | 4 | ss-OP4:ceDNA-luc + | 0.5 | | | |
| | | DexP | | | | |
| 7 | 4 | ss-OP4:ceDNA-luc + | 1.0 | | | |
| | | DexP | | | | |
| 8 | 4 | ss-OP4:ceDNA-luc + | 2.0 or | | Day 1 | |
| | | DexP | 0.75 | | | |

Test article storage (stock formulations): Test articles are supplied by the Sponsor in a concentrated stock (0.5 mg/mL) and stored at nominally 4° C. until use.
Residual test article is stored at nominally 4° C.
Housing: Group housed in clear polycarbonate cages with contact bedding on a ventilated rack in a procedure room.
Chow/Water: Mouse Diet 5058 and filtered tap water acidified with 1N HCl to a targeted pH of 2.5-3.0 will be provided to the animals ad libitum.
No. = Number;
IV = intravenous;
ROA = route of admilustratton.

Blood samples (including interim blood samples) were collected as outlined below in Tables 9 and 10.

TABLE 9

| | |
|---|---|
| | Sample Collection Times |
| Group | Whole Blood (Tail, saphenous or Orbital) |
| Number | SERUMS |

| | | |
|---|---|---|
| 1-7 | Day 0 | Day 1 |
| | 6 hours post Test | 24 hours post Test |
| | Material dose | Material dose (±5%) |
| 8 | Day 1 | Day 2 |
| | 6 hours post Test | 24 hours post Test |
| | Material dose | Material dose ±5%) |
| Volume/ | 150 ut whole blood | 50 μ1, whole blood |
| Portion | | (in vitro) |
| Processing/ | 1 aliquot frozen at | |
| Storage | nominally −70° C. | |

TABLE 10

| | Sample | DO/1 | Dl/2 |
|---|---|---|---|
| | Destination | (6 hr) | (24 hr) |
| Volume (mL) | Cytokine | 0.15 mL | |
| Whole Blood | ALT/AST | | 0.05 mL |
| | total/day (mL) | 0.15 mL | 0.05 mL |

The study details are set forth below.

Species (number, sex, age): CD-1 mice (N=62 and 4 spare, male, ˜4 weeks of age at arrival) were obtained from Charles River Laboratories.

Class of Compound: ceDNA was provided in lipid nanoparticles as described herein.

Cage Side Observations: Cage side observations were performed daily.

Clinical Observations: Clinical observations were performed ˜1, ˜5-6 and ˜24 hours post the Day 0 Test Material dose. Additional observations were made per exception.

Body Weights: Body weights for all animals, as applicable, were recorded on 0, 1, 2, 3, 4, 7, 14, 21, 28, 35, 42, 49 and 56 (prior to euthanasia). Additional body weights were recorded as needed.

Test Material Dose Formulation: Test articles were be supplied in a concentration stock (0.5 mg/mL). Stock was warmed to room temperature and diluted with the provided PBS immediately prior to use. Prepared materials were stored at −4° C. if dosing is not performed immediately.

Dose Administration: Test articles for Groups 1-7 were dosed at 5 mL/kg on Day 0 by IV administration via lateral tail vein. Test articles for Group 8 were dosed at 5 mL/kg on Day 1 by IV administration via lateral tail vein. The dose level of 2.0 mg/kg or 0.75 mg/kg was determined after the 6 and 24 hour clinical observations of Group 7. If any adverse effects are seen the lower dose was administered.

In-life Imaging: On days 7, 14, 21, 28, 35, 42, 49 and 56 animals in Groups 1-8 were dosed with luciferin at 150 mg/kg (60 mg/mL) via intraperitoneal (IP) injection at 2.5 mL/kg. ≤15 minutes post each luciferin administration. Luminescence was obtained by using in vivo imaging system (IVIS) imaging as described in Example 7.

Anesthesia Recovery: Animals were monitored continuously while under anesthesia, during recovery and until mobile.

Blood Collection: All animals had blood collected on Day 0 & 1 and Day 1 & 2 per table Sample Collection table above. After each collection animals received 0.5-1.0 mL lactated Ringer's, subcutaneously.

Whole blood for serum was collected by tail-vein nick, saphenous vein or orbital sinus puncture (under inhalant isoflurane per facility SOPs). Whole blood was collected into a serum separator with clot activator tube and processed into one (1) aliquot of serum per facility SOPs. All samples were stored at nominally −70° C.

Day 1/2 samples were analyzed by the Testing Facility for ALT/AST by ELISA.

Euthanasia: On Day 56, animals were euthanized by CO₂ asphyxiation followed by thoracotomy or cervical dislocation. No tissues were collected.

Report: A data report was issued for this study. Items included IVIS data, individual and group means (as applicable) for body weight, volume of TA administered per animal, times of dose administration, sample collections and euthanasia, clinical observations (as applicable) and mortality (as applicable).

Results: Minimal effects on body weight were observed in all dose groups of mice (data not shown). The ss-OP4 LNPs exhibited reduced cytokine releases as compared to the MC3 LNPs, indicating that the ceDNA-ss-OP4 LNPs had a positive impact on mitigating proinflammatory immune responses (data not shown). Dexamethasone palmitate (DexPalm) provided further reductions in some cytokines all groups tested with DexPalm. Luciferase expression in the ss-OP4: ceDNA-luc dose groups was similar to or superior to that of the MC3 dose group (data not shown).

### Example 10: Evaluation of ceDNA LNP Formulations by Route of Administration in Male CD-1 Mice

The following study was carried out to evaluate LNPs containing SS-cleavable lipids in mice, administered by intravenous (IV) or subcutaneous (SC) injection.

Briefly, ceDNA-luc was formulated in LNPs containing ss-OP4 cleavable lipids or MC3. As described above, ss-OP4 LNP has four different lipid components: ss-OP, DOPC, cholesterol and PEG-DMG. The formulations shown below in Table 11 were prepared and tested.

TABLE 11

| LNP | Composition | Molar Ratio | | | | |
|---|---|---|---|---|---|---|
| MC3 | MC3:DSPC:Chol:DMG-PEG2000 | 50.0 | 10.0 | 38.5 | 1.5 | |
| ss-OP4/G_1^{st} generation | SS-OP:DOPC:Chol:DMG- | 50.7 | 7.3 | 38.6 | 2.9 | 0.5 |
| | PEG2000:DSPE-PEG-GalNAc4 | | | | | |
| ss-OP4/G_N/P-10 + beta- | SS-OP:DOPC:β-sitosterol:DMG- | 50.7 | 7.3 | 38.6 | 2.9 | 0.5 |
| sito + Malic | PEG2000:DSPE-PEG-GalNAc4 | | | | | |

N/P-10 is the ratio of amino group from SS-OP to phospho group from ceDNA. β-sitosterol (sito) is a cholesterol analog. Malic acid is the buffer for ceDNA before mixing with lipid solution in ethanol. ss-OP4 is ss-OP4 figures and G represents GalNAc.

The study design is outlined below in Table 12.

TABLE 12

| Group | Animals | | Dose Level | Dose Volume | Treatment | Terminal |
|---|---|---|---|---|---|---|
| No. | per Group | Treatment | (mg/kg) | (mL/kg) | Regimen | Time Point |
| 1 | 6 | MC3 | 0.5 | 5 | Once on | Day 1* or 28 |
| 2 | 6 | ss-OP4/GalNAc_1st generation | | | Day 0, IV | |
| 3 | 6 | ss-OP4/GalNAc_N/P-10 + | | | | |
| | | beta-sito + Malic | | | | |
| 4 | 6 | ss-OP4/GalNAc_1st generation | | | Once on | |
| | | | | | Day 0, | |
| | | | | | slow IV | |
| 5 | 6 | Empty | | | Once on | |
| 6 | 6 | MC3 | | | Day 0, SC | |
| 7 | 6 | ss-OP4/GalNAc_1st generation | | | | |
| 8 | 6 | ss-OP4/GalNAc_N/P-10 + beta- | | | | |
| | | sito + Malic (2^{nd} generation) | | | | |

No. = Number;
IV = intravenous;
ROA = route of administration
*n = 2 per group at 24 hours post dose
ss-OP4 = ssOP4 (Figures)
G = GalNAc

Blood samples were collected as outlined below in Table 13 (interim blood collection) and Table 14 (terminal blood collection).

TABLE 13

| | Sample Collection Times |
|---|---|
| Group | Whole Blood (Tail, saphenous or orbital) |
| Number | SERUM^{a} |
| 1-8 | Day 0 |
| only n = 4 | 6 hours post Test Material dose (±5%) |
| per group | |
| Volume/ | ~150 μL whole blood |
| Portion | |
| Processing/ | 1 aliquot frozen at |
| Storage | nominally −70° C. |

^{a}Whole blood was collected into serum separator tubes, with clot activator

TABLE 14

| | Sample Collection Times |
|---|---|
| Group | Terminal |
| Number | WHOLE BLOOD |
| N = 2 per | Day 1 |
| group | 24 hours post Test Material dose (±5%) |
| N = 4 per | Day 28 |
| group | |
| Portion | MOV |
| Processing/ | EDTA |
| Storage | DO NOT PROCESS/DO NOT FREEZE |
| | 5° C. ± 3° C. |

MOV = maximum obtainable volume

Terminal tissue was collected as outlined below in Table 15.

TABLE 15

| | Sample Collection Times | | | | | |
|---|---|---|---|---|---|---|
| Group | | Kidneys | | Lung | Injection Site | Naïve Skin |
| Number | Liver | (both) | Spleen | (both) | Groups 5-8 | Groups 5-8 |
| N = 2 per | Day 1 | | | | | |
| group | 24 hours post Test Material dose (±5%) | | | | | |
| N = 4 per | Day 28 | | | | | |
| group | | | | | | |
| Volume/ | Whole organ, weighed | | | | Marked | Equal portion |
| Portion | Then divided | | | | section of | of dorsal rump |
| | | | | | intrascapular | skin, shaved |
| | | | | | skin, shaved | |
| Processing | Left liver | | ⅓ Spleen | | Placed flat in cassette with | |
| | lobe stored | | stored in | | sponge in 10% NBF (EPL) | |
| | in 10% | | 10% NBF | | | |
| | NBF | | (EPL) | | | |
| | (EPL) | | | | | |

| | |
|---|---|
| 4 × 25-50 mg pieces | 4 × 15-25 mg pieces |
| weighed snap | weighed and snap |
| frozen individually | frozen individually |
| (Lake Pharma) | (Lake Pharma) |

| | | |
|---|---|---|
| Storage | Fixed samples stored refrigerated | Fixed samples stored |
| | Frozen samples stored at nominally | refrigerated |
| | −70° C. | |

No. = number

The study details are set forth below.

Species (number, sex, age): CD-1 mice (N=48 and 4 spare, male, ˜4 weeks of age at arrival) were obtained from Charles River Laboratories.

Class of Compound: ceDNA was provided in lipid nanoparticles as described herein.

Cage Side Observations: Cage side observations were performed daily.

Clinical Observations: Clinical observations were performed ˜1, ˜5-6 and ˜24 hours post the Day 0 Test Material dose. Additional observations were made per exception.

Body Weights: Body weights for all animals, as applicable, were recorded on 0, 1, 2, 3, 4, 7, 14, 21, 28, 35, 42, 49 and 56 (prior to euthanasia). Additional body weights were recorded as needed.

Test Material Dose Formulation: Test articles were be supplied in a concentration stock (0.5 mg/mL). Stock was warmed to room temperature and diluted with the provided PBS immediately prior to use. Prepared materials were stored at −4° C. if dosing is not performed immediately.

Dose Administration IV: Test articles were dosed at 5 mL/kg on Day 0 for Groups 1-4 Groups 1-3 by intravenous BOLUS administration via lateral tail vein and Group 4 by SLOW administration by syringe pump, over 45 seconds; via lateral tail vein.

SC Injection Site Preparation: Prior to dose administration on Day 0, animals in Groups 5-8 were anesthetized with inhalant isoflurane to effect and the intrascapular region were shaved of fur. At least once a week the site was re-shaved, while the animals were being anesthetized for IVIS imaging.

Dose Administration SC: While anesthetized, test articles were dosed at 5 mL/kg on Day 0 for Groups 5-8 by subcutaneous administration in the intrascapular region.

With indelible ink, the skin will be marked around the area of injection material. The site will be remarked as needed until necropsy.

In-life Imaging: On Days 3, 7, 14, 21 & 28 remaining animals in Groups 1-8, were dosed with luciferin at 150 mg/kg (60 mg/mL) via intraperitoneal (IP) injection at 2.5 mL/kg. ≤15 minutes post each luciferin administration. Luminescence was obtained by using in vivo imaging system (IVIS) imaging as described in Example 7.

Anesthesia Recovery: Animals were monitored continuously while under anesthesia, during recovery and until mobile.

Blood Collection: Only 4 animals per group in Groups 1-8, had interim blood collected on Day 0; 6 hours post Test Material dose (±5%). After collection animals received 0.5-1.0 mL lactated Ringer's; subcutaneously.

Whole blood for serum was collected by tail-vein nick, saphenous vein or orbital sinus puncture (under inhalant isoflurane per facility SOPs). Whole blood was collected into a serum separator with clot activator tube and processed into one (1) aliquot of serum per facility SOPs. All samples were stored at nominally −70° C.

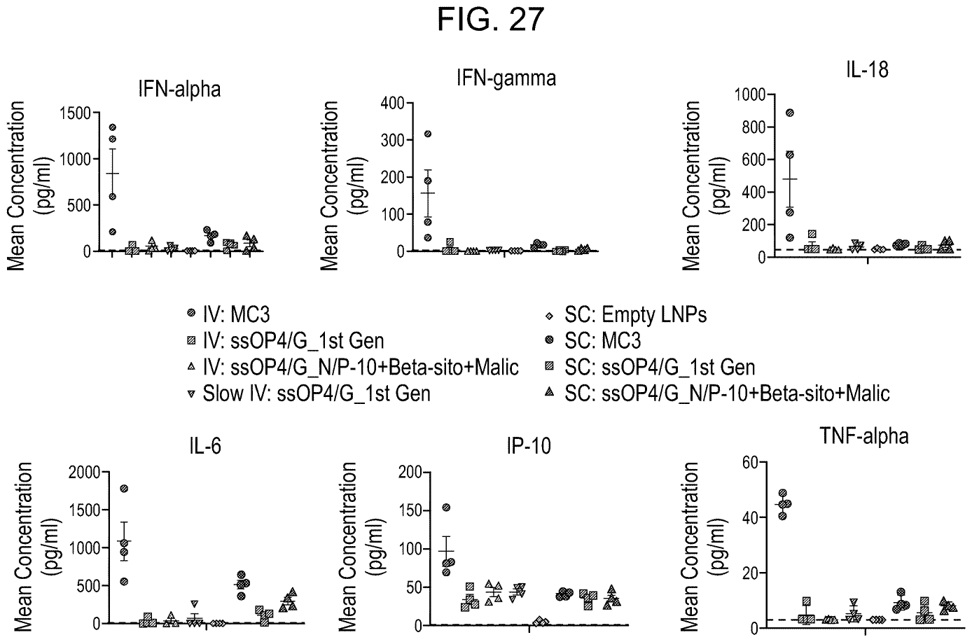

Results: As shown in FIG. 26, the mice treated with MC3 or ss-OP4-ceDNA administered intravenously (IV) demonstrated prolonged significant fluorescence, and hence luciferase transgene expression. Further, luciferase expression in the ss-OP4: ceDNA-luc IV dose groups was similar to or superior to that of the MC3 IV dose group. In comparison, the mice treated with MC3 or ss-OP4-ceDNA administered subcutaneously (SC) did not show significant fluorescence. Moreover, as shown in FIG. 27, The ss-OP4-ceDNA formulation administered either intravenously or subcutaneously mitigated proinflammatory responses by reducing IFNα, IFNγ, IL-18, IL-6, IP-10 and/or TNF-α release.

### Example 11: Evaluation of ceDNA LNP Formulations in Non-Human Primates

The following study was carried out to evaluate the tolerability of ceDNA LNPs containing SS-cleavable lipids used in conjunction with GalNAc after a 70-minute intravenous infusion to male cynomolgus monkeys. Exemplary lipid nanoparticle (LNP) formulations comprising ceDNA carrying Factor IX were prepared according to Example 6 and tested in vivo. LNP Formulation nos. 1 and 2 were standard non-cleavable cationic lipids. LNP Formulation #3 was ss-OP4+GalNac. As mentioned above, the number 4 in ss-OP4, represents total lipid components in the LNP formulation. For example, ss-OP4 LNP has four different lipid components: ss-OP, DOPC, cholesterol and PEG-DMG with a molar ratio of approximately 51:7:39:3, respectively, as in lipid nanoparticle no. 6 of Table 1.

All animals in all Groups were administered diphenhydramine and dexamethasone prior to the start of dosing. LNP Formulations #1, 2 or 3 were administered by IV infusion over an approximate 70-minute period. Endpoints included cytokine analysis, complement analysis, analysis of liver enzymes (AST, ALT), coagulation and anti-PEG IgG/IgM. The study design is outlined below in Table 16.

TABLE 16

| | No. | | | Dose Level | | Dose | | Biopsy and |
|---|---|---|---|---|---|---|---|---|
| Gr. | of | | | (mg/kg/ | Conc. | Volume | Dose Route/ | Sampling |
| No. | An. | Pre-Treatment | Test Material | dose) | (mg/mL) | (mL/kg) | Regimen | Time points |
| 1 | 1 | DPH and Dex | LNP Formulation #1 | 0.01 | 0.002 | 5 | 70 min IV | Blood Collection: |
| | | 30 minutes | non-cleavable LNP: | | | | infusion on | pre-dose, 15 |
| | | prior to dosing | ceDNA—human FIX | | | | day 0 | minutes, 6 and |
| 2 | 1 | | LNP Formulation #1 | 0.05 | 0.01 | | Infusion rate | 24-hours post |
| | | | non-cleavable LNP: | | | | for first 15 | end of infusion |
| | | | ceDNA—hFIX | | | | minutes: | |
| 3 | 1 | | LNP Formulation #1 | 0.1 | 0.02 | | 0.415 mL/kg | Liver & |
| | | | non-cleavable LNP: | | | | Infusion rate | Spleen Bx: |
| | | | ceDNA—hFIX | | | | for the | 24 hours |
| 4 | 1 | DPH and Dex | LNP Formulation #2 | 0.1 | 0.02 | | remaining 55 | Blood |
| | | 30 minutes | Non-cleavable LNP: | | | | minutes: | Collection: |
| | | prior to dosing | ceDNA—hFIX | | | | 4.585 mL/kg | pre-dose, 15 |
| 5 | 1 | DPH and Dex | LNP Formulation #3 | 0.05 | 0.01 | | | minutes, 6 |
| | | 30 minutes | ss-OP4-GalNac: | | | | | and 24-hours |
| | | prior to dosing | ceDNA—hFIX | | | | | post-dose |

Gr. = Group;
No. = Number;
An. = Animal;
Conc. = Concentration;
DPH = Diphenhydramine;
Dex = Dexamethasone;
LNP = Lipid nanoparticle

### Dosing Formulation

Dexamethasone and diphenhydramine were used at stock concentration. Formulations were be mixed (pipetting or stirred) prior to administration to distribute particulates of oral gavage suspension. The test articles were provided as follows: LNP Formulation #1 was provided as a 0.5 mg/mL sterile stock solution; LNP Formulation #2 was provided as a 1 mg/mL sterile stock solution; LNP Formulation #3 was provided as a 1 mg/mL sterile stock solution. On the day of dosing, the test article was removed from the refrigerator and was allowed to reach room temperature. Stock solutions were diluted before dosing to achieve the test concentrations.

### Animals

Eight male Macaca fascicularis cynomolgus monkeys (Chinese origin), ages 2 to 4 years, and weighing approximately 2.0 to 3.5 kg were used. The monkeys were all non-naïve. All animals were quarantined and acclimated according to Testing Facility IACUC Guidelines and SOP, and were assigned to study at the appropriate time after release from quarantine. Animals were group housed in pairs or singly housed for the duration of the study at a temperature of 64° F. to 84° F., humidity of 30% to 70% and a light cycle of 12 hours light and 12 hours dark (except during designated procedures).

Study animals were provided Monkey Diet 5038 (Lab Diet) daily. For psychological/environmental enrichment, animals were provided with items such as perches, foraging devices and/or hanging devices, except during study procedures/activities. Additional enrichment, such as music, was also be provided. Each animal was offered food supplements (such as certified treats, fresh fruit and/or Prima Foraging Crumbles®) except when fasting. Animals were anesthetized as described below for liver and spleen biopsy procedures. At the conclusion of the study, all animals were returned to the colony.

### Route of Administration and Dosage Level

The route of administration was selected based on anticipated exposure in humans. The dose level was selected based on a previous nonhuman primate study and corresponding dose levels in mice. The initial dose level of 0.01 mg/kg was 50-fold lower than administered previously. Based on results from Groups 1, 2 and 3, the test articles and dose levels were assigned in an escalation design up to a dose of 0.1 mg/kg, which is 5-fold lower than previously administered.

Pretreatment: All animals in all Groups were administered diphenhydramine (5 mg/kg IV or IM) and dexamethasone (1 mg/kg, IV or IM) 30 minutes (±3 minutes) prior to the start of dosing.

Test article infusion: The Test Article was administered by IV infusion to restrained animals over an approximate 70-minute period. Doses were administered through either the saphenous or cephalic vein with a temporary IV catheter. The catheter was flushed with 0.5 mL of saline at the end of dosing. Dose volumes were calculated based on the most recent body weight and rounded to the nearest 0.1 mL. The end time of IV dose infusion was used to determine target times for blood sample and biopsy collection time points. Injection site, dosing start and finish times were recorded in the raw data.

### In Life Observations and Measurements

Animal health checks were performed at least twice daily, in which all animals were checked for general health, behavior and appearance. Body weights were recorded prior to dosing on Day −1 or Day 0. Weights were rounded to the nearest 0.1 kg. Clinical observations were recorded on Day 0 prior to the start of dosing, at least once during dosing and once following the completion of dosing and prior to liver and spleen biopsies on Day 1. Additional observations were recorded as needed.

Sample collection: Blood samples were collected from an appropriate peripheral vein (not the vein used for dosing).

Whole blood for cytokine analysis: whole blood samples were collected from a peripheral vein via direct needle puncture into SST tubes and were processed for serum according to Testing Facility SOP. Serum samples were stored at −80° C. until shipment for analysis. Complement analysis: whole blood samples were collected from a peripheral vein via direct needle puncture into K₂EDTA tubes and were processed for plasma according to Testing Facility SOP. Plasma samples were stored at −80° C. until shipment for analysis.

Anti-PEG IgG/IgM analysis: whole blood samples were collected from a peripheral vein via direct needle puncture into SST tubes and were processed for serum according to Testing Facility SOP. Serum samples were stored at −80° C. until shipment for analysis.

Liver enzyme analysis: whole blood samples were collected from a peripheral vein via direct needle puncture into SST tubes and were processed for serum according to Testing Facility SOP. Serum samples were analyzed by the Testing Facility laboratory for ALT and AST using an IDEXX Catalyst analyzer.

Coagulation analysis: whole blood samples were collected from a peripheral vein via direct needle puncture into sodium citrate tubes and were processed for plasma according to Testing Facility SOP. Samples were stored at −80° C. until transferred for analysis of PTT, aPTT and fibrinogen.

Liver and Spleen biopsy

The liver and spleen biopsy were only be collected from the highest dose in the last phase of dosing.

Biopsy sample handling: The liver and spleen biopsies were kept whole, placed into labeled tube containing 10% neutral buffered and were refrigerated (˜4° C.). Tissue in 10% NBF was refrigerated (˜4° C.) until shipped in sealed container on ice packs for processing.

### Results

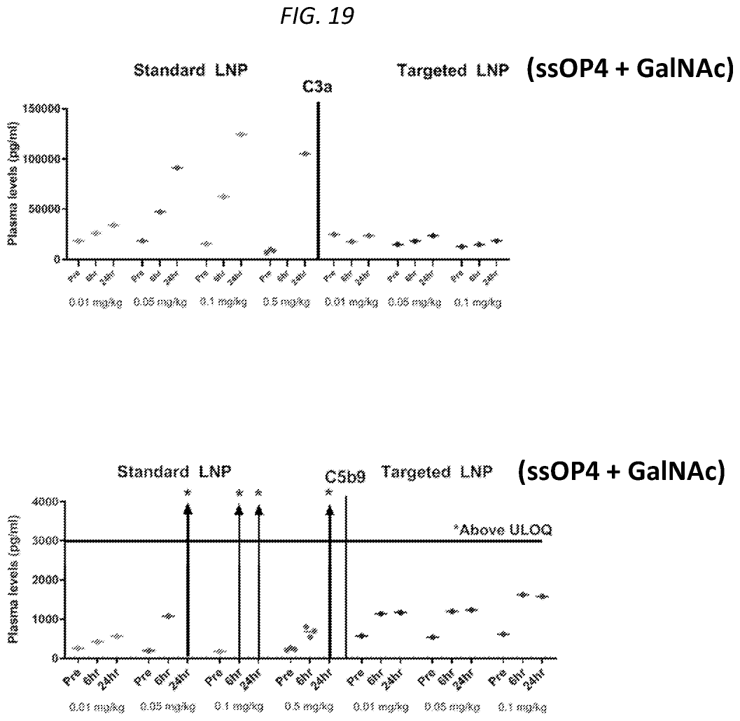

The effects of the ss-OP4 lipids (e.g., ss-OP, DOPC, cholesterol and PEG-DMG with an approximate molar ratio of 51:7:39:3, respectively) with GalNAc in the LNPs that contain ceDNA-hFactor IX (hFIX) on the complement pathway was compared with other standard non-cleavable lipids carrying similar ceDNA-hFIX.. Levels of C3a (pg/ml), one of the proteins formed by the cleavage of complement component 3, and levels of C5b9 (pg/ml), a complement activation end product were assessed in monkeys dosed with the standard non-cleavable LNPs (Formulations #1 and #2) and monkeys dosed with the targeted LNPs (Formulation #3) comprising ss-OP4 lipids, GalNAc and ceDNA-hFIX. Samples for analysis were taken pre-dose, at 6 hours and at 24 hours after dosing on day 0. As shown in FIG. 19, levels of C3a and C5b9 were significantly lower in animals treated with the ss-OP4-GalNacc LNPs compared to animals treated with the standard LNPs. A dramatic difference was observed at 24 hours post LNP dosing, where levels of C3a and C5b9 in animals treated with the standard LNPs were much higher than animals treated with the targeted LNPs. As shown in FIG. 19, the levels of C5b9 were above the upper limit of quantification after 24 hours in animals treated with the standard LNPs. This data demonstrates that the targeted LNPs comprising ss-OP, DOPC, cholesterol and PEG-DMG with an approximate molar ratio of 51:7:39:3, respectively, with GalNAc in the LNPs have an improved safety profile used in conjunction with ceDNA in terms of complement response.

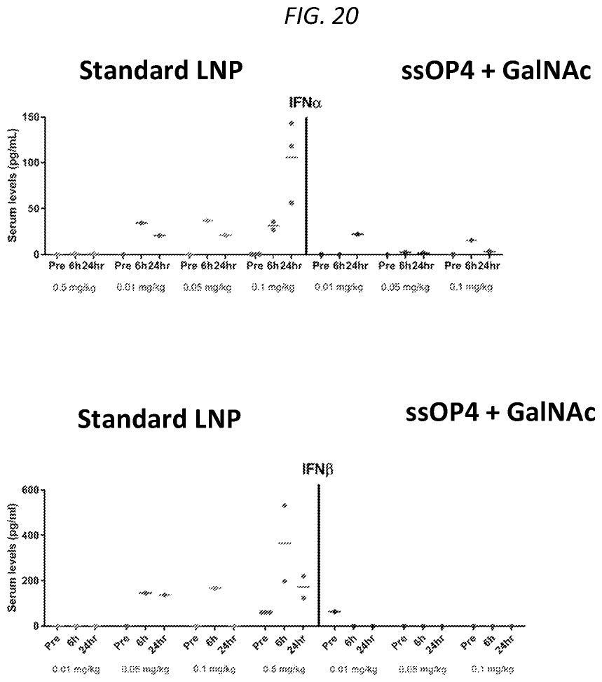

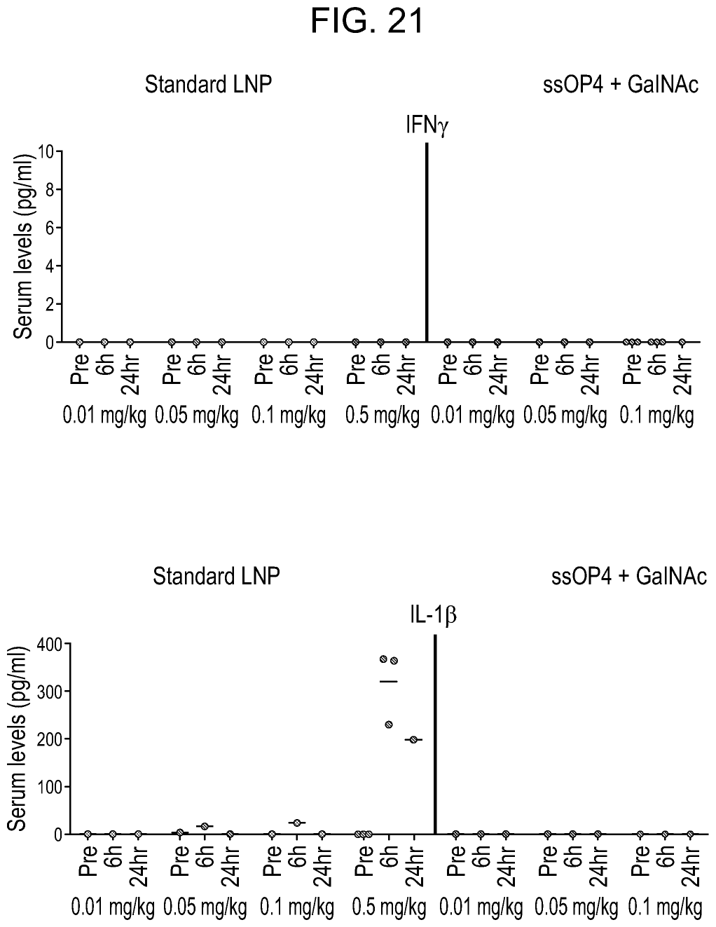

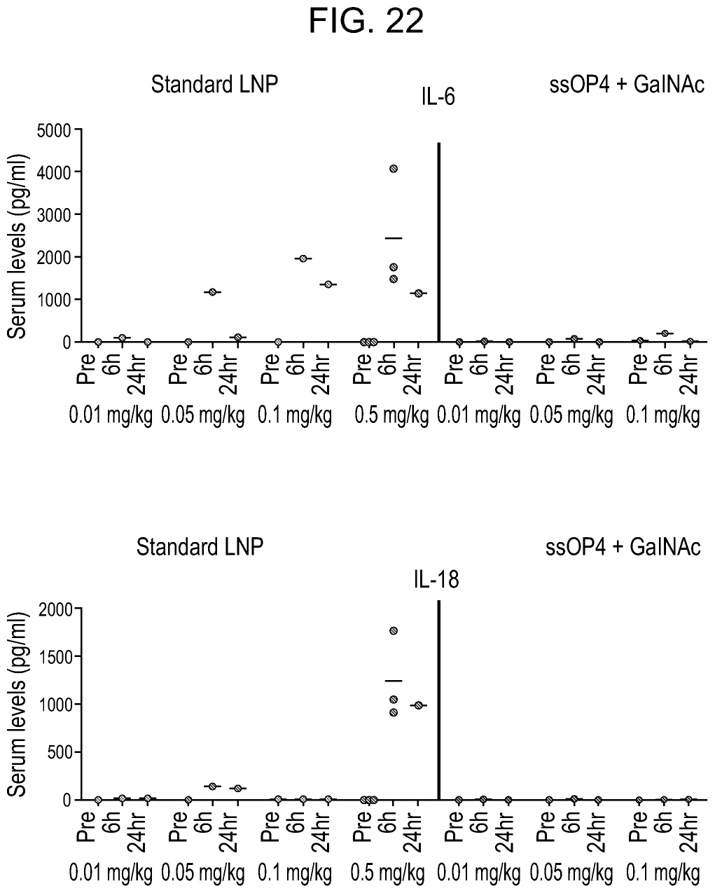

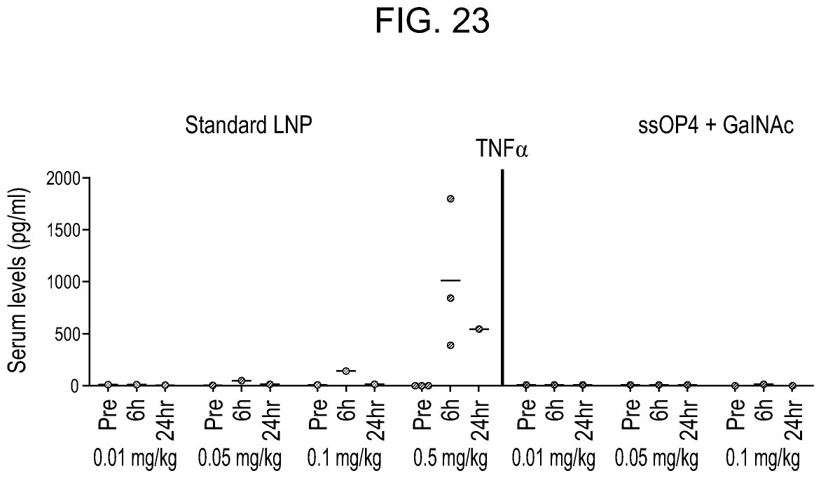

The effects of the ss-OP4 lipids used in conjunction with GalNAc in the LNPs on cytokine levels (pg/mL) in the serum of monkeys pre-dose, at 6 hours and at 24 hours after dosing on day 0 are shown in FIGS. 20-23. Levels of interferon alpha (IFNα) and interferon alpha (IFNα) (FIG. 20), interferon gamma (IFNγ) and interleukin-1 beta (IL-1β) (FIG. 21), IL-6 and IL-18 (FIG. 22) and tumor necrosis factor alpha (TNFα) (FIG. 23) were determined over a range of doses (0.01 mg/kg, 0.05 mg/kg, 0.1 mg/kg, 0.5 mg/kg). As shown in FIGS. 20-23, cytokine levels were significantly lower in the ss-OP4+GalNac:ceDNA-hFIX dose groups as compared to the standard LNP:ceDNA-hFIX dose group.

Taken together, the results demonstrate that ceDNA carrying an exogenous DNA (e.g., Factor IX) formulated in ss-OP4 with GalNAc showed a much improved safety profile in a non-human primate model in terms of complement and proinflammatory cytokine responses.

### Example 12: Evaluation of Safety and Transgene Expression of ceDNA LNP Formulations Injected Subretinally in a Rat Model

An in vivo study was performed to determine the safety and the amount of transgene expression in the retina following subretinal injection in both eyes using ceDNA lipid nanoparticle (LNP) formulations comprising ssOP4-formulated firefly luciferase (fLuc) mRNA or ssOP4-formulated ceDNA expressing luciferase (CpG minimized;) as the cationic lipid component.

Exemplary lipid nanoparticle formulations were prepared according to Example 6 and tested in vivo in a rat model. Male Sprague Dawley Rats were divided into 6 study groups, with 5 mice per group. All animals were assigned to study groups according to Powered Research Standard Operating Procedures (SOPs). All animals were pre-dosed with 0.5 mg/kg methylprednisolone, by intraperitoneal (IP) route of administration. Administration was by subretinal injection in both eyes (OD=right eye and OS=left eye).

The study design is outlined below in Table 17.

TABLE 17

| | | OS Dose | Volume | | OD Dose | Volume |
|---|---|---|---|---|---|---|
| Group | OS Tx | (ug or vg) | (ul) | OD Tx | (ug or vg) | (ul) |
| 1 | Non-treated | 0 | N/A | Vehicle | 0 | 2.5 |
| 2 | ss-OP4/Luc mRNA | 0.6 | 2.5 | ss-OP4/Luc mRNA | 0.6 | 2.5 |
| 3 | ss-OP4/Luc mRNA | 0.2 | 2.5 | ss-OP4/Luc mRNA | 0.2 | 2.5 |
| 4 | ss-OP4/ceDNA-luc | 0.6 | 2.5 | ss-OP4/ceDNA-luc | 0.6 | 2.5 |
| 5 | ss-OP4/Me ceDNA-luc | 0.6 | 2.5 | ss-OP4/Me ceDNA-luc | 0.6 | 2.5 |

The study details are set forth below.

Sprague Dawley rats (N=30 and 2 spare, male, ˜7-8 weeks of age and 150-200 g weight at first dosing) were obtained from Charles River Laboratories. Animals were observed for mortality and morbidity daily. Body weights for all animals were recorded at baseline (pre-dose) and at necropsy.

Treatment: Male Sprague Dawley rats received subretinal (subR) injections of 0.6 ug of ss-OP4-formulated firefly luciferase (fLuc) mRNA (N1-methyl-pseudouridine modified), ss-OP4-formulated ceDNA-luc (ADVM-Luc ceDNA; ceDNA encoding a CAG-fLuc expression cassette)—in both the right eye and the left eye. A non-treated group served as a control.

Surgical Procedure: On the day of the surgical procedure, rats were given buprenorphine 0.01-0.05 mg/kg sub-cutaneously (SQ). Animals were also given a cocktail of tropicamide (1.0%) and Phenylepherine (2.5%) topically to dilate and proptose the eyes. Animals were then tranquilized for the surgical procedure with a ketamine/xylazine cocktail, and one drop of 0.5% proparacaine HCL was applied to both eyes. Eyes were prepared for aseptic surgical procedures. Alternatively, rats were tranquilized with inhaled isoflurane. The cornea was kept moistened using topical eyewash, and body temperature was maintained using hot pads as needed. A 2-mm-long incision through the conjunctiva and Tenon's capsule was made to expose the sclera. A small pilot hole using the tip of a 30 gauge needle was made in the posterior sclera for subretinal injection using a 32-34 gauge needle and Hamilton syringe. Following the procedure, 1 drop of Ofloxacin ophthalmic solution followed by eye lube was applied topically to the ocular surface and animals were allowed to recover from surgery. If at any time during the surgical procedure, the surgeon determined the injection was suboptimal, or not successful, the animal was euthanized and replaced.

Ocular Examination: Ocular examination was performed using a slit lamp biomicroscope to evaluate ocular surface morphology at the timepoints indicated as follows in Table 18. All eyes designated for IHC were selected 24 h prior to sacrifice.

TABLE 18

| Day | n | Procedure |
|---|---|---|
| 3 | 1 | whole globe/group was collected and stained for |
| | | IHC/cryo (OS) or flash frozen for ddPCR analysis |
| | | of ceDNA (OD)** |
| 7 | 1 | whole globe/group was collected and stained for |
| | | IHC/cryo (OS) or flash frozen for ddPCR analysis |
| | | of ceDNA (OD)** |
| 28 | 2 or 4 | Remaining whole globes were collected for IHC/cryo |
| | | (n = 2/group) or flash frozen for ddPCR |
| | | (n = 4/group) |

Table 19 shown below indicates the scoring method that was used to assess anterior segment inflammation.

TABLE 19
Clinical Grading of Anterior Segment Inflammation in the Rat

| Grade^{a} | Criteria |
|---|---|
| 0 | No disease; eye is translucent and reflects light (red reflex) |
| 0.5 (trace) | Dilated blood vessels in the iris |
| 1 | Engorged blood vessels in the iris; abnormal pupil contraction |
| 2 | Hazy anterior chamber; decreased red reflux |
| 3 | Moderately opaque anterior chamber, but pupil still visible; |
| | dull red reflex |
| 4 | Opaque anterior chamber and obscured pupil; red reflex |
| | absent; proptosis |

^{a}Each higher grade includes the criteria of the preceding one.

Endpoints: The following endpoints were evaluated:
Body weights, mortality, clinical observations
Full Ocular Exams (OEs): Baseline, Day 8 and Day 21
Gross clinical observations: discharge, squinting, chemosis, scope analysis with anterior photos
Optical Coherence Tomography (OCT): Baseline (post-injection), Day 7, and Day 21
IVIS Imaging: Day 1, Day 3 and Day 14
Tissue (whole globes) collected for IHC (Iba1, Rho, DAPI) and ddPCR (Luc mRNA) as follows:

Day 3—N=1, OS immunohistochemistry (IHC), OD PCR

Day 7—N=1, OS IHC, OD PCR

Day 28—N=1, OU (both eyes) IHC; rest PCR

In-life Imaging: On days as indicated above, all animals underwent IVIS imaging procedures of the eye to quantify and determine luciferase expression. The substrate luciferin was injected intraperitoneally (0.15 mg/g), and the rats were imaged approximately 5-10 minutes after injection. Total flux (photons/sec), and average radiance (photons/sec/cm/sr) measurements from an elipsoid ROI around each eye were provided in a separate data report, along with all associated living image files. For all animals, each eye was imaged separately. Animals were imaged on their side.

Optical Coherence Tomography (OCT): On days as indicated above, all animals underwent OCT imaging procedures of the posterior section of the eye, to determine subretinal injection success and changes over time. Eyes were dilated using a cocktail of tropicamide HCL 1% and phenylephrine hydrochloride 2.5% for OCT 15 minutes prior to examination. Total retinal thickness and ONL thickness was measured at three positions (left, right, and center) from two OCT scans: one that goes through the injection site (bleb) and one that does not. All numerical thickness values were provided in a separate data report (spreadsheet), along with all associated/annotated OCT images.

Tissue Collections: One animal per group was euthanized on Days 3 and 7. The remaining animals were euthanized on Day 28 post-injection. Following euthanasia, the eyes were enucleated. Eyes were flash frozen in liquid nitrogen and were stored at −80° C. until dissection. The neurosensory retina was separated from the RPE/choroid/sclera. The neurosensory retina and RPE/choroid/sclera samples from each eye were collected into individual pre-weighed tubes and a tissue weight was obtained.

Histopathology: Eyes designated for cryosectioning were fixed for 4 hours at room temperature in 4% paraformaldehyde in separately labeled vials. Eyes were then transferred into 1× phosphate-buffered saline (PBS), and either embedded immediately in 3% agarose/5% sucrose and sunk overnight in 30% sucrose at 4 C or stored in 1×PBS until embedding the following day. Blocks were sectioned and processed for immunohistochemistry or hematoxylin and eosin staining. Slides designated for immunohistochemistry were stained with antibodies against Rhodopsin and Iba-1, alongside DAPI for nuclear localization. Remaining slides were stained with hematoxylin and eosin.

Results: Luciferase expression was determined by total flux (photons/second) using an IVIS Lumina S5 in vivo imaging system (Perkin Elmer), on days 1, 3 and 14. FIG. 24 shows that luciferase expression in the ss-OP4: Luc mRNA group was increased compared to vehicle control on days 1 and 3, demonstrating luciferase expression in the Luc mRNA group compared to control. By day 14, luciferase expression in the ss-OP4: Luc mRNA group decreased to levels similar to control. As shown in FIG. 24, luciferase expression in the ss-OP4: ceDNA-luc (a ceDNA encoding a CAG-fLuc expression cassette) group was increased compared to vehicle control on days 1, 3 and 14, demonstrating prolonged luciferase transgene expression in the ceDNA CAG-fLuc formulation group. FIG. 25 shows representative IVIS images. Notably, these results demonstrate that another nucleic acid (mRNA) can be delivered with the cleavable lipids described herein, in particular mRNA in an ss-OP4 formulation as described herein.

### Example 13: In Vitro Phagocytosis Assay for Functional Assessment of Formulations

An in vitro phagocytosis assay was performed using the ceDNA lipid nanoparticle (LNP) formulations comprising MC3, MC3-5% DSG-PEG2000 (1,2-Distearoyl-rac-glycero-3-methylpolyoxyethylene) (abbreviated as “5DSG”) and ss-OP4 as the cationic lipid component.

FIG. 14 shows a schematic of the phagocytosis assay for the ceDNA LNPs treated with 0.1% DiD (DiIC18(5); 1,1′-dioctadecyl-3,3,3′,3′-tetramethylindodicarbocyanine, 4-chlorobenzenesulfonate salt) lipophilic carbocyanine dye, where different concentrations of ceDNA (200 ng, 500 ng, 1 μg and 2 μg) were used in the MC3, MC3-5DSG or ss-OP4 LNPs, in the presence or absence of 10% human serum (+serum) and introduced to macrophage differentiated from THP-1 cells.

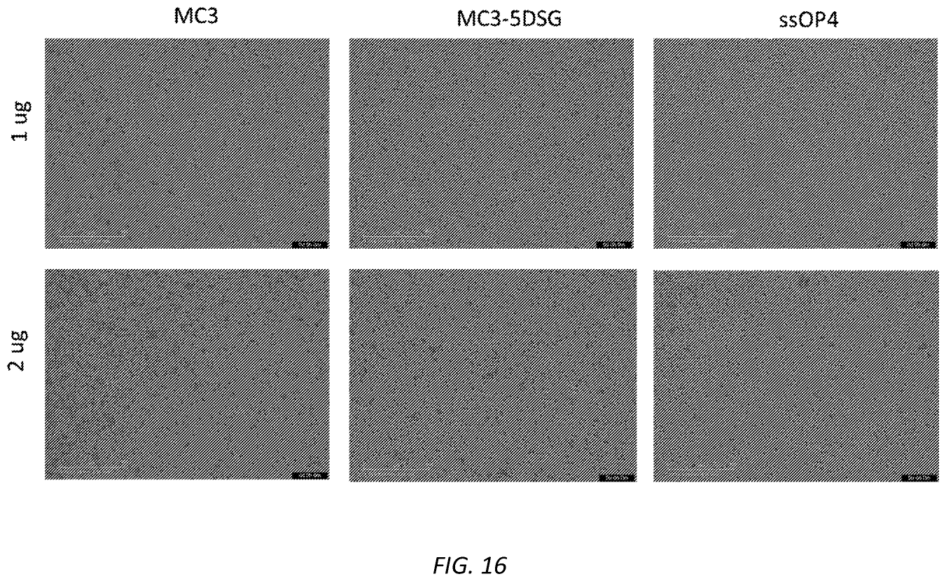

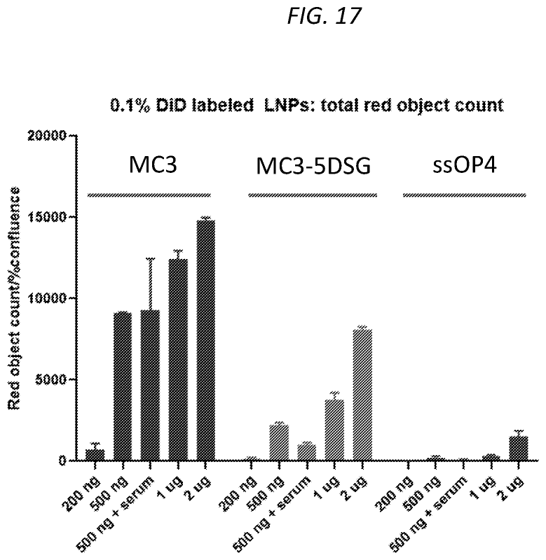

In FIG. 15 and FIG. 16, phagocytic cells that internalized ceDNA appear in red fluorescence. As shown in FIG. 15 and FIG. 16, the ss-OP4 LNPs comprising ceDNA were highly associated with the lowest number of fluorescent phagocytotic cells. Thus, without being bound by theory, it is thought that the ss-OP4 LNPs were better able to avoid phagocytosis by immune cells as compared to the MC3-5DSG and MC3 LNPs. FIG. 17 is a graph showing quantification of phagocytosis (by red object count/% confluence) for ss-OP4, MC3-5DSG and MC3 LNPs. It is noted that 0.1% DiD was used because in the 0.1% condition, phagocytotic cells exhibited intensity of red fluorescence in a dose dependent manner according to cell number.

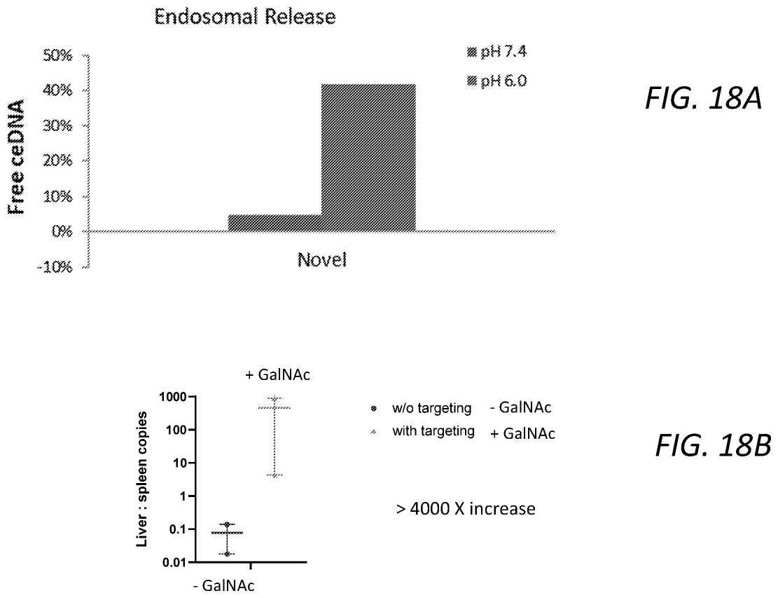

Indeed, a synergistic effect occurs between the ceDNA formulated in SS-cleavable lipid (e.g., ss-OP4) and GalNAc such that the ceDNA-LNPs comprising SS-cleavable lipid and GalNAc of the present invention exhibit approximately 4,000-fold greater hepatocyte targeting compared to ceDNA formulated in SS-cleavable lipid only (ss-OP4) (FIG. 18B), while ceDNA formulated in other cationic lipids with GalNAc demonstrated merely 10 to 100-fold greater hepatocyte targeting (data not shown). Both ss-OP4 and other cationic lipid LNPs showed a similar level of endosomal escape (FIG. 18A). These data suggest that SS-cleavable lipid formulated in ceDNA not only improves expression and exert positive effects on mitigating proinflammatory immune responses, but also demonstrates a synergistic effect in targeting ceDNA LNPs to a specific organ such as liver with a tissue specific ligand (e.g., liver specific ligand, GalNAc).

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

What is claimed is:

1. A pharmaceutical composition comprising lipid nanoparticle (LNP), wherein the LNP comprises a SS-cleavable lipid and a closed-ended DNA (ceDNA).

2. The pharmaceutical composition of claim 1, wherein the SS-cleavable lipid comprises a disulfide bond and a tertiary amine.

3. The pharmaceutical composition of any one of the previous claims, wherein the SS-cleavable lipid comprises an ss-OP lipid of Formula I:

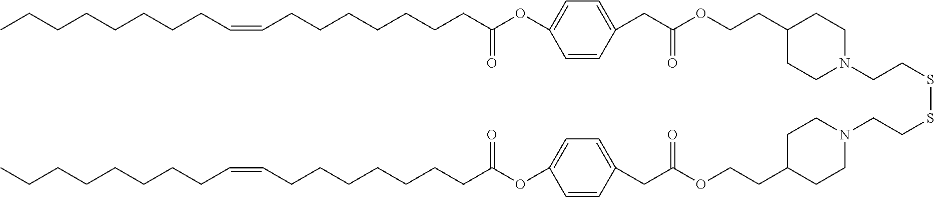 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

4. The pharmaceutical composition of any one of the previous claims, wherein the LNP further comprises a sterol.

5. The pharmaceutical composition of claim 4, wherein the sterol is a cholesterol.

6. The pharmaceutical composition of any one of the previous claims, wherein the LNP further comprises a polyethylene glycol (PEG).

7. The pharmaceutical composition of claim 6, wherein the PEG is 1-(monomethoxy-polyethyleneglycol)-2,3-dimyristoylglycerol (PEG-DMG).

8. The pharmaceutical composition of any one of the previous claims, wherein the LNP further comprises a non-cationic lipid.

9. The pharmaceutical composition of claim 8, wherein the non-cationic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof.

10. The pharmaceutical composition of claim 9, wherein the non-cationic lipid is selected from the group consisting of dioleoylphosphatidylcholine (DOPC), distearoylphosphatidylcholine (DSPC), and dioleoyl-phosphatidylethanolamine (DOPE).

11. The pharmaceutical composition of claim 10, wherein the PEG or PEG-lipid conjugate is present at about 1.5% to about 3%.

12. The pharmaceutical composition of any one of claims 10 and 11, wherein the cholesterol is present at a molar percentage of about 20% to about 40%, and wherein the SS-cleavable lipid is present at a molar percentage of about 80% to about 60%.

13. The pharmaceutical composition of claim 12, wherein the cholesterol is present at a molar percentage of about 40%, and wherein the SS-cleavable lipid is present at a molar percentage of about 50%.

14. The pharmaceutical composition of any one of claims 1-3, wherein the composition further comprises a cholesterol, a PEG or PEG-lipid conjugate, and a non-cationic lipid.

15. The pharmaceutical composition of claim 14, wherein the PEG or PEG-lipid conjugate is present at about 1.5% to about 3%.

16. The pharmaceutical composition of claim 14 or claim 15, wherein the cholesterol is present at a molar percentage of about 30% to about 50%.

17. The pharmaceutical composition of any one of claims 14-16, wherein the SS-cleavable lipid is present at a molar percentage of about 42.5% to about 62.5%.

18. The pharmaceutical composition of any one of claims 14-17, wherein the non-cationic lipid is present at a molar percentage of about 2.5% to about 12.5%.

19. The pharmaceutical composition of any one of claims 14-18, wherein the cholesterol is present at a molar percentage of about 40%, the SS-cleavable lipid is present at a molar percentage of about 52.5%, the non-cationic lipid is present at a molar percentage of about 7.5%, and wherein the PEG is present at about 3%.

20. The pharmaceutical composition of any of the previous claims, wherein the composition further comprises dexamethasone palmitate.

21. The pharmaceutical composition of any one of the previous claims, wherein the LNP is in size ranging from about 50 nm to about 110 nm in diameter.

22. The pharmaceutical composition of any one of claims 1-20, wherein the LNP is less than about 100 nm in size.

23. The pharmaceutical composition of claim 22, wherein the LNP is less than about 70 nm in size.

24. The pharmaceutical composition of claim 23, wherein the LNP is less than about 60 nm in size.

25. The pharmaceutical composition of any one of the previous claims, wherein the composition has a total lipid to ceDNA ratio of about 15:1.

26. The pharmaceutical composition of any one of the previous claims, wherein the composition has a total lipid to ceDNA ratio of about 30:1.

27. The pharmaceutical composition of any one of the previous claims, wherein the composition has a total lipid to ceDNA ratio of about 40:1.

28. The pharmaceutical composition of any one of the previous claims, wherein the composition has a total lipid to ceDNA ratio of about 50:1.

29. The pharmaceutical composition of any one of the previous claims, wherein the composition further comprises N-Acetylgalactosamine (GalNAc).

30. The pharmaceutical composition of claim 29, wherein the GalNAc is present in the LNP at a molar percentage of 0.5% of the total lipid.

31. The pharmaceutical composition of any one of the previous claims, wherein the composition further comprises about 10 mM to about 30 mM malic acid.

32. The pharmaceutical composition of claim 31, wherein the composition comprises about 20 mM malic acid.

33. The pharmaceutical composition of any one of the previous claims, wherein the composition further comprises about 30 mM to about 50 mM NaCl.

34. The pharmaceutical composition of claim 33, wherein the composition comprises about 40 mM NaCl.

35. The pharmaceutical composition of any one of claims 1-33, wherein the composition further comprises about 20 mM to about 100 mM MgCl₂.

36. The pharmaceutical composition of any one of the previous claims, wherein the ceDNA is closed-ended linear duplex DNA.

37. The pharmaceutical composition of any one of the previous claims, wherein the ceDNA comprises an expression cassette comprising a promoter sequence and a transgene.

38. The pharmaceutical composition of claim 37, wherein the ceDNA comprises expression cassette comprising a polyadenylation sequence.

39. The pharmaceutical composition of any one of claims 36-38, wherein the ceDNA comprises at least one inverted terminal repeat (ITR) flanking either 5′ or 3′ end of said expression cassette.

40. The pharmaceutical composition of claim 39, wherein said expression cassette is flanked by two ITRs, wherein the two ITRs comprise one 5′ ITR and one 3′ ITR.

41. The pharmaceutical composition of claim 39, wherein the expression cassette is connected to an ITR at 3′ end (3′ ITR).

42. The pharmaceutical composition of claim 39, wherein the expression cassette is connected to an ITR at 5′ end (5′ ITR).

43. The pharmaceutical composition of claim 39, wherein at least one of 5′ ITR and 3′ ITR is a wild-type AAV ITR.

44. The pharmaceutical composition of claim 39, wherein at least one of 5′ ITR and 3′ ITR is a modified ITR.

45. The pharmaceutical composition of claim 39, wherein the ceDNA further comprises a spacer sequence between a 5′ ITR and the expression cassette.

46. The pharmaceutical composition of claim 39, wherein the ceDNA further comprises a spacer sequence between a 3′ ITR and the expression cassette.

47. The pharmaceutical composition of claim 45 or claim 46, wherein the spacer sequence is at least 5 base pairs long in length.

48. The pharmaceutical composition of claim 47, wherein the spacer sequence is 5 to 100 base pairs long in length.

49. The pharmaceutical composition of claim 47, wherein the spacer sequence is 5 to 500 base pairs long in length.

50. The pharmaceutical composition of any one of the previous claims, wherein the ceDNA has a nick or a gap.

51. The pharmaceutical composition of claim 39, wherein the ITR is an ITR derived from an AAV serotype, derived from an ITR of goose virus, derived from a B19 virus ITR, a wild-type ITR from a parvovirus.

52. The pharmaceutical composition of claim 51, wherein said AAV serotype is selected from the group comprising of AAV1, AAV2, AAV3, AAV4, AAV5, AAV6, AAV7, AAV8, AAV9, AAV10, AAV11 and AAV12.

53. The pharmaceutical composition of claim 39, wherein the ITR is a mutant ITR, and the ceDNA optionally comprises an additional ITR which differs from the first ITR.

54. The pharmaceutical composition of claim 39, wherein the ceDNA comprises two mutant ITRs in both 5′ and 3′ ends of the expression cassette, optionally wherein the two mutant ITRs are symmetric mutants.

55. The pharmaceutical composition of any one of the previous claims, wherein the ceDNA is a CELiD, DNA-based minicircle, a MIDGE, a ministering DNA, a dumbbell shaped linear duplex closed-ended DNA comprising two hairpin structures of ITRs in the 5′ and 3′ ends of an expression cassette, or a Doggybone™ DNA.

56. The pharmaceutical composition of any one of the previous claims, further comprising a pharmaceutically acceptable excipient.

57. A method of treating a genetic disorder in a subject, the method comprising administering to the subject an effective amount of the pharmaceutical composition according to any of the previous claims.

58. The method of claim 50, wherein the subject is a human.

59. The method of claim 57 or claim 58, wherein the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson's disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, and Cathepsin A deficiency.

60. The method of claim 59, wherein the genetic disorder is Leber congenital amaurosis (LCA).

61. The method of claim 60, wherein the LCA is LCA10.

62. The method of claim 59, wherein the genetic disorder is Niemann-Pick disease.

63. The method of claim 59, wherein the genetic disorder is Stargardt macular dystrophy.

64. The method of claim 59, wherein the genetic disorder is glucose-6-phosphatase (G6Pase) deficiency (glycogen storage disease type I) or Pompe disease (glycogen storage disease type II).

65. The method of claim 59, wherein the genetic disorder is hemophilia A (Factor VIII deficiency).

66. The method of claim 59, wherein the genetic disorder is hemophilia B (Factor IX deficiency).

67. The method of claim 59, wherein the genetic disorder is hunter syndrome (Mucopolysaccharidosis II).

68. The method of claim 59, wherein the genetic disorder is cystic fibrosis.

69. The method of claim 59, wherein the genetic disorder is dystrophic epidermolysis bullosa (DEB).

70. The method of claim 59, wherein the genetic disorder is phenylketonuria (PKU).

71. The method of claim 59, wherein the genetic disorder is hyaluronidase deficiency.

72. The method of any one of claims 57-71, further comprising administering an immunosuppressant.

73. The method of claim 72, wherein the immunosuppressant is dexamethasone.

74. The method of any one of claims 57-73, wherein the subject exhibits a diminished immune response level against the pharmaceutical composition, as compared to an immune response level observed with an LNP comprising MC3 as a main cationic lipid, wherein the immune response level against the pharmaceutical composition is at least 50% lower than the level observed with the LNP comprising MC3.

75. The method of claim 74, wherein the immune response is measured by detecting the levels of a pro-inflammatory cytokine or chemokine.

76. The method of claim 75, wherein the pro-inflammatory cytokine or chemokine is selected from the group consisting of IL-6, IFNα, IFNγ, IL-18, TNFα, IP-10, MCP-1, MIP1α, MIP1β, and RANTES.

77. The method of claim 76, wherein at least one of the pro-inflammatory cytokines is under a detectable level in serum of the subject at 6 hours after the administration of the pharmaceutical composition.

78. The method of any one of claims 57-77, wherein the LNP comprising the SS-cleavable lipid and the closed-ended DNA (ceDNA) is not phagocytosed; or exhibits diminished phagocytic levels by at least 50% as compared to phagocytic levels of LNPs comprising MC3 as a main cationic lipid administered at a similar condition.

79. The method of claim 78, wherein the SS-cleavable lipid is ss-OP of Formula I.

80. The method of claim 79, wherein the LNP further comprises cholesterol and a PEG-lipid conjugate.

81. The method of claim 80, wherein the LNP further comprises a noncationic lipid.

82. The method of claim 81, wherein the noncationic lipid is selected from the group consisting of dioleoylphosphatidylcholine (DOPC), distearoylphosphatidylcholine (DSPC), and dioleoyl-phosphatidylethanolamine (DOPE).

83. The method of any of claim 80 or claim 81, wherein the LNP further comprises N-Acetylgalactosamine (GalNAc).

84. The method of claim 83, wherein the GalNAc is present in the LNP at a molar percentage of 0.5% of the total lipid.

85. A method of increasing therapeutic nucleic acid targeting to the liver of a subject in need of treatment, the method comprising administering to the subject an effective amount of a lipid nanoparticle LNP comprising therapeutic nucleic acid, ss-cleavable lipid, sterol, and polyethylene glycol (PEG) and N-Acetylgalactosamine (GalNAc).

86. The method of claim 85, wherein the PEG is 1-(monomethoxy-polyethyleneglycol)-2,3-dimyristoylglycerol (PEG-DMG).

87. The method of claim 85, wherein the LNP further comprises a non-cationic lipid.

88. The method of claim 87, wherein the non-cationic lipid is selected from the group consisting of dioleoylphosphatidylcholine (DOPC), distearoylphosphatidylcholine (DSPC), and dioleoyl-phosphatidylethanolamine (DOPE).

89. The method of claim 85, wherein the GalNAc is present in the LNP at a molar percentage of 0.5% of the total lipid.

90. The method of claim 85, wherein the subject is suffering from a genetic disorder.

91. The method of claim 90, wherein the genetic disorder is hemophilia A (Factor VIII deficiency).

92. The method of claim 90, wherein the genetic disorder is hemophilia B (Factor IX deficiency).

93. The method of claim 90, wherein the genetic disorder is phenylketonuria (PKU).

94. The method of claim 85, wherein the therapeutic nucleic acid is selected from the group consisting of minigenes, plasmids, minicircles, small interfering RNA (siRNA), microRNA (miRNA), antisense oligonucleotides (ASO), ribozymes, ceDNA, ministring, Doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof.

95. The method of claim 85, wherein the therapeutic nucleic acid is ceDNA.

96. The method of claim 95, wherein the ceDNA comprises an expression cassette comprising a promoter sequence and a transgene.

97. The method of claim 96, wherein the ceDNA comprises at least one inverted terminal repeat (ITR) flanking either 5′ or 3′ end of said expression cassette.

98. The method of claim 95, wherein the ceDNA is selected from the group consisting of a CELiD, a MIDGE, a ministering DNA, a dumbbell shaped linear duplex closed-ended DNA comprising two hairpin structures of ITRs in the 5′ and 3′ ends of an expression cassette, or a Doggybone™ DNA, wherein the ceDNA is capsid free and linear duplex DNA.

99. A method of mitigating a complement response in a subject in need of treatment with a therapeutic nucleic acid (TNA), the method comprising administering to the subject an effective amount of a lipid nanoparticle (LNP) comprising the TNA, a ss-cleavable lipid, a sterol, polyethylene glycol (PEG), and N-Acetylgalactosamine (GalNAc).

100. The method of claim 99, wherein the subject is suffering from a genetic disorder.

101. The method of claim 100, wherein the genetic disorder is selected from the group consisting of sickle-cell anemia, melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson's disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, and Cathepsin A deficiency.

102. The method of claim 99, wherein the therapeutic nucleic acid is selected from the group consisting of minigenes, plasmids, minicircles, small interfering RNA (siRNA), microRNA (miRNA), antisense oligonucleotides (ASO), ribozymes, ceDNA, ministring, Doggybone™ protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, DNA viral vectors, viral RNA vector, non-viral vector and any combination thereof.

103. The method of claim 102, wherein the ceDNA is selected from the group consisting of a CELiD, a MIDGE, a ministering DNA, a dumbbell shaped linear duplex closed-ended DNA comprising two hairpin structures of ITRs in the 5′ and 3′ ends of an expression cassette, or a Doggybone™ DNA, wherein the ceDNA is capsid free and linear duplex DNA.

104. The method of claim 99, wherein the PEG is 1-(monomethoxy-polyethyleneglycol)-2,3-dimyristoylglycerol (PEG-DMG).

105. The method of claim 104, wherein the PEG is present in the LNP at a molecular percentage of about 2 to 4%.

106. The method of claim 105, wherein the PEG is present in the LNP at a molecular percentage of about 3%.

107. The method of claim 99, wherein the LNP further comprises a non-cationic lipid.

108. The method of claim 107, wherein the non-cationic lipid is selected from the group consisting of dioleoylphosphatidylcholine (DOPC), distearoylphosphatidylcholine (DSPC), and dioleoyl-phosphatidylethanolamine (DOPE).

109. The method of claim 99, wherein the GalNAc is present in the LNP at a molar percentage of about 0.3 to 1% of the total lipid.

110. The method of claim 107, wherein the GalNAc is present in the LNP at a molar percentage of about 0.5% of the total lipid.
